(19) United States
(12) Patent Application Publication
(10) Pub. No.: US 2024/0159768 A1
(43) Pub. Date: May 16, 2024

(54) NANOPORE PROTEOMICS

(71) Applicant: Rijksuniversiteit Groningen, Groningen (NL)

(72) Inventors: Florian Leonardus Rudolfus LUCAS, Leuven (BE); Roderick Corstiaan Abraham VERSLOOT, Groningen (NL); Giovanni MAGLIA, Groningen (NL)

(21) Appl. No.: 18/512,856

(22) Filed: Nov. 17, 2023

Related U.S. Application Data
(63) Continuation of application No. PCT/NL2022/050266, filed on May 18, 2022.

(30) Foreign Application Priority Data
May 18, 2021 (EP) 21174437.0

(51) Int. Cl. G01N 33/68; G01N 33/543

(52) U.S. Cl. CPC G01N 33/6818; G01N 33/54373

(57) ABSTRACT

The invention relates to the field of genetically engineered nanopores and the use thereof in analyzing biopolymers and other (biological) compounds. Provided is a proteinaceous nanopore comprising a mutant pore-forming toxin, or a pore-forming fragment thereof, wherein the lumen-facing recognition region of the pore-forming protein or fragment thereof comprises one or more substitution(s) of lumen-facing amino acid(s) in the recognition region corresponding to amino acids 10-20 of Fragaceatoxin C (FraC), to a natural or non-natural aromatic amino acid residue.

### CROSS-REFERENCE

This application is a continuation of International Patent Application No. PCT/NL2022/050266, filed May 18, 2022, which claims the benefit of European Application No. 21174437.0, filed May 18, 2021, each of which is entirely incorporated herein by reference.

### SEQUENCE LISTING

The instant application contains a Sequence Listing which has been submitted electronically in XML format and is hereby incorporated by reference in its entirety. Said XML copy, created on Jan. 22, 2024, is named 64828-705_301_SL.xml and is 113,571 bytes in size.

### BACKGROUND

The invention relates generally to the field of nanopores and the use thereof in analyzing biopolymers and other (biological) compounds. In particular, it relates to genetically engineered nanopores, and their improved performance in peptide capture and recognition.

### DETAILED DESCRIPTION

Nanopores have become potential candidates for inexpensive, high-throughput and/or portable protein detectors. In recent years, they have shown to work analogous to mass-analysers, not only for model analytes, such as polyethylene glycol (PEG), but also for biological polymers such as peptides and proteins (Robertson et al. Proc Natl Acad Sci USA. 2007). Biological nanopores have been shown to be particularly suitable for the detection and discrimination of small molecules based on the signal they produce when an analyte translocates through the recognition site of the nanopore. This method, known as nanopore spectrometry (Chavis et al., ACS Sens, 2017), is dependent on the interaction between the pore surface and the analyte.

Pore forming proteins (PFPs) can be roughly classified into two major groups, α-PFPs or β-PFPs, which form pores by bundles of α-helices or by transmembrane β-barrels, respectively. Although members of either group of PFPs share a common general mode of pore formation, several evolutionarily unrelated families can be distinguished according to the structures of their soluble monomers.

The α-helical pore forming toxin family produced by sea anemones (actinoporins) are pore forming proteins with a mass of approximately 20 kDa (Anderluh et al. Toxicon, 2002). The sequence identity of the actinoporin family is high (60-80%) (Garcia-Ortega et al. Biochim Biophys Acta, 2011), and the mechanism of pore formation is thought to be largely similar, where pore formation is often dependent on the presence of sphingomyelin in the lipid bilayer. The activity of actinoporins can be traced to their α-helical transmembrane region, formed by the first 30-32 amino acids (FIG. 1A) (Ros et al. Biochimie, 2015). This region also contains the narrowest point-constriction site—of the pores (Huang et al. Nat Commun, 2019). The physiological properties of the actinoporin fragaceatoxin C (FraC)—such as the electro-osmotic flow (EOF) and the recognition volume (Huang et al. Nat Commun, 2017: Huang et al. Nat Commun, 2019)—can be engineered, making FraC a prime target to be developed for single molecule nanopore spectrometry. The interaction of the pore with biological analytes, however, is poorly understood.

Three main families of β-PFPs are the α-hemolysin family found predominately in Staphylococcus aureus, the MACPF/CDC protein superfamily, and PFPs exhibiting similarity to aerolysin, a well-studied toxin from the pathogenic bacterium A. hydrophila.

The detection of analytes and the sequencing of DNA using biological nanopores has seen major advances over recent years. The detection and sequencing of proteins with nanopores, however, is complicated by the complex physico-chemical structure of polypeptides, and the lack of understanding of the mechanism of capture and recognition of polypeptides by nanopores

The crystal structure of wild-type FraC (WtFraC) displays an oligomeric pore formed from 8 identical subunits (octameric) (Tanaka et al., Nat Commun, 2015). In previous work, we have shown that FraC is capable of forming different oligomeric forms-most notably the octameric (T1) and heptameric (T2)—with a distinct pore volume and range of detectable peptides (WO2020/055246; Huang et al. Nat Commun, 2019). In the same work we also demonstrated that the current observed from peptide translocation through WtFraC correlates with the mass of the peptide at a pH of 3.8, in 1 M KCl. However, the peptide blockades were fast—in the order of several micro seconds (average dwell time for Angiotensin 1 is 0.15±0.04 ms) (Huang et al. Nat Commun, 2019), which causes the majority of translocation events to remain undetected and detected events to be inaccurately characterized.

Therefore, a goal of the present invention is to improve the accuracy of characterizing individually captured peptides by a nanopore sensor. To that end, we engineered proteinaceous nanopores to improve the capture of unlabeled peptides, to increase the residence (dwell time) of peptides in the nanopore sensor, and to improve the discrimination between peptide species. In this invention we have also engineered proteins nanopores to improve peptide sensing under low pH conditions that are optimized for peptide detection.

It was surprisingly found that introducing one or more “bulky”/aromatic amino acids at precise positions within the lumen of nanopores can increase both the capture frequency of peptides and also largely improves the discrimination among peptides. For example, fragaceatoxin C (FraC) nanopores comprising subunits wherein a tyrosine, phenylalanine or tryptophan residue was introduced in the lumen-facing region was found to show an increased dwell time of peptides in the pore. Moreover, these ‘large aromatically modified” nanopores could detect and measure the peptides in a tryptic digest of lysozyme. Furthermore, the unique individual spectra of individual proteins could be assigned using advanced analytical methods such as spectral matching. Furthermore, we have adapted our combination of mutation engineering discoveries and detection conditions to other nanopore families, e.g. the beta-barrel pores, with quite different structures but similar size recognition regions in the lumen of the nanopores, and have found the improvement in peptide characterisation is universal.

These findings provide a proof of concept that the modified nanopores can be used as single molecule detector capable of label-free protein detection and fingerprinting. It provides the basis to improve the recognition and augment the capture of peptides by nanopores, which is important for developing a real-time and single-molecule volume-analyzer for peptide recognition and identification.

Accordingly, the invention relates to a proteinaceous nanopore comprising a mutant (or “modified”) transmembrane pore-forming toxin, e.g. of the actinoporin family, or a pore-forming fragment thereof, wherein the lumen-facing recognition region of the pore-forming protein or fragment thereof comprises one or more mutations to a natural or non-natural aromatic amino acid residue. A pore-forming toxin is typically an oligomer. The pore is preferably made up of several repeating subunits, such as 6, 7 or 8 subunits. The pore comprises a central channel when inserted into a membrane through which the ions may flow, for example when a potential is applied across the membrane. The subunits of the pore typically surround a central axis and contribute strands to a transmembrane α-helix bundle or channel. Thus, a modified proteinaceous nanopore in accordance with the invention comprises an oligomer (or “assembly”) of mutant pore-forming alpha-helical pore-forming subunits of the actinoporin family, or an oligomer of pore-forming fragment thereof.

The “lumen-facing recognition region”, herein also referred to as the “recognition area” or “water-facing region” of the pore, is meant to indicate the part of the nanopore that is involved in the sensing of an analyte that traverses the pore. The recognition region is typically a part of the central water-filled channel (the lumen) that is formed through the nanopore from cis to trans when inserted into a membrane such as a lipid bilayer. The recognition region can typically be identified structurally by the dimensions of the central channel. Suitable structures or structural models can be obtained or constructed by means known in the art, including from experimental x-ray diffraction structures, electron-microscopy structures, and computer modelling. The recognition region will be the region of the channel through the nanopore where the electric field lines concentrate and the presence of the analyte disrupts the most the ionic current flowing through the nanopore under an applied potential. For small peptide detection, the recognition region will preferably comprise the section/s of the nanopore channel with an internal diameter of less than 2 nanometers, and preferably less than 1 nanometer, so as to yield a significant deflection of the ionic current and sufficient residence/dwell time during analyte interaction. Many nanopores might have one or more narrow sections of small internal diameters (constriction/s) within the longer recognition region. Maximum sensitivity/ionic current deflection to analytes is typically achieved when the analyte interacts with the nanopore at or near a constriction. Therefore, maximal control of analyte detection can often be achieved by protein mutagenesis/engineering at or adjacent to the residues that comprise the constriction.

Alternatively, for proteins that are known to be nanopores (e.g. by homology searches or by experimental determination) but do not have suitable structural models, the recognition region can often be determined by computer modelling and/or homology mapping to the recognition region of other known nanopores using means known in the art. For example, the recognition region often includes or entirely resides within the transmembrane section of a membrane-protein nanopore (e.g. transmembrane sections comprised of beta-barrels or alpha-helical oligomers). Transmembrane beta-barrels and alpha-helices can be identified by means such as homology comparison to other known pores and by features such as amphipathic hydropathy maps for example.

Nanopore recognition regions can also be determined and/or confirmed experimentally by mutagenesis using well known means in the art. For example, the ionic current characteristics of different nanopores with different targeted mutations in the candidate recognition regions can be compared in electrophysiology experiments of the nanopores inserted into membranes. By varying the position of the mutations, and optionally measuring differences in response to control analytes, the recognition region can be mapped and characterized.

A pore of the invention is among others characterized in that the lumen-facing recognition region of the pore is engineered (by one or more natural or non-natural amino acid substitutions) to manipulate the internal dimensions/hydrophobicity/aromaticity of the pore, therewith increasing the dwell time and resolution for peptides traversing the nanopore.

Accordingly, the invention also provides a method of decreasing the translocation speed of a peptide analyte through a transmembrane (alpha-helical or beta-barrel) protein pore, comprising:
(a) increasing the net aromaticity of the lumen of the pore by substituting one or more non-aromatic amino acids with one or more aromatic amino acids, preferably wherein said substituting results in a proteinaceous nanopore as herein disclosed; and (b) passing the polypeptide through the pore, wherein increasing the net aromaticity decreases the translocation speed of the polypeptide through the pore.

As will be understood by a person skilled in the art, the one or more mutations of the invention can be introduced in a number of configurations to the nanopores or functional pore forming fragments thereof so as to produce the desired change/s in the recognition region of the assembled nanopore. For example, for oligomeric nanopores comprised of a number (e.g. 4, 5, 6, 7, 8, or more) of monomeric units, one or more mutations are made to all monomers used to assemble the nanopore, so that the assembled nanopore contains a ring of multiple identical mutations in the recognition region that is co-planar with the membrane and orthogonal to the direction of analyte passage. Alternatively, mutated monomers might be mixed with monomers containing no mutations or different mutations during nanopore oligomerisation to create “hetero-oligomeric” assembled nanopores with a controlled number of mutations.

Hence, the assembled pore may comprise 1, 2, 3, 4, 5, 6, 7, 8, 9 or more mutant monomers of the invention, depending on the number of oligomer units. Controlling the number of mutated units can be useful to reduce or otherwise modulate the extent/magnitude of the change to the recognition region. Means to selectively purify the population of hetero-oligomeric nanopores with the desired number of mutations from a mixture are known in the art (e.g. Gouaux, et al Proc Nal Acad Sci U S A 1994). For oligomeric nanopores that contain more than one type of monomeric unit that in the assembled form both contribute to the recognition region (e.g. bi-component Leukocidins, Spaan et al Nat Rev Microbiol 2017) the mutations might be made to one or both of the monomer species. For pores where monomers are fused together (e.g. either genetically or by other chemical conjugation means), for example in the case of dimers (Hammerstein et al, J Biol Chem 2011), then a person of skill in the art would understand that the desired number of mutations can be introduced to the final assembly by choosing how many of the monomer units to modify.

For monomeric protein nanopores (e.g. outer membrane porins) where a single protein strand makes up the transmembrane channel, it is understood that mutations can be made along the sequence at specific interspaced distances such that the assembled nanopore channel contains the required number of water-facing mutations, preferably on multiple beta-strands of the transmembrane section of the pore, most preferably on all beta-strand units to create a ring-like formation of mutations similar to that formed in a homo-oligomeric nanopore. For beta-barrel based nanopores it is understood that mutations might be introduced to either the down strand or the up strand or to both. For example, aromatic or acidic substitutions of the invention might be added to both the up and down strands of the beta-barrel so that they are approximately co-planar (same vertical position in the nanopore) to create a stronger effect in the recognition region. In other embodiments, multiple mutations of the invention can made (vertically relative to the membrane) along the alpha-helix or beta-strand of a monomer of an oligomeric pore or fragment thereof, for example to create stronger alterations to the recognition region.

Natural and non-natural aromatic amino acid residues are known in the art. In one embodiment, the non-natural aromatic amino acid is selected from the group consisting of 3,4-dihydroxy-L-phenylalanine, 3-iodo-L-tyrosine, triiodothyronine, L-thyroxine, phenylglycine (Phg) or nor-tyrosine (norTyr). Phg and norTyr. Suitable non-natural amino acids can include D-amino acids, Homo-amino acids (methylene), Beta-homo-amino acids, N-methyl amino acids, Alpha-methyl amino acids. A wide range of well-known non-natural amino acids are known in the art, including preferably derivatized Phe/Tyr/Trp amino acids, most preferably ring-substituted Phe/Tyr/Trp amino acids. Also encompassed are derivatives of Phe, Tyr and Trp, substituted by, e.g., a halogen, —CH₃, OH, —CH₂NH₃, —C(O)H, —CH₂CH₃, —CN, —CH₂CH₂CH₃, —SH, or another group. Exemplary non-natural aromatic amino acids include, but are not limited to, O-methyl-L-tyrosine; 3-methyl-phenylalanine; a p-acetyl-L-phenylalanine; O-4-allyl-L-tyrosine; 4-propyl-L-tyrosine; fluorinated phenylalanine; isopropyl-L-phenylalanine; a p-azido-L-phenylalanine; a p-acyl-L-phenylalanine; a p-benzoyl-L-phenylalanine; a phosphonotyrosine; a p-iodo-phenylalanine; p-bromophenylalanine; p-amino-L-phenylalanine; an isopropyl-L-phenylalanine; an amino-, isopropyl-, or O-allyl-containing phenylalanine analogue; a p-(propargyloxy) phenylalanine; 3-nitro-tyrosine; 5-fluoro-tryptophan, 5-hydroxy-tryptophan, 5-methoxy-tryptophan, 5-methyl-tryptophan, trifluoromethyl-tryptamine ethyl ester.

Methods for introducing non-natural amino-acids into proteins are also known in the art. For instance, non-naturally-occurring amino acids may be introduced by including synthetic aminoacyl-tRNAs in the IVTT system used to express the mutant monomer. Alternatively, they may be introduced by expressing the mutant monomer in E. coli that are auxotrophic for specific amino acids in the presence of synthetic (i.e. non-naturally-occurring) analogues of those specific amino acids. Non-natural amino acids may also be introduced using synthetic peptide chemistry methods known in the art for synthesizing peptides. Monomeric units of the nanopores may be formed entirely from synthetic peptides constructed using conjugation methods known in the art such as native chemical ligation (Thapa et al., Molecules, 2014, 14461-83), or cysteine coupling for example. Alternatively, monomers of the nanopore may comprise partially synthetic units coupled to naturally expressed peptide units using coupling methods known in the art.

The mutant nanopore may also be created by chemically attaching a suitable aromatic molecule to either the precursor monomeric units of the nanopore or the assembled oligomeric nanopore by means known in the art, such as for example by chemical attachment of suitable molecules to one or more cysteines (cysteine linkage), or lysines, which may either already exist in the wild-type protein or are introduced by mutagenesis.

In another aspect, the modified nanopore comprises a mutation to a natural aromatic amino acid, preferably one or more mutations to Trp, Tyr or Phe, more preferably Phe.

In addition to the aromatic amino acid mutation(s), one or more further mutation(s) may be introduced in the lumen-facing recognition region, which further mutation(s) increase the net negative charge of the pore.

For example, a mutant proteinaceous nanopore with mutation introduced to the lumen-facing recognition region to create a combination of aromatic residues that are “spatially adjacent” to negative residues, where the aromatic and negative residues are preferably within 4 nanometers, most preferably within 2 nanometers, of each other in a functional pore. The physical space between residue positions can be derived e.g. by measuring the C-alpha backbone distance between the respective mutated residues from a 3D model or crystal structure of an assembled oligomeric pore protein using common molecular modelling software known in the art.

Transmembrane protein pores, or fragments thereof, for use in accordance with the invention can be derived from beta-barrel pores or alpha-helix bundle pores. Beta-barrel pores comprise a barrel or channel that is formed from beta-strands. One skilled in the art would understand that a suitable nanopore of the invention may be selected from transmembrane pores that are known in the art (Peraro et al. Nat Rev Microbiol 2016; Crnković et al. Life (Basel) 2021). Suitable nanopores will ideally have dimensions in the recognition region most suitable for measuring small analytes, including small peptides. Suitable nanopores will ideally have transmembrane regions, recognition regions, or constrictions, with diameters of less than 2 nanometers, most preferably less than 1.5 nanometers. Suitable beta-barrel pores include, but are not limited to, beta-toxins, such as alpha-hemolysins, aerolysins, lysenin, cytolysins, cytolysin K, anthrax toxin and leukocidins, and outer membrane proteins/porins of bacteria, such as Mycobacterium smegmatis porin (Msp), for example MspA, MspB, MspC or MspD, outer membrane porin F (OmpF), outer membrane porin G (OmpG), outer membrane phospholipase A (OMPLA), ferric hydroxamate uptake component A (FhuA), Curli production transport component CsgG, and Neisseria autotransporter lipoprotein (NalP). Alpha-helix bundle pores comprise a barrel or channel that is formed from alpha-helices. Suitable alpha-helix bundle pores include, but are not limited to, inner membrane proteins and outer membrane proteins, such as Actinoporins, the outer membrane core complex (OMCC) of H. pylori Cag T4SS particles, and the transmembrane domain of the E. coli polysaccharide transporter Wza.

One skilled in the art would understand that a suitable nanopore of the invention may be an artificial nanopore, such as one adapted from known artificial nanopores. For example, nanopores based on trans-membrane beta-barrels or alpha helices attached to ring-like proteins (Zhang et al. BioRxiv 2020), proteins based on transmembrane peptides attached to DNA origami (Spruijt et al. Nat Nanotechnol 2018), self-assembling nanopores based on artificially created transmembrane peptides (Scott et al. Nat Chem 2021) and designed de novo (Vorobieva et al. Science 2021).

### Alpha-Helical Pores

In one aspect, the proteinaceous nanopore comprises a mutant actinoporin, or the alpha-helical transmembrane region (aa 1-27) thereof. For example, the mutant actinoporin comprises a mutation to an aromatic amino acid residue in the recognition region corresponding to amino acids 10-20. For homologs with additional N-terminal sequence, the region corresponding to the transmembrane alpha-helix can be determined by homology mapping and other means known in the art. The actinoporin family of pore-forming toxins is well known in the art. See for example Kristan et al. Toxicon 2009.

Exemplary members of the actinoporin family for preparing a mutant according to the present invention include Fragaceatoxin A (FraA), Fragaceatoxin B (FraB), Fragaceatoxin C (FraC), Fragaceatoxin D (FraD), Fragaceatoxin E (FraE), Equinatoxin II (Eqt-II), Equinatoxin IV (Eqt-IV), Equinatoxin V (Eqt-V), Urticinatoxin (UcI), Actitoxin-Oor1b (Or-G), Actitoxin-Oor1a (Or-A), Gigantoxin-4 (Gigt 4), Heteractis magnifica cytolysin III (HmgIII), Bandaporin (bp-1), Cribinopsis japonica toxin I (CJTOX I), Cribinopsis japonica toxin II (CJTOX II), Sticholysin I (StI), Sticholysin II (StII), Stichotoxin Hcr4a (RTX-A), Stichotoxin Hcr4b (RTX-SII) and Sagatoxin I (Src I).

The wildtype sequences and SwissProt accession numbers of exemplary alpha-helical pore forming proteins are as follows:

>Fragaceatoxin C|B9W5G6
(SEQ ID NO: 1)
SADVAGAVIDGAGLGFDVLKTVLEALGNVKRKIAVGIDNESGKTWT
AMNTYFRSGTSDIVLPHKVAHGKALLYNGQKNRGPVATGVVGVIAY
SMSDGNTLAVLFSVPYDYNWYSNWWNVRVYKGQKRADQRMYEELYY
HRSPFRGDNGWHSRGLGYGLKSRGFMNSSGHAILEIHVTKA
>Fragaceatoxin A|P0DUW8|
(SEQ ID NO: 2)
SAEVAGAVIEGAKLTFNVLQ
>Fragaceatoxin B|A0A515MEN7
(SEQ ID NO: 3)
SLTFDVLQTVLKALGDVSRKIAVGIDNEPGMTWTAMNTYFRSGTSD
VILPHTVPHSKALLYDGQKNRGPVTTGVVGVIAYAMSDGNTLAVLF
SIPFDYNLYSNWWNVKVYKGHRRADQAMYEELYYDFSPFRGDNGWH
TKSIGYGLKGRGFMNSSGKAILQIHVNKV
>Fragaceatoxin D|P0DUW9
(SEQ ID NO: 4)
SVAVAGAVIKGAALTFNILQ
>Fragaceatoxin E|A0A515MEM9
(SEQ ID NO: 5)
AGLGFDVLKTVLEALGNVKRKIAVGIDNESGRTWTAMNTYFRSGTS
DIVLPHKVAHGKALLYNGQKNRGPVATGVVGVIAYSMSDGNTLAVL
FSVPYDYNWYSNWWNVRVYKGQKRANQRMYEELYYHRSPFRGDNGW
HSRSLGYGLKSRGFMNSSGHAILEIHVTKA
>Equinatoxin II|P61914
(SEQ ID NO: 6)
SADVAGAVIDGASLSFDILKTVLEALGNVKRKIAVGVDNESGKTWT
ALNTYFRSGTSDIVLPHKVPHGKALLYNGQKDRGPVATGAVGVLAY
LMSDGNTLAVLFSVPYDYNWYSNWWNVRIYKGKRRADQRMYEELYY
NLSPFRGDNGWHTRNLGYGLKSRGFMNSSGHAILEIHVSKA
>Equinatoxin IV|Q9Y1U9
(SEQ ID NO: 7)
SVAVAGAIIKGAALTFNVLQTVLKALGDISRKIAVGVDNESGKTWT
ALNTYFRSGTSDIVLPHKVPHGKALLYNGQKDRGPVATGAVGVLAY
AMSDGNTLAVLFSVPYDYNWYSNWWNVRIFKGRRRADQRMYEQLYY
YLSPFRGDNGWHERHLGYGLKSRGFMNSGGQAILEIHVTKA
>Equinatoxin V|Q93109
(SEQ ID NO: 8)
SVAVAGAVIEGATLTFNVLQTVLKALGDISRKIAVGIDNESGMTWT
AMNTYFRSGTSDVILPHTVPHGKALLYNGQKDRGPVATGVVGVLAY
AMSDGNTLAVLFSIPFDYNLYSNWWNVKVYKGHRRADQRMYEELYY
NLSPFRGDNGWHNRDLGYGLKGRGFMNSSGQSILEIHVTKA
>Urticinatoxin|C9EIC7
(SEQ ID NO: 9)
SVAIAGAVIEGAKLTFGILEKILTVLGDINRKIAIGVDNESGREWT
AQNAYFFSGTSDVVLPASVPNTKAFLYNAQKDRGPVATGVVGVLAY
SLSNGNTLGILFSVPYDYNLYSNWWNIKLYKGIKRADRDMYNDLYY
YAHPHKGDNGWHENSLGFGLKSKGFMTSSGQTILQIRVSRA
>OrG|Q5I2B1
(SEQ ID NO: 10)
GAIIAGAALGFNVHQTVLKALGQVSRKIAIGVDNESGGTWTALNAY
FRSGTTDVILPEFVPNQKALLYSGQKDTGPVATGAVGVLAYYMSDG
NTLGVMFSVPFDYNLYSNWWDVKVYRGRRRADQAMYEGLLYGIPYG
GDNGWHARKLGYGLKGRGFMKSSAQSILEIHVTKA
>OrA|Q5I4B8
(SEQ ID NO: 11)
ATFRVLAKVLAELGKVSRKIAVGVDNESGGSWTALNAYFRSGTTDV
ILPDLVPNQKALLYRGGKDTGPVATGVVGVLAYAMSDGNTLAILFS
VPYDYNLYSNWWNVKVYSGKRRADQGMSEDLSYGNPYGGDNGWHAR
KLAYGLKERGFMKSSAQSILEIHATKA
>Gigantoxin4|H9CNF5
(SEQ ID NO: 12)
ASAVAGTIIEGASLTFQILDKVLTELGNVSRKIAIGIDNESGGSWT
AMNAYFRSGTTDVILPEFVPNNKALLYSGRKDTGPVTTGAVGALAY
YMSDGNTLAVMFSVPFDYNLYSNWWDVRVYSGKRRADQKMYEDLYN
GSPFKGDNGWHQKNLGYGLRMKGIMTSAGEAKLQIKISR
>HmgIII|Q9U6X1
(SEQ ID NO: 13)
SAALAGTIIEGASLGFQILDKVLGELGKVSRKIAVGVDNESGGSWT
ALNAYFRSGTTDVILPEFVPNQKALLYSGRKDTGPVATGAVAAFAY
YMSNGHTLGVMFSVPFDYNFYSNWWDVKVYSGKRRADQGMYEDMYY
GNPYRGDNGWHQKNLGYGLRMKGIMTSAGEAILQIRISR
>Bandaporin|C5NSL2
(SEQ ID NO: 14)
SLAVAGAVIEGGNLVMSVLDRILEAIGDVNRKIAIGVENQSGKSWT
AMNTYFRSGTSDVVLPHSVPSGKALLYDGQKTRGPVATGVVGVFAY
AMSDGNTLAVMFSIPYDYNLYSNWWNVKTYSGMKRADQSMYEDLYY
HASPFKGDNGWHSRNLGYGLKCRGFMNSSGAAKLEIHVSRA
>CJTOX I|A0A2Z5Z9X0
(SEQ ID NO: 15)
LPMKEDISNEERPTSVNEKPVKKSVAVAGAVIQGAALAFQVLDKIL
TSLGGIGRKIAIGVDNESGMKWAARNVYFYSGTSDTVLPYSVPHSK
AFLYGARKTRGSVRGAVGVLAYSMSDGNTLGILFSVPYDYNWYSNW
WNIKVYRGYKRANKWMYHDLYYYARPHKGNNEWHEKSLGYGLKSKG
FMTSSGQTKLEIRVSRA
>CJTOX II|A0A2Z5Z9H5
(SEQ ID NO: 16)
LPMKEDISNDERPISVNEEPVKKNAAVAGAVIQGATLTFQVLDRIL
TVLGDISRKIAIGVDNESGRKWTAKNAYFFSGTSDVVLPYSVPNGK
AFLYDGKKTRGPVATGAVGVLAYSMSDGNTLGILFSVPYDYNWYEN
WWNIKVYSGSKRANKWMYENLYYNASPHKGDNGWHEKSLGYGLKSR
GYMASSGQTKLEIRVTRA
>Sticholysin I|P81662
(SEQ ID NO: 17)
SELAGTIIDGASLTFEVLDKVLGELGKVSRKIAVGIDNESGGTWTA
LNAYFRSGTTDVILPEVVPNTKALLYSGRKSSGPVATGAVAAFAYY
MSNGNTLGVMFSVPFDYNWYSNWWDVKIYPGKRRADQGMYEDMYYG
NPYRGDNGWYQKNLGYGLRMKGIMTSAGEAKMQIKISR
>Sticholysin II|P07845
(SEQ ID NO: 18)
ALAGTIIAGASLTFQVLDKVLEELGKVSRKIAVGIDNESGGTWTAL
NAYFRSGTTDVILPEFVPNTKALLYSGRKDTGPVATGAVAAFAYYM
SSGNTLGVMFSVPFDYNWYSNWWDVKIYSGKRRADQGMYEDLYYGN
PYRGDNGWHEKNLGYGLRMKGIMTSAGEAKMQIKISR
>RTXA|P58691
(SEQ ID NO: 19)
ALAGAIIAGASLTFQILDKVLAELGQVSRKIAIGIDNESGGSWTAM
NAYFRSGTTDVILPEFVPNQKALLYSGRKNRGPDTTGAVGALAYYM
SNGNTLGVMFSVPFDYNLYSNWWDVKVYSGKRRADQAMYEDLYYSN
PYRGDNGWHQKNLGYGLKMKGIMTSAGEAIMEIRISR
>RTXSII|P0C1F8
(SEQ ID NO: 20)
SAALAGTITLGASLGFQILDKVLGELGKVSRKIAVGVDNESGGSWT
ALNAYFRSGTTDVILPEFVPNQKALLYSGRKDTGPVATGAVAAFAY
YMSNGHTLGVMFSVPFDYNLYSNWWDVKIYSGKRRADQAMYEDMYY
GNPYRGDNGWHQKNLGYGLKMKGIMTSAVEAILEIRISR
>Src I|Q86FQ0
(SEQ ID NO: 21)
KISGGTVIAAGRLTLDLLKTLLGTLGSISRKIAIGVDNETGGLITG
NNVYFRSGTSDDILPHRVETGEALLYTARKTKGPVATGAVGVFTYY
LSDGNTLAVLFSVPFDYNFYSNWWNVKIYSGKRNADYDMYHELYYD
ANPFEGDDTWEYRYLGYGMRMEGYMNSPGEAILKITVMPD
>Cytolysin AvtI|Q5R231
(SEQ ID NO: 22)
SAAVAGAVIAGGELALKILTKILDEIGKIDRKIAIGVDNESGLKWT
ALNTYYKSGASDVTLPYEVENSKALLYTARKSKGPVARGAVGVLAY
KMSSGNTLAVMFSVPFDYNLYSNWWNVKIYDGEKKADEKMYNELYN
NNNPIKPSTWEKRDLGKDGLKLRGFMTSNGDAKLVIHIEKS
>Cytolysin PsTX20A|P0DL55
(SEQ ID NO: 23)
SAAVAGAVIAGGELALKILTKILDEIGKIDRKIAIGVDNESGLKWT
ALNTYYKSGASDVTLPYEVENSKALLYTARKSKGPVARGAVGVLAY
KMSSGNTLAVMFSVPFDYNLYTNWWNVKIYDGEKKADEKMYNELYN
NNNPIKPSIWEKRDLGQDGLKLRGFMTSNGDAKLVIHIEKS
>Nigrelysin|A0A345GPN1
(SEQ ID NO: 24)
LPLEEKEDEKDEKRSLEVAGAVMEGANLGMSVLQTILQAIGDVSRK
IAVGVDNESGRSWTAQNAYFRSGTSDVILPHTVPSGKALLYDGQKN
RGPVATGVVGVITYTMGDGNTLAVMFSVPYDYNWYSNWWNVKIYHG
KVRASQKMYEDLYYYRSPFKGDNGWHERNLGYGLKSKGFMNSSGAA
LLQIKVMKA

Also encompassed are pore-forming toxin homologs thereof showing at least 80%, at least 85%, at least 90%, or at least 95% sequence identity with any of these family members, provided that the pore-forming toxin retains the ability to create oligomeric nanopores in membranes. This functionality can be readily tested by in vitro using methods known in the art. For example, putative purified nanopores can be inserted into model membranes as described herein or using other means known in the art (e.g. vesicle insertion, detergent insertion, spontaneous insertion, etc) and characterized by electrophysiology means to determine their ability to pass ionic current and detect the presence of model analytes added to the system.

As will be understood by a person skilled in the art, the amino acid sequence of a given family member will contain one or more mutation(s) in the recognition region of the pore. Thus, if reference is made to e.g. a pore comprising FraD, Eqt-IV or StII, this is meant to refer to FraD, Eqt-IV or StII mutants of which the internal degree of aromaticity has been manipulated, optionally in combination with the introduction of negatively charged residue(s), in accordance with the present invention.

For example, a proteinaceous nanopore according to the invention advantageously comprises a mutant actinoporin selected from the group consisting of:
(i) FraC, FraE or a functional homolog showing at least 90% sequence identity therewith, comprising an aromatic residue at the position corresponding to Gly13 of FraC;
(ii) FraB, Ten-C, Eqt-II, Gigt 4, HmgII, RTX-SII, Hmt, or a functional homolog showing at least 90% sequence identity therewith, comprising an aromatic residue at the position corresponding to Ser13, and optionally comprising an acidic residue at position 10;
(iii) bp-1 or a functional homolog showing at least 90% sequence identity therewith, comprising an aromatic residue at the position corresponding to Asn13;
(iv) CJTOX I or a functional homolog showing at least 90% sequence identity therewith, comprising an aromatic residue at the position corresponding to Thr36, and optionally comprising an acidic residue at the position corresponding to Gln33;
(v) CJTOX II or a functional homolog showing at least 90% sequence identity therewith, comprising an aromatic residue at the position corresponding to Ala36, and optionally comprising an acidic residue at the position corresponding to Gln33;
(vi) Cytolysin Avt-I, Cytolysin PsTX-20A or a functional homolog showing at least 90% sequence identity therewith, comprising an aromatic residue at the position corresponding to Glu13, and optionally comprising an acidic residue at the position corresponding to Ala 10;
(vii) Eqt-IV or a functional homolog showing at least 90% sequence identity therewith, comprising an aromatic residue at the position corresponding to Ala13 and optionally comprising an acidic residue at the position corresponding to Lys10;
(viii) Eqt-V or a functional homolog showing at least 90% sequence identity therewith, comprising an aromatic residue at the position corresponding to Thr13;
(ix) Nigrelysin or a functional homolog showing at least 90% sequence identity therewith, comprising an aromatic residue at the position corresponding to Asn27;
(x) StII, RTX-A or a functional homolog showing at least 90% sequence identity therewith, comprising an aromatic residue at the position corresponding to Ser11, and optionally comprising an acidic residue at the position corresponding to Ala8;
(xi) Src I or a functional homolog showing at least 90% sequence identity therewith, comprising an aromatic residue at the position corresponding to Arg12, and optionally comprising an acidic residue at the position corresponding to Ala9;
(xii) StI or a functional homolog showing at least 90% sequence identity therewith, comprising an aromatic residue at the position corresponding to Ser12;
(xiii) UcI or a functional homolog showing at least 90% sequence identity therewith, comprising an aromatic residue at the position corresponding to Lys13; or
(xiv) Or-G or a functional homolog showing at least 90% sequence identity therewith, comprising an aromatic residue at the position corresponding to Ala8, and optionally comprising an acidic residue at the position corresponding to Ala5; or the alpha-helical transmembrane region (aa 1-27) thereof including the recited mutation(s).

In one embodiment, the mutant pore-forming toxin or pore-forming alpha-helical fragment is selected from Table 1, depicting the one or more specific aromatic mutations in each of the actinoporins that are equivalent to the mutations of the lumen-facing residues at positions 10, 13, 17 and 20 of FraC.

Also encompassed are pore-forming toxin homologs comprising the defined mutation(s) and showing at least 85%, at least 87%, at least 90%, at least 93%, at least 95% or at least 98% sequence identity.

Very good results can be obtained with a proteinaceous nanopore comprising mutant FraC or a pore-forming alpha-helical fragment thereof, comprising mutation Gly13Tyr, Gly13Trp or Gly13Phe, preferably Gly13Phe.

TABLE 1

| Pore forming | | | | | |
|---|---|---|---|---|---|
| toxin | SwissProt | | | | |
| FraC | B9W5G6 | D10W/F/Y | G13W/F/Y | D17W/F/Y | K20W/F/Y |
| FraA | P0DUW8 | E10W/F/Y | K13W/F/Y | N17W/F/Y | Q20W/F/Y |
| FraB | A0A515MEN7 | K12W/F/Y | G15W/F/Y | R19W/F/Y | A22W/F/Y |
| FraD | P0DUW9 | K10W/F/Y | A13W/F/Y | N17W/F/Y | Q20W/F/Y |
| FraE | A0A515MEM9 | E13W/F/Y | G16W/F/Y | R20W/F/Y | A22W/F/Y |
| Eqt-II | P61914 | D10W/F/Y | S13W/F/Y | D17W/F/Y | K20W/F/Y |
| Eqt-IV | Q9Y1U9 | K10W/F/Y | A13W/F/Y | N17W/F/Y | Q20W/F/Y |
| Eqt-V | Q93109 | E10W/F/Y | T13W/F/Y | N17W/F/Y | Q20W/F/Y |
| UcI | C9EIC7 | E10W/F/Y | K13W/F/Y | G17W/F/Y | E20W/F/Y |
| Or-G | Q5I2B1 | K20W/F/Y | G23W/F/Y | R27W/F/Y | A30W/F/Y |
| Or-A | Q5I4B8 | E12W/F/Y | K15W/F/Y | K19W/F/Y | V22W/F/Y |
| Gigt 4 | H9CNF5 | E10W/F/Y | S13W/F/Y | Q17W/F/Y | D20W/F/Y |
| HmgIII | Q9U6X1 | E10W/F/Y | S13W/F/Y | Q17W/F/Y | D20W/F/Y |
| bp-1 | C5NSL2 | E10W/F/Y | N13W/F/Y | S17W/F/Y | D20W/F/Y |
| CJTOX I | A0A2Z5Z9X0 | Q33W/F/Y | T36W/F/Y | Q40W/F/Y | D43W/F/Y |
| CJTOX II | A0A2Z5Z9H5 | Q33W/F/Y | A36W/F/Y | Q40W/F/Y | D43W/F/Y |
| StI | P81662 | A8W/F/Y | S11W/F/Y | E15W/F/Y | D18W/F/Y |
| StII | P07845 | D9W/F/Y | E12W/F/Y | Q16W/F/Y | D19W/F/Y |
| RTX-A | P58691 | A8W/F/Y | E11W/F/Y | Q15W/F/Y | D18W/F/Y |
| RTX-SII | P0C1F8 | L10W/F/Y | S13W/F/Y | Q17W/F/Y | D20W/F/Y |
| Src I | Q86FQ0 | A9W/F/Y | R12W/F/Y | D16W/F/Y | K19W/F/Y |
| AvtI | Q5R231 | A10W/F/Y | E13W/F/Y | K17W/F/Y | T20W/F/Y |
| PsTX20A | P0DL55 | A10W/F/Y | E13W/F/Y | K17W/F/Y | T20W/F/Y |
| Nigr | A0A345GPN1 | E24W/F/Y | N27W/F/Y | S31W/F/Y | D34W/F/Y |

### Beta-Barrel Pores

In another aspect, the invention relates to a proteinaceous nanopore comprising a mutant beta-barrel pore-forming protein, or a pore-forming fragment thereof, wherein the lumen-facing recognition region of the pore-forming protein or fragment thereof comprises one or more mutations of lumen-facing non-aromatic residue(s) to a natural or non-natural aromatic amino acid residue, preferably one or more mutations to Trp, Tyr or Phe.

Preferably, the beta-barrel pore-forming toxin has an internal diameter (pore size; constriction) in the recognition region in the range of 0.2 to 2.0 nanometers, most preferably a minimum internal diameter of 0.5 to 1.5 nanometers. For example, it is selected from the group consisting of alpha-hemolysin, aerolysin, lysenin, epsilon-toxin (ETX), hemolytic lectin (LSL), cytolysin k (cytK), and functional homologs showing at least 80%, preferably at least 85%, more preferably at least 90% sequence identity therewith. Preferably, it is selected from the group consisting of aerolysin, lysenin and cytolysin k (cytK), and functional homologs showing at least 90%, preferably at least 95%, more preferably at least 98% sequence identity therewith.

Similar to what is described herein above for the alpha-helical type pore forming proteins, the pore may further comprise one or more mutations increasing the net negative charge or decreasing the net positive charge of the barrel or channel of the pore, with the aim of increasing the flux of cations through the nanopore (Table 4), especially under acidic pH conditions (pH<4.5).

The wildtype sequences and SwissProt accession numbers of exemplary beta-barrel pore forming proteins are as follows:

>Aerolysin|P09167
(SEQ ID NO: 25)
MQKIKLTGLSLIISGLLMAQAQAAEPVYPDQLRLFSLGQGVCGDKY
RPVNREEAQSVKSNIVGMMGQWQISGLANGWVIMGPGYNGEIKPGT
ASNTWCYPTNPVTGEIPTLSALDIPDGDEVDVQWRLVHDSANFIKP
TSYLAHYLGYAWVGGNHSQYVGEDMDVTRDGDGWVIRGNNDGGCDG
YRCGDKTAIKVSNFAYNLDPDSFKHGDVTQSDRQLVKTVVGWAVND
SDTPQSGYDVTLRYDTATNWSKINTYGLSEKVTTKNKFKWPLVGET
ELSIEIAANQSWASQNGGSTTTSLSQSVRPTVPARSKIPVKIELYK
ADISYPYEFKADVSYDLTLSGFLRWGGNAWYTHPDNRPNWNHTFVI
GPYKDKASSIRYQWDKRYIPGEVKWWDWNWTIQQNGLSTMQNNLAR
VLRPVRAGITGDFSAESQFAGNIEIGAPVPLAADSKVRRARSVDGA
GQGLRLEIPLDAQELSGLGFNNVSLSVTPAANQ
>CytK|Q937V2
(SEQ ID NO: 26)
MKRSKTYLKCLALSAVFASSALALSTPAAYAQTTSQVVTDIGQNAK
THTSYNTFNNDQTDNMTMSLKVTFIDDPSADKQIAVINTTGSFLKA
NPTISSAPIDNYPIPGASATLRYPSQYDIAFNLQDNSARFFNVAPT
NAVEETTVTSSVSYQLGGSVKASATPNGLSAEAGATGQVTWSDSVS
YKQTSYKTNLIDQTNKNVKWNVFFNGYNNQNWGIYTRDSYHSLYGN
QLFMYSRTYLYESDAKGNLIPMDQLPALTNSGFSPGMIAVVISEKN
TDQSNLQVAYTKHADDYQLRPGFTFGTANWVGNNVKDVDQKTFNKS
FTLDWKNKKLVEKNR
>Alpha-hemolysin|P09616.2
(SEQ ID NO: 27)
MKTRIVSSVTTTLLLGSILMNPVAGAADSDINIKTGTTDIGSNTTV
KTGDLVTYDKENGMHKKVFYSFIDDKNHNKKLLVIRTKGTIAGQYR
VYSEEGANKSGLAWPSAFKVQLQLPDNEVAQISDYYPRNSIDTKEY
MSTLTYGFNGNVTGDDTGKIGGLIGANVSIGHTLKYVQPDFKTILE
SPTDKKVGWKVIFNNMVNQNWGPYDRDSWNPVYGNQLFMKTRNGSM
KAADNFLDPNKASSLLSSGFSPDFATVITMDRKASKQQTNIDVIYE
RVRDDYQLHWTSTNWKGTNTKDKWTDRSSERYKIDWEKEEM
>Gamma-hemolysin component B|P0A075.1
(SEQ ID NO: 28)
MNKLVKSSVATSMALLLLSGTANAEGKITPVSVKKVDDKVTLYKTT
ATADSDKFKISQILTFNFIKDKSYDKDTLVLKATGNINSGFVKPNP
NDYDFSKLYWGAKYNVSISSQSNDSVNVVDYAPKNQNEEFQVQNTL
GYTFGGDISISNGLSGGLNGNTAFSETINYKQESYRTTLSRNTNYK
NVGWGVEAHKIMNNGWGPYGRDSFHPTYGNELFLAGRQSSAYAGQN
FIAQHQMPLLSRSNFNPEFLSVLSHRQDGAKKSKITVTYQREMDLY
QIRWNGFYWAGANYKNFKTRTFKSTYEIDWENHKV
>Leukocidin-F|P31715.2
(SEQ ID NO: 29)
MNKLVKSSVATSMALLLLSGTANAEGKITPVSVKKVDDKVTLYKTT
ATADSDKFKISQILTFNFIKDKSYDKDTLVLKATGNINSGFVKPNP
NDYDFSKLYWGAKYNVSISSQSNDSVNAVDYAPKNQNEEFQVQNTL
GYTFGGDISISNGLSGGLNGNTAFSETINYKQESYRTLSRNTNYKN
VGWGVEAHKIMNGWGPYGRDSFHPTYGNELFLAGRQSSAYAGQNFI
AQHQMPLLSRSNFNPEFLSVLSHRQDRAKKSKITVTYQREMDLYQI
RWNGFYWAGANYKNFKTRTFKSTYEIDWENHKV
>Leucotoxin LukD|O54082.1
(SEQ ID NO: 30)
IEKLGKSSVASSIALLLLSNTVDAAQNITPKREKKVDDKITLYKTT
ATSDNDKLNIFQILTFNFIKDKSYDKDTLVLKAAGNINSGYKNSNP
KDYNYSQFYWGGKYNVSVSSESNDAVNVVDYAPKNQNEEFQVQQTL
GYSYGGDINISNGLSGGLNGSKSFSETINYKQESYRTTIDRKTNHK
SIGWGVEAHKIMNNGWGPYGRDSYDPTYGNELFLGGDKSSSNAGQN
FLPTHQIPLLARGNFNPEFISVLSHKLFDTKKSKIKVTYQREMDRY
TNQWNRSHWVGNNYKNQNTVTFTSTYEVDWQN
>Gamma-hemolysin component A|P0A071.1
(SEQ ID NO: 31)
IKNKILTATLAVGLIAPLANPFIEISKAENKIEDIGQGAEIIKRTQ
DITSKRLAITQNIQFDFVKDKKYNKDALVVKMQGFISSRTTYSDLK
KYPYIKRMIWPFQYNISLKTKDSNVDLINYLPKNKIDSADVSQKLG
YNIGGNFQSAPSIGGSGSFNYSKTISYNQKNYVTEVESQNSKGVKW
GVKANSFVTPNGQVSAYDQYLFAQDPTGPAARDYFVPDNQLPPLIQ
SGFNPSFITTLSHERGKGDKSEFEITYGRNMD
>Leukocidin-S subunit|P31716.1
(SEQ ID NO: 32)
DIGKGSDIEIIKRTEDKTSNKWGVTQNIQFDFVKDTKYNKDALILK
MQGFISSRTTYYNYKKTNHVKAMRWPFQYNIGLKTNDKYVSLINYL
PKNKIESTNVSQTLGYNIGGNFQSAPSLGGNGSFNYSKSISYTQQN
YVSEVEQQNSKSVLWGVKANSFATESGQKSAFDSDLFVGYKPHSKD
PRDYFVPDSELPPLVQSGFNPSFIATVSHEKGSSDTSEFEITYGRN
MDVTHAIKRSTHYGNSYLDGHRVHNAFVNRNYTVKYEVNWKTHEI
>Gamma-hemolysin component C|Q5HDD4.1
(SEQ ID NO: 33)
DIGKGSDIEIIKRTEDKTSNKWGVTQNIQFDFVKDKKYNKDALILK
MQGFISSRTTYYNYKKTNHVKAMRWPFQYNIGLKTNDKYVSLINYL
PKNKIESTNVSQILGYNIGGNFQSAPSLGGNGSFNYSKSISYTQQN
YVSEVEQQNSKSVLWGVKANSFATESGQKSAFDSDLFVGYKPHSKD
PRDYFVPDSELPPLVQSGFNPSFIATVSHEKGSSDTSEFEITYGRN
MDVTHAIKRSTHYGNSYLDGHRVHNAFVNRNYTVKYEVNWKTHEI
>Leucotoxin LukEv|Q2FXB0.2
(SEQ ID NO: 34)
VTQNVQFDFVKDKKYNKDALIVKMQGFINSRTSFSDVKGSGYELTK
RMIWPFQYNIGLTTKDPNVSLINYLPKNKIETTDVGQTLGYNIGGN
FQSAPSIGGNGSFNYSKTISYTQKSYVSEVDKQNSKSVKWGVKANE
FVTPDGKKSAHDRYLFVQSPNGPTGSAREYFAPDNQLPPLVQSGFN
PSFITTLSHEKGSSDTSEFEISYGRNLD
>Lysenin Lys|O18423
(SEQ ID NO: 35)
MSAKAAEGYEQIEVDVVAVWKEGYVYENRGSTSVDQKITITKGMKN
VNSETRTVTATHSIGSTISTGDAFEIGSVEVSYSHSHEESQVSMTE
TEVYESKVIEHTITIPPTSKFTRWQLNADVGGADIEYMYLIDEVTP
IGGTQSIPQVITSRAKIIVGRQIILGKTEIRIKHAERKEYMTVVSR
KSWPAATLGHSKLFKFVLYEDWGGFRIKTLNTMYSGYEYAYSSDQG
GIYFDQGTDNPKQRWAINKSLPLRHGDVVTFMNKYFTRSGLCYDDG
PATNVYCLDKREDKWILEVV

The following list indicates lumen-facing amino acid position(s) in exemplary beta-barrel pores to be mutated into an aromatic residue.
> Aerolysin (pdb: 5jzt, uniprot: UniProtKB—P09167): 222, 224, 226, 228, 230, 232, 236, 234, 238, 240, 242, 244, 246, 252, 253, 254, 256, 258, 260, 262, 264, 266, 268, 272, 270, 274
> Alpha-hemolysin (pdb: 7ahl, uniprot: UniProtKB—P09616):109, 111, 113, 115, 117, 121, 119, 123, 125, 127, 129, 131, 135, 133, 139, 137, 141, 145, 143, 147
>NetB (pdb: 4h56, uniprot: UniProtKB—A8ULG6): 114, 116, 118, 120, 124, 122, 128, 126, 130, 132, 135, 141, 143, 145, 147, 149, 151
>HlyA (pdb: 3o44, uniprot: UniProtKB—P09545): 279, 281, 283, 285, 289, 287, 293, 291, 297, 295, 299, 301, 304, 306, 308, 310, 312, 314, 316, 318, 320
>Hemolytic lectin (pdb: 3w9t, uniprot: UniProtKB—Q868M7): 305, 307, 309, 311, 313, 315, 317, 319, 323, 321, 325, 327, 329, 331, 338, 340, 342, 344, 346, 348, 350, 352, 354, 356, 358, 360, 362
>Bacillus protective antigen (pdb: 3j9c); 278, 280, 282, 284, 286, 288, 290, 292, 294, 296, 298, 300, 302, 304, 306, 308, 310, 312, 313, 314, 315, 319, 317, 321, 325, 323, 327, 329, 331, 333, 335, 337, 339, 341, 343, 345, 347
>Lysenin (pdb: 5gaq, uniprot: UniProtKB—P13423): 35, 37, 39, 41, 45, 43, 47, 49, 51, 53, 55, 57, 59, 63, 61, 65, 68, 74, 76, 78, 80, 82, 84, 86, 88, 90, 92, 94, 96, 98, 100, 102, 104
>Epsilon-toxin B (pdb: 6rb9, uniprot: UniProtKB—Q02307): 99, 101, 104, 106, 108, 110, 112, 114, 116, 118, 120, 124, 122, 126, 128, 130, 132, 136, 137, 141, 139, 143, 145, 147, 149, 151, 153, 155, 157, 159, 161, 163, 165, 166, 168

In one aspect, the invention provides a mutant proteinaceous nanopore comprising a mutant of the aerolysin-like 3-PFP (ap-PFP) subfamily.

For example, it provides aerolysin (Aer) comprising an aromatic amino acid substitution in the water-facing region of the pore, which region runs from residues 212 to 242 and from 256 to 284. In one aspect, the aromatic mutation is at least in the region 212-242. For example, one or more basic residue(s) is/are replaced with an aromatic residue in order to reduce the net positive charge. In some embodiments the mutant is either W, Y or F substituted at one of more positions including Q212, G214, D216, T218, R220, D222, A224, N226, S228, T230, T232, G234, S236, K238, T240 or K242 This may comprise substituting K238 with W, Y or F. In a specific embodiment, the mutant is Aer-K238F or Aer-K238W.

In another aspect, the aromatic mutation is at least in the region 256-284. For example, the position corresponding to S256, E258, A260, N262, S264, A266, Q268, G270, S272, T274, S276, S278, S280, R282 and/or T284 is mutated to Trp, Tyr or Phe. The aromatic substitution is preferably combined with an acidic substitution, for example at position K238. In one embodiment, mutant aerolysin comprises mutation K238D. In a specific aspect, the nanopore comprises aerolysin mutant A260F, S264F, Q268F, S272F, and/or comprising mutation K238D. Preferred mutants include Aer-K238D, Aer-K238D-A260F, Aer-K238D-S264F, Aer-K238D-Q268F and Aer-K238D-S272F. These mutant pores are suitably used for analyte detection, preferably (unlabeled) peptide detection, at a pH≤4.5, e.g pH 3.8 or pH 3.0. See example 7 herein below.

In another embodiment, the invention provides a mutant proteinaceous nanopore comprising a mutant Lysenin or a pore-forming fragment thereof. The lysenin pore resembles the mushroom-shaped pore complexes of the α-haemolysin family of small (3-PFTs, although structures of their water-soluble monomers are fundamentally different. In one specific embodiment, the Lys mutant comprises an aromatic substitution at position Glu76, for example it is mutant Lys-E76F.

In another aspect, the invention provides a mutant proteinaceous nanopore comprising a mutant Cytotoxin K (CytK) or a pore-forming fragment thereof. CytK is a pore-forming toxin of Bacillus cereus (Hardy et al. FEMSMicrobiolLett. 2001). Although confirmed to be a nanopore with suitable properties for sensing, as of to date no structure exists for the CytK nanopore to aid mutagenesis. A person of skill in the art will understand that the transmembrane beta-barrel region of the protein, and therefore the putative recognition region for sensing analytes, can be determined by homology modelling to a suitably similar structure (e.g. alpha-hemolysin from S. aureus), and then confirmed experimentally by mutagenesis in the candidate region. Exemplary CytK mutants of the invention comprise an aromatic amino acid substitution in the lumen-facing region of the pore, which region runs from residue 112 to residue 155. Residues identified as most suitable for aromatic substitution, and optionally in combination with one or more acidic substitutions, include one or more (e.g. up to 5, preferably up to 4, more preferably up to 3 or 2) of the following lumen-facing non-aromatic transmembrane residues: E112/T114/T116/S118/S120/Q122/G124/S126/K128/S130/T132/S134/S137/E139/G141/T 143/Q145/T147/S149/S151/S153/K155, preferably one or more of E112/T114/T116/S118/S120/Q122/G124/S126.

These aromatic mutations are advantageously combined with one or more acidic substitutions of neutral and/or positively charged residue(s), such as K128E or K128D, or any of the lumen-facing residues that can be substituted with an aromatic residue, excluding those already negative. Such mutations ideally make the barrel of the pore net negative (if not already so), thereby altering the electro-osmotic flow through the pore.

In a specific aspect, the mutant pore or fragment thereof comprises a CytK mutant with an aromatic amino acid at position S126 or K128, for example comprising mutation Ser126Tyr, Ser126Trp, Ser126Phe, Lys128Tyr, Lys128Trp, Lys128Phe, preferably Ser126Phe or Lys128Phe.

In another specific aspect, the mutant pore or fragment thereof comprises a CytK mutant with an aromatic amino acid substitution higher “up” in the barrel, for instance at position Ser120, Gln122 or Gly124. See FIG. 20. Exemplary cytK mutant nanopores according to the invention comprise mutation S120W/F/Y+K128D, Q122W/F/Y+K128D, G124W/F/Y+K128D or S126W/F/Y+K128D.

Similar to what is described herein above for the alpha-helical type pore forming protein and the beta barrel pore forming protein, the pore may further comprise one or more mutations increasing the net negative charge or decreasing the positive charge of the barrel or channel of the pore, with the aim of increasing the flux of cations through the nanopore (Table 3), especially under acidic pH conditions (pH≤4.5). As shown for the mutation K128F or K128D (FIGS. 19 and 20).

In a specific aspect, as found for the pore forming proteins described above, the mutant pore comprises a CytK mutant with an aromatic amino acid in the water facing region of the nanopore, for example S126F, further comprising a mutation to increase the negative charge of the water facing region, for example K128D, which improves the analysis of peptides.

### Analytical System

A further aspect of the invention relates to an analytical system comprising a mutant proteinaceous nanopore according to the invention. For example, the analytical system comprises a hydrophobic membrane separating a fluid chamber into a cis side and a trans side, wherein the mutant proteinaceous nanopore is inserted in said membrane. A nanopore sensor system may comprise: i) a fluid-filled compartment separated by a membrane into a first cis chamber and a second trans chamber, wherein the fluid is an ionic solution; ii) an engineered mutant pore of the invention inserted in the membrane; and iii) electrodes configured for measuring an ionic current flow through the nanopore and optionally generating an electrical potential difference across the membrane to facilitate ionic flow through the pore from the first chamber to the second chamber and vice versa.

Herewith, the system provides a pore-based sensor. In one specific aspect, the analytical system comprises a mutant alpha-helical pore forming toxin of the actinoporin family, preferably FraC. In another specific aspect, the analytical system comprises a mutant beta-barrel pore forming toxin, preferably an aerolysin-like OPFP or cytotoxin K.

When a system according to the invention is in use, the nanopore is typically positioned between a first liquid medium and a second liquid medium, wherein at least one liquid medium comprises an analyte of interest, and wherein the system is operative to detect a property of the analyte. In one embodiment, the system is operative to detect a property of the analyte by subjecting the nanopore to an electric field such that the analyte electrophoretically and/or electroosmotically translocates through the nanopore.

As exemplified herein below, a system provided herein is particularly suitable for the analysis of a proteinaceous substance, preferably a peptide, more preferably a peptide up to about 30 amino acids in length. More in particular, a system of the invention provides for capture of peptides up to 20, 15, 10, 5, 3 or 2 amino acids in length. As is exemplified herein below, a mutant nanopore can detect peptide(s) with a highly variable amino acid composition. Hence, it can be broadly applied without restriction to any specific structure and/or property. However, in one aspect, the peptide comprises at least 50%, preferably 60%, more preferably at least 70% of hydrophobic and charged amino acids. For example, it contains up to 40%, preferably up to 30%, more preferably up to 25% of aromatic amino acids (Tyr, Trp and Phe).

However, this is in no way to be understood that the invention is limited to applications relating to peptide analysis. For example, other analytes that can be detected using a system of the invention include (non-proteinaceous) biomarkers, antibiotics or other drugs, DNA, metabolites and small biological and non-biological molecules. Exemplary analytes include various sub-classes of small-molecule biomarkers, such as steroids, carbohydrates, amino acids, nucleotides, hormones, fatty acids, vitamins, flavins, protein-cofactors, lipids, phenolic compounds. In one embodiment, the analyte of interest is a biopolymer, preferably selected from the group consisting of a protein, a polypeptide and an oligopeptide. In one aspect, the analyte is a substance having a mass in the range of between 200 and 5000 Da, for example in the 200-500 Da range or in the 500 and 1700 Da range See in particular FIG. 22, demonstrating capture and detection of distinct small molecules (flavins, vitamins) of non-proteinaceous nature by an aromatically modified pore-forming toxin of the invention. Vitamin B12, also known as cyanocobalamin, is a non-protein molecule with a molecular weight of 1355 Da.

The invention also provides a method for providing a system according to the invention. Typically, this comprises the steps of:
providing recombinant monomers of said mutant pore-forming toxin or pore-forming fragment thereof;
contacting said monomers with liposomes and/or surfactants to assemble them into oligomers;
recovering the oligomers from the liposomes and/or surfactants; and
contacting the oligomers with a membrane, which may contain sphingomyelin, to allow the formation of nanopores.

Further embodiments of the invention relate to means and methods for preparing mutant protein pores by recombinant technology. These include a nucleic acid molecule encoding a mutant nanopore according to the invention, and an expression vector comprising said nucleic acid molecule. Also encompassed is a host cell, preferably a bacterial host cell, comprising the nucleic acid-containing expression vector.

An analytical system of the invention may be incorporated, e.g. in the form of an array of multiple systems, into a device. The device may be any conventional device for analyte analysis, such as an array or a chip. Provided is a device comprising a plurality of analytical systems (sensors) according to the invention. The plurality of sensors can be based on the same proteinaceous nanopore (i.e the same mutant), or on distinct nanopores inserted into a plurality of membranes, which may for example be connected to a plurality of electrical circuits to address and measure each nanopore sensor separately. Preferably, a single pore is present in each membrane. The pores might differ by their type, family, mutation etc. In one embodiment, a device comprises multiple pores to generate different characteristic signals from peptides, which signal can be compared or combined to improve their discrimination and characterization.

The protein nanopore of the invention may be present in a membrane, or inserted when required. Protein nanopores are typically asymmetric, and may be inserted with directional control relative to the cis and trans compartments of an analytical system by various means known in the art. Typically, and unless stated otherwise in the examples herein, nanopores are inserted from the cis compartment.

Any membrane may be used in accordance with the invention. Suitable membranes are well-known in the art. The membrane is preferably an amphiphilic layer. An amphiphilic layer is a layer formed from amphiphilic molecules, such as phospholipids, which have both hydrophilic and hydrophobic/lipophilic properties. The amphiphilic layer may be a monolayer or a bilayer. The membrane is preferably formed from a bilayer of phospholipids. The membrane is preferably formed from lipids or amphipathic molecules that are chemically stable under low pH conditions, for example ether-linked phospholipids. The amphiphilic molecules may be synthetic or naturally occurring. Non-natural amphiphiles that form a monolayer are known in the art and include, for example, block copolymers (di-block, tri-block, tetra-block etc) of various polymeric compositions.

### Analytical Methods

An analytical system or device finds its use in a variety of analytical methods. For example, it is advantageously used in single molecule analysis. A further embodiment of the invention therefore relates to a method for single molecule analysis, the method comprising adding a substance or mixture of substances to be analyzed to the chamber of a (plurality of) analytical system(s) as provided herein, allowing the substance(s) to contact the (lumen-facing region of the) nanopore, and detecting/characterizing at least one property of the substance (also referred herein as “analyte” or “analyte of interest”). The substance/analyte can be detected by a change in the electrical current through the nanopore. For example, various properties of the substance, e.g. including, but not limited to, volume, shape, charge, structure, cross-linking, post-translational modifications (phosphorylation, glycosylation, rhamnosylation, etc.), damage, (D/L-) chirality, and sequence can be detected. Preferably, the method comprises the identification and/or sequencing of a substance. The analytical system or method is surprisingly suitable for the analysis of an analyte having a mass in the range of between 200 and 5000 Da, for example 500-1700 Da.

The substance, for example a peptide analyte, is typically present in any suitable sample. The invention is typically carried out on a sample that is known to contain or suspected to contain the analyte. Alternatively, the invention may be carried out on a sample to confirm the identity of the analyte whose presence in the sample is known or expected. The sample may be a biological sample. The invention may be carried out in vitro using a sample obtained from or extracted from any organism or microorganism (e.g. archaeal, prokaryotic or eukaryotic). The sample is preferably a fluid sample. The sample may comprises a body fluid of a patient (e.g. urine, lymph, mucus or amniotic fluid, or preferably sweat, saliva blood, plasma or serum). The sample may be human in origin, but alternatively it may be from another animal, such as from commercially farmed animals, or of plant origin. The sample may be a non-biological sample. Examples of non-biological samples include surgical fluids, water such as drinking water, sea water or river water, and reagents for laboratory tests.

The sample may be processed (pretreated) prior to being used in the invention, for example by various purification means known in the art to isolate mixtures of proteins/peptides/molecules or target proteins/peptides/molecules. These may include for example affinity binding methods, such as antibodies, or chromatographic methods, to isolate and purify specific components of the sample or remove unwanted background impurities. For protein samples the proteins contained therein are preferably fragmented into peptides (preferably defined populations), for example by enzymatic means known in the art (e.g. proteases) or other degradative means. An analytical method of the invention may include one or more sample preparation steps. For example, a pre-filtering step and/or other modifications as done for other methods (e.g. Mass Spec).

A person of skill in the art would understand that many of the sample preparation methods employed for classical mass spectrometry can be used herein. For example, proteins in a sample might be denatured by physical (e.g. temperature) or chemical (e.g. chaotropic agents, detergents) means prior to processing and nanopore sensing. Alternatively, cross-links such as disulphide bridges can be broken to disrupt certain secondary structures. Alternatively, modifications, for example large glycans, might be modified, truncated or removed prior to nanopore sensing. Alternatively, some of the amino-acids in a peptide sample might be modified to alter the signal, such as for example, Cysteines or Lysines might be chemically labelled with additional tags to modulate the signal in nanopore sensing to provide further insight into the analyte. In some embodiments of the invention the peptide analytes might be subjected to reactions that alter the N-terminal or C-terminal ends of the molecules using methods known in the art, for example for the purposes of adding a molecular label or tag (e.g. to add a barcode to register the precursor sample, or to facilitate capture and detection in a nanopore system). Such molecular labels/tags might be peptide based, polynucleotide based or composed of other chemistries.

In one aspect, the invention provides a method for single molecule analysis, the method comprising adding a substance or mixture of substances to be analyzed to the chamber of an analytical system as provided herein, allowing the substance(s) to contact the (lumen-facing region of the) nanopore, and detecting/characterizing at least one property of the substance, wherein the substance is a proteinaceous substance, preferably a peptide, more preferably a peptide up to about 30, 20, 15, 10, 5, 3 or 2 amino acids in length.

The method may involve detecting a mutation and/or post-translational modification of a substance, for example detecting peptide fragments that differ by a single amino acid residue, degree of phosphorylation and/or degree of glycosylation. In specific embodiments, the nanopore detects peptides resulting from a protein mixture that has been subjected to denaturing conditions, or from a protein mixture that has been subjected to fragmenting conditions, including protease digestion e.g. as typically used in MS analysis. In one aspect, the fragmentation condition leads to positively charged peptide fragments. A method of the invention has the ability to quantify the absolute or relative abundance of the proteins in the original mixture from the peptide spectrum.

In certain embodiments of the invention, optimal conditions for peptide detection are performed under low pH conditions, preferably below pH 4.5, preferably below pH 4.0. Under physiological pH conditions, naturally occurring peptides have a wide range of charge distributions and net charges (e.g. both net positive and net negative) as a result of their highly variable composition of acidic, basic and neutral amino acids. This diversity of charge significantly complicates the ability to capture and detect all the peptides in a diverse mixture of different peptides in a nanopore sensing system when a fixed applied potential is applied, since not all peptides will experience the same net electrophoretic force. Depending on the polarity of the applied potential and the specific charge composition of each peptide, some peptides will experience net electrophoretic force into the nanopore, while other oppositely charged peptides will experience net electrophoretic force out of the nanopore. By implementing low pH conditions on the side of the nanopore sensing system containing a complex peptide analyte mixture, and preferably on both the cis and trans sides of the membrane, the amino acids of the peptide analytes become protonated. The increased protonation serves to both 1) increase the net positive charge of all peptides in a diverse mixture, and 2) create a more uniform distribution of charges in the peptide mixture. The increased net positive charge allows for an improved electrophoretic capture of the peptides in a nanopore system held under an appropriate polarity applied potential (e.g. when a negative potential is applied to the electrode on the opposite side of the membrane to the peptide analytes).

An improved uniformity of charge in the peptide mixture is also highly advantageous as all peptide molecules will experience more similar electrophoretic forces acting upon them under an applied potential. Since electrophoresis is an important component determining the efficiency of analyte capture into a nanopore, this reduces capture efficiency biases between different peptide compositions in mixtures. This highly advantageous feature reduces the likelihood that some peptide populations with inefficient capture will be missed or lost in the background of peptide populations with higher capture efficiency.

Implementing low pH conditions also alters the charge characteristics of the nanopore in the sensing system by partially protonating some of the water-facing amino acids. Notably, the increased positive charge inside the nanopore channel, and inside the lumen recognition region, alters the capture and subsequent detection of peptide analytes. Under low pH conditions the increased positive charge in the nanopore can electrostatically repel the mostly positively charged peptide analytes, which can in turn reduce capture efficiency and/or reduce the residence time of peptides inside the nanopore. This can reduce the ability to detect and characterize some peptide analytes.

A variety of different types of measurements may be made on the nanopore system. This includes without limitation: electrical measurements and optical measurements. Possible electrical measurements include: current measurements, impedance measurements, tunnelling measurements, and field-effect-transistor (FET) measurements of local voltage changes. Optical and electrical measurements may be combined to provide additional information (Heron et al. J Am Chem Soc 2009). Optical measurements may be employ dye systems that are reporters of ionic flux (Heron et al. J Am Chem Soc 2009).

The method is preferably carried out with a potential applied across the membrane. The applied potential may be a voltage potential. The applied voltage enables electrophoretic and/or electroosmotic flow through the nanopore to facilitate analyte capture and detection. Following convention, unless defined otherwise for the data in the invention herein, the active electrode is defined as that in the trans compartment, and is the one at which the stated polarity of potential is applied (e.g. relative to the ground electrode in the cis compartment). A person of skill in the art will understand that alternative electrode configurations are known in the art, and can be employed for example to control electrophoretic and/or electroosmotic analyte capture in the nanopore system through application of an applied potential, and/or to measure changes in ionic current or local voltage. The applied potential might be held at a constant voltage for a fixed period (milliseconds, seconds, minutes, hours). Alternatively, the voltage might be changed in discreet steps to alter the sensing conditions and/or obtain different information from the analytes. The voltage might be constantly changing, for example a person skilled in the art would understand that various pattern (e.g. square wave, triangular wave, sinusoidal, etc) waveforms might be employed to control analyte capture and obtain different characteristics from the analytes. Alternatively, the applied potential may be a chemical potential (e.g. a salt gradient across a membrane). The voltage used is typically from +50 V to −50 V, or +100 V to −100 V. The voltage used is preferably in a range having a lower limit selected from −300 mV, −300 mV, −150 mV, −100 mV, −50 mV, −20 mV and 0 mV and an upper limit independently selected from +10 mV, +20 mV, +50 mV, +100 mV, +150 mV, +200 mV, +300 mV. The voltage used is more preferably in the range +−50 mV to +−150 mV and most preferably in the range of +−50 mV to +−100 mV. The method is typically carried out with any well-known charge carriers present in the aqueous solution in the chamber, such as metal salts or ionic liquids. For example alkali metal salt, halide salts, for example chloride salts, such as alkali metal chloride salt, or ionic liquids or organic salts such as tetramethyl ammonium chloride, trimethylphenyl ammonium chloride, phenyltrimethyl ammonium chloride, or 1-ethyl-3-methyl imidazolium chloride. The salt is preferably potassium chloride (KCl), sodium chloride (NaCl), or lithium chloride (LiCl). The solutions may also contain well-known redox salts to mediate electron transfer at suitable electrodes, for example potassium ferrocyanide and potassium ferricyanide or other well-known redox couples. The salt concentration may range from 0.1 to 3 M, or up to the saturation point for a given salt type. The salt concentration is preferably from 0.1 to 1.5 M, and most preferably 0.15 to 1.0 M. The method is typically carried out in the presence of a buffer. In the exemplary apparatus discussed above, the buffer is present in the aqueous solution in the chamber. Any buffer may be used in the method of the invention. Typically, the buffer is bis-tris buffer, citrate buffer, phosphate buffer, HEPES buffer or Tris-HCl buffer. The methods are typically carried out at a pH below 8.0, and preferably at below pH 4.5, most preferably below pH 4.0, using buffers that are appropriate to this range (e.g. citrate buffer). The method may be carried out at from 0° C. to 100° C., preferably from about 20° C. to about 400 C.

Electrical measurements may be made using standard single channel recording equipment such as that described herein. Alternatively, electrical measurements may be made using a multi-channel systems known in the art that are capable of simultaneously acquiring signals from multiple independent nanopore systems (e.g. a plurality of membranes containing inserted nanopores).

The method of the invention may involve measuring multiple characteristics of the current signal, most preferably of event blockades arising from capture and detection of analytes. The one or more characteristics are preferably selected from: the open-pore current, the average or median current of the event blockade, the duration (dwell) time of the event blockade, the frequency of event blockades, the number of event blockades, the noise in the event blockade, and the shape of the event blockade (including stepwise changes). A person of skill in the art would understand that a range of analytical tools can be used to extract high level information from event blockades and other parts of the current signals. For example, edge-detecting algorithms can be used to segment the event blockades to simplify the data. Alternatively, the raw data may be analyzed directly, with or without the application of filters, for example using sliding window features and algorithms with long range memory, to extract characteristic metrics.

The method of the invention may involve determining one, two, three, four or five or more characteristics of the analyte from the characteristic metrics of the signals. The one or more characteristics are preferably selected from: the length of the analyte, the volume of the analyte, the mass of the analyte, the shape of the analyte, the charge distribution of the analyte, the identity of the analyte, the sequence of the analyte, any chemical modifications of the analyte. The characteristics of the analytes can be determined by any number of a wide range of analytical methods known in the art, including for example statistical methods or machine learning methods. These methods may have been trained or optimized by training the systems with model analytes for example, or may have been built from first principles. For example, the identity of peptides can be determined by comparison to previously acquired data using training data. Also provided herein is an analytical method of determining the identity of the original protein/s from the peptide fingerprint by comparing the spectrum to theoretical data or previously trained data. A person of skill in the art will understand that the multi-metric data obtained for each nanopore event can be exploited in higher dimension analysis (e.g. by combined comparison of 2, 3, 4, 5, 6, or more separate event metrics) to discriminate different analytes that might not be separable by any one metric alone. A person of skill in the art will also appreciate that a collection (spectra) of multiple analyte events can be analyzed as population ensembles for the discreet populations of analytes in a sample, and that the discreet populations might be resolved (e.g. in multiple dimensions using multiple metrics as axes) and identified using any number of advanced fitting and classification tools. Further, unique data from the populations, for example fingerprints, might be used in analytical methods to identify the analyte composition, and therefore for example for digested peptide mixtures identify and/or quantify the precursor protein(s).

As is exemplified herein below, the present inventors found that for certain protein nanopores, such as Aerolysin and CytK, it is either essential or highly advantageous to reduce the net positive charge in the nanopore channel, preferably in the recognition region, most preferably at or near the constriction, preferably in combination with aromatic mutations, to enable efficient capture and recognition of peptide analytes under low pH conditions. For example, by introducing acidic residue(s) (Asp/Glu) by substitution adjacent to the aromatic mutation(s) (Phe/Tyr/Trp). Hence, in one embodiment the pore comprises one or more mutation(s) to Glu and/or Asp residue(s) in the water-facing region. Alternatively, it is understood that net positive charge can be also reduced by replacing basic residue(s) (Arg/Lys/His) with neutral or acidic residue(s), optionally by substitution with aromatic residues that also separately and additively improve peptide capture and discrimination (e.g. CytK-K128F, Aer-K238F examples contained herein).

Increased positive charge in the channel of the nanopore under low pH conditions also alters the ion selectivity of the nanopore. The increased positive charge in the nanopore channel favors increased transport of anionic species and decreases the transport of cationic species, which in turn alters the net electro-osmotic flux of hydrated ions flowing through the nanopore under an applied potential. The increased anionic electro-osmotic flux through the nanopore will act against the electro-phoretic forces acting on the mostly positively charged peptide analytes under low pH conditions.

The direction and magnitude of the electro-osmotic component for a nanopore system can be determined by ion-selectivity measurements known in the art. For example, nanopore ion-selectivity can be measured in an in vitro electrophysiology system by measuring the reversal potential under asymmetric salt conditions (e.g. with 2M KCl in the trans compartment 0.5 M KCl in the cis compartment). Table 3 herein below contains the measured reversal potentials and ion-selectivity for selected Aerolysin and CytK nanopores. FraC ion-selectivity under low pH has been determined previously (Huang et al. Nat. Commun. 2019).

For nanopore sensing systems set up to enable analyte capture by electrophoresis, (e.g. in a system with a negative potential applied at the electrode in the compartment opposite to that containing the peptide analytes), such as for the FraC nanopore and CytK nanopore examples contained herein, it is advantageous to ensure that excessive electro-osmotic forces do not act against electrophoretic capture of analytes under the chosen sensing conditions. Therefore, in certain embodiments of the invention (e.g. for the CytK nanopore examples contained herein) where ion-selectivity and electro-osmotic flux are increased by the implementation of low pH conditions, it can be advantageous to reduce the net ion-selectivity and electro-osmotic flux, preferably to a level where electrophoretic forces dominate analyte capture, preferably to close to zero net ion-selectivity. Electro-osmosis can be reduced by reducing the net charge inside the nanopore channel (e.g. by mutagenesis. See Table 4). For example, the anion ion-selectivity bias and resulting net anionic electro-osmotic flux can be reduced by introducing acidic residues by substitution adjacent to the aromatic mutations. Hence, in one embodiment the pore comprises one or more mutation(s) to Glu and/or Asp residue(s) in the water-facing region. Alternatively, it is understood that net positive charge can be also reduced by replacing basic residues with neutral or acidic residue(s), optionally by substitution with aromatic residue(s) that also separately and additively improve peptide capture and discrimination (e.g. CytK-K128F, Aer-K238F examples contained herein).

In some proteins, like FraC, FraE or FraB, the wild-type pores already contain sufficient negative charge characteristics inside the water facing nanopore channel/lumen or recognition region under low pH conditions for optimal ion-selectivity and electro-osmosis, and optimal interaction with mostly positively charged analytes, and therefore do not require mutations to add further negative charges in spatial combination with the aromatic residue(s) that are introduced. Conversely, removing acidic residues in the FraC example herein, which increases the net positivity of the nanopore, dramatically reduced peptide capture and discrimination under an electrophoretic dominant regime.

In some embodiments of the invention, it has been found that efficient peptide analyte capture and detection can achieved under conditions set up to create dominant electro-osmotic capture. For example, for the Aerolysin nanopore system examples herein, the implementation of low pH conditions increases the net positive charge inside the nanopore channel, resulting in increased anion selectivity, and a strong net anion-selective nanopore (see Table 3) and in increased electrostatic repulsion of mostly positively charged analytes. The resulting strong electro-osmotic flux through the nanopore can be exploited to capture analytes against the direction of the electrophoretic forces acting upon them (e.g. with a positive applied potential at the trans electrode for a system with mostly positively charged peptides in the cis solution—see Aerolysin examples herein). For certain embodiments of the invention, it can be highly advantageous to exploit electro-osmotic forces to capture analytes since it is less sensitive to charge composition. It can therefore be highly advantageous for capturing and detecting a diverse composition of unlabeled peptides (e.g. neutral, net positive, net negative). In certain embodiments of the invention, the strength of electro-osmotic force acting on the analytes can be further tuned (e.g. by mutagenesis). For example, in certain embodiments, it can be useful to reduce the electro-osmotic force to increase the duration for which the analytes are retained in the nanopore. For example, the anion ion-selectivity bias and resulting net anionic electro-osmotic flux that results from low pH conditions can be reduced by introducing acidic residues, preferably by substitution adjacent to the aromatic mutations. Acidic mutation substitutions that reduce net positive charge will also reduce electrostatic repulsion of mostly positively charge analytes. Hence, in one embodiment the pore comprises one or more mutation(s) to Glu and/or Asp residue(s) in the water-facing region. Alternatively, it is understood that net positive charge can also be reduced by replacing basic residues with neutral or acidic residues, optionally by substitution with aromatic residues that also separately and additively improve peptide capture and discrimination (e.g. CytK-K128F, Aer-K238F). It is also understood that mutagenesis can be combined with changes to the system conditions (e.g. pH, salt type, salt asymmetry) to control the direction and magnitude of the electro-osmotic effect, and can be determined experimentally as described previously by measurements of reverse voltages for example.

Hence, described herein are methods for optimizing the electrophoretic and electro-osmotic components of a nanopore sensing system for the capture and characterization of unlabeled peptides for the purpose of discriminating between different peptides. The optimal “characterization parameters” for effective peptide sensing or sensing of other molecules can be determined experimentally in a nanopore system by measurement using model peptides or natural peptides.

### Kit of Parts

The invention also provides a kit of parts, e.g. for use in characterizing an analyte of interest, the kit comprising (i) a mutant proteinaceous nanopore, an analytical system and/or a device according to the invention; and (ii) an analyte-handling enzyme. Preferably, the analyte-handling enzyme is a protein-handling enzyme, such as a protease. Of particular interest are trypsin or other proteases such as chymotrypsin or Lys-C protease. The use of trypsin can be advantageous because it cleaves preferentially after a K/R amino acid and as most peptides will have a positive charge next to the zwitterionic charges on the peptide, yielding an additional net charge of +1 under acidic to neutral pH conditions employed for analysis. Lys-C protease has high activity and specificity for lysine residues, resulting in larger peptides and less sample complexity than trypsin (i.e., fewer peptides). Unlike trypsin, Lys-C protease can cleave lysines followed by prolines, making it ideal for sequential protein digestion followed by trypsin to decrease missed cleavages. These unique Lys-C protease properties ensure high digestion efficiency when used alone or followed by tryptic digestion.

As will be appreciated by a person skilled in the art, an analytical system, device or a kit comprising a mutant proteinaceous pore as herein disclosed finds many uses and applications e.g. in the field of molecular analysis and identification. These include single molecule analysis, preferably the identification and/or sequencing of a biomolecule or biopolymer, more preferably label-free protein or peptide fingerprinting.

Further embodiments of the invention are as follows.

<1> A proteinaceous nanopore comprising a mutant beta-barrel protein pore-forming toxin, or a pore-forming fragment thereof, wherein the lumen-facing recognition region of the pore-forming protein or fragment thereof comprises one or more substitution(s) of lumen-facing non-aromatic amino acid(s) to a natural or non-natural aromatic amino acid residue.
<2> Proteinaceous nanopore according to <1>, comprising a mutant pore forming toxin comprising one or more substitution(s) of lumen-facing amino acid(s) to Trp, Tyr or Phe.
<3> Proteinaceous nanopore according to <1> or <2>, wherein the beta-barrel pore-forming toxin has an internal diameter (pore size; constriction) in the recognition region in the range of 0.2 to 2.0 nanometers, preferably a minimum internal diameter of 0.5 to 1.5 nanometers.
<4> Proteinaceous nanopore according to any one <1> to <3>, comprising a mutant pore selected from the group consisting of alpha-hemolysin (SwissProt P09616.2), aerolysin (SwissProt P09167), Gamma-hemolysin component B (SwissProt P0A075.1) lysenin (SwissProt 018423), epsilon-toxin (ETX), hemolytic lectin (LSL; SwissProt Q868M7), cytolysin k (cytK; SwissProt Q937V2), and functional homologs showing at least 80%, preferably at least 85%, more preferably at least 90% sequence identity therewith.
<5> Proteinaceous nanopore according to <4>, selected from the group consisting of aerolysin, lysenin and cytolysin k (cytK), and functional homologs showing at least 90%, preferably at least 95%, more preferably at least 98% sequence identity therewith.
<6> Proteinaceous nanopore according to any of <1> to <5>, comprising aerolysin (Aer) comprising an aromatic amino acid substitution in the water-facing region of the pore, which region runs from residues 212 to 242 and from 256 to 284 of aerolysin, preferably wherein the aromatic mutation is at least in the region 212-242, more preferably wherein the mutant is either W, Y or F substituted at one of more positions including Q212, G214, D216, T218, R220, D222, A224, N226, S228, T230, T232, G234, S236, K238, T240 or K242.
<7> Proteinaceous nanopore according to any one of <1-5>, comprising a mutant CytK, preferably comprising an aromatic amino acid substitution in the lumen-facing region of the pore, which region runs from residue 112 to residue 155, more preferably one or more (e.g. up to 5, preferably up to 4, more preferably up to 3 or 2) of the following lumen-facing non-aromatic transmembrane residues E112/T114/T116/S118/S120/Q122/G124/S126/K128/S130/T132/S134/S137/E139/G141/T 143/Q145/T147/S149/S151/S153/K155, such as one or more of E112/T114/T116/S118/S120/Q122/G124/S126.
<8> Proteinaceous nanopore according to any one of <1-5>, comprising mutant Lysenin (UniProtKB—P13423) or a pore-forming fragment thereof, preferably comprising an aromatic substitution at position 35, 37, 39, 41, 45, 43, 47, 49, 51, 53, 55, 57, 59, 63, 61, 65, 68, 74, 76, 78, 80, 82, 84, 86, 88, 90, 92, 94, 96, 98, 100, 102 and/or 104, more preferably wherein the Lys mutant comprises an aromatic substitution at position Glu76, for example mutant Lys-E76F.
<9> Proteinaceous nanopore according to any one of <1-8>, wherein one or more further mutation(s) is/are introduced in the lumen-facing amino acids of the recognition region, which further mutation(s) increase the net negative charge of the pore.
<10> Proteinaceous nanopore according to <9>, comprising one or more mutation(s) to Glu and/or Asp residue(s).
<11> Proteinaceous nanopore according to any one of <1-10>, comprising a mutant beta-barrel pore or fragment thereof selected from the group consisting of:
(i) Aer-K238D, Aer-K238D-A260F, Aer-K238D-S264F, Aer-K238D-Q268F and Aer-K238D-S272F;
(ii) CytK-Ser126Tyr, CytK-Ser126Trp, CytK-Ser126Phe, (ii) CytK-Lys128Tyr, CytK-Lys128Trp, CytK-Lys128Phe, S120W/F/Y+K128D, Q122W/F/Y+K128D, G124W/F/Y+K128D, S126W/F/Y+K128D; and
(iii) Lys-E76F.
   <12> An analytical system comprising a hydrophobic membrane separating a fluid chamber into a cis side and a trans side, said membrane comprising a mutant proteinaceous nanopore according to any one of <1-11>.
   <13> A method for providing a system according to <12>, comprising the steps of
providing recombinant monomers of said mutant pore-forming toxin or pore-forming fragment thereof;
contacting said monomers with liposomes and/or surfactants to assemble them into oligomers;
recovering the oligomers from the liposomes and/or surfactants; and
contacting the oligomers with a membrane, which may contain sphingomyelin, to allow the formation of nanopores.
   <14> A method for single molecule analysis, preferably for identification and/or sequencing of an analyte of interest comprising adding an analyte of interest to a chamber of an analytical system according to <12>, allowing the analyte to contact the nanopore, and detecting/characterizing at least one property of the analyte.
   <15> Method according to <14>, comprising subjecting the nanopore to an electric field such that the analyte is electrophoretically and/or electroosmotically captured in the nanopore.
   <16> Method according to <14> or <15>, wherein the analyte of interest has a mass in the range of between 200 and 5000 Da, preferably in the range between 200 to 500 Da or 500 to 1700 Da.
   <17> Method according to any one of <14-16>, wherein the analyte of interest is a biopolymer, preferably selected from the group consisting of a protein, a polypeptide and an oligopeptide.
   <18> Method according to <17>, wherein the analyte of interest is a proteinaceous substance, preferably a peptide, more preferably a peptide up to about 30, 20, 15, 10, 5, 3 or 2 amino acids in length.
   <19> Method according to <17> or <18>, comprising detecting a mutation and/or post-translational modification of an analyte, for example detecting peptide fragments that differ by a single amino acid residue, amino acid chirality, degree of phosphorylation and/or degree of glycosylation.
   <20>. Method according to any one of <14-19>, wherein detection is performed at a pH≤4.5, preferably below pH 4.0.
   <21> A method of decreasing translocation speed of a peptide analyte through a transmembrane beta-barrel protein pore, comprising:
(a) increasing the net aromaticity of the lumen of the pore by substituting one or more lumen-facing non-aromatic amino acid(s) with one or more aromatic amino acid(s); and
(b) passing the polypeptide through the pore, wherein increasing the net aromaticity decreases the translocation speed of the polypeptide through the pore.
   <22> Method according to <21>, wherein step (a) comprises providing a proteinaceous nanopore according to any one of <1-11>.
   <23> A device comprising a plurality of analytical systems according to <12>, preferably wherein the analytical systems comprise distinct pore types.
   <24> A kit of parts, for characterizing an analyte of interest comprising (i) a mutant proteinaceous nanopore according to any one of <1-11>, an analytical system according to <12>, or a device according to <23>; and (ii) an analyte-handling enzyme, preferably a protease.
   <25> The use of an analytical system according to <12>, a device according to <23> or a kit according to <24>, for single molecule analysis, preferably for identification and/or sequencing of a biomolecule or biopolymer, more preferably for label-free protein fingerprinting.

### LEGEND TO THE FIGURES

FIG. 1. Actinoporins common sequence alignment and wild-type Fragaceatoxin C. A: Common sequence alignment of some known actinoporins, the dots represent the same amino acid as the common sequence, other amino acid differences between the pores are represented by their single-letter code. Figure discloses SEQ ID NOS 52-68, respectively, in order of appearance. B: Artistic model of Fragaceatoxin C (PDB: 4TSY) inserted into a lipid bilayer, across which a voltage is applied. Several non-conserved positions are enlarged. C: Representative traces of the octameric (T1) and heptameric (T2) form of wild-type Fragaceatoxin C under an applied potential of −50 mV in 1M KCl and 50 mM citric acid titrated with bis-tris propane to pH 3.8. Traces were collected at a sampling frequency of 50 kHz, using a 10 kHz Bessel filter and 5 kHz Gaussian filter.

FIG. 2. Alignment between Fragaceatoxin C homologues. Positions in homologs corresponding to D10 and G13 in Fragaceatoxin C are highlighted. Figure discloses SEQ ID NOS 69-87, respectively, in order of appearance.

FIG. 3. Electrophysiology recordings of (mutant) Fragaceatoxin C with trypsin digested lysozyme. A: Representative electrical ionic current traces of (mutant) Fragaceatoxin C combined with equal units of trypsin digested lysozyme added to the cis side and under an applied potential of −50 mV. The current traces show representative sections of ionic current data for various pores. The lowest current level is the open-pore current of the pore (I_{O}), and the step-like upwards events are the result of captured analytes occluding a portion of the ionic current flowing through the nanopore (event blockades, I_{B}). B-D: representative trace of octameric Fragaceatoxin C (T1, B), heptameric Fragaceatoxin C (T2, C), and Fragaceatoxin C mutant G13F (D). The raw current data in the traces are overlaid with a fit line from the application of edge-detecting event detection algorithms. The block above the trace aligns with the length of the events to indicate the duration of the pulses. Traces were collected in 1M KCl and 50 mM citric acid titrated with bis-tris propane to pH 3.8 at a sampling frequency of 50 kHz, using a 10 kHz Bessel filter and 5 kHz Gaussian filter.

FIG. 4. Event count and signal correlation of (mutant)’ Fragaceatoxin C with trypsin digested lysozyme. A-D: Observed excluded current (Iex %) spectra from tryptic digest of lysozyme. A: octameric wild-type Fragaceatoxin C (T1), B: heptameric wild-type Fragaceatoxin C (T2), C: Fragaceatoxin C mutant G13F and D: Fragaceatoxin C mutant G13N. Traces were collected at −50 mV in 1M KCl and 50 mM citric acid titrated with bis-tris propane to about pH 3.8 at a sampling frequency of 50 kHz, using a 10 kHz Bessel filter and 5 kHz Gaussian filter. E: Squared first derivative Euclidean cosine correlation of residual current spectra of (mutant) Fragaceatoxin C combined with equal units of trypsin digested lysozyme. The black boxes surrounding multiple mutants represent similar signals. Traces were collected in 1M KCl and 50 mM citric acid titrated with bis-tris propane to pH 3.8 at a sampling frequency of 50 kHz, using a 10 kHz Bessel filter and 5 kHz Gaussian filter. The external bias was −50 mV except for D10R # and G13H #, which were tested at +50 mV.

FIG. 5. Peptide recognition of (mutant) Fragaceatoxin C. A: (A) location of mutations in the lumen of Fragaceatoxin C (modeled on PDB: 4TSY) are marked by arrows. B: Gaussian fits to histograms of the excluded currents from the clustered event blockade for the capture and detection of Angiotensin IV [1], Angiotensin III [2], Angiotensin I [3] and Angiotensinogen [4] recorded under an applied potential of −50 mV. C: excluded current % (I_{EX}%) versus dwell time scatter plots of the single-molecule peptide event blockades detected by the different pore types. Traces were collected in 1M KCl and 50 mM citric acid titrated with bis-Tris propane to pH 3.8 at a sampling frequency of 50 kHz, using a 10 kHz Bessel filter and 5 kHz Gaussian filter.

FIG. 6. Peptide recognition of (mutant) Fragaceatoxin C. Peptide recognition in further pore types, including heptameric and hexameric Fragaceatoxin C. (Top panel) The fit of the residual current is shown for Leucine-enkephalin (YGGFL (SEQ ID NO: 36)) [Leu-enk], Angiotensin II (4-8) (YIHPF (SEQ ID NO: 37)) [AngII] and Kemptide (LRRASLG (SEQ ID NO: 38)) [kemptide] each in 10 μM concentration, recorded under an applied potential of −70 mV. (Bottom panel) Excluded current % (I_{EX}%) versus dwell time scatter plots of the single-molecule peptide event blockades for the different pore types. Traces were collected in 1 M KCl and 50 mM citric acid titrated with bis-Tris propane to pH 3.8 at a sampling frequency of 50 kHz, using a 10 kHz Bessel filter and 5 kHz Gaussian filter. The figure shows that aromatic nanopores can identify and discriminate between different peptides better than the wildtype Fragaceatoxin C.

FIG. 7. Electrophysiology setup of an analytical system comprising a nanopore. The schematic shows an example of one type of system that can be used with nanopore sensors for the electrical detection of analytes. Other types of systems are also suitable, such as arrays of nanopore sensors on microchips for example. The schematic shows a chamber consisting of two compartments made of Delrin, separated by a Teflon film containing a 100 μm hole. Both compartments were filled with buffer and an electrode (eg. Ag/AgCl electrode) is connected to each chamber to facilitate electrical detection. A lipid membrane is formed over the hole inside the Teflon film using the Langmuir-Blodgett method to separate the two compartments. Nanopores are typically added from the cis chamber and allowed to insert into the membrane. Analytes are typically added to the cis chamber for detection.

FIG. 8. Concept of bottom-up nanopore-based proteomics. A: Artistic representation of protease protein digestion to digest a protein into a mixture of peptide fragments. Figure discloses SEQ ID NOS 88-89, respectively, in order of appearance. B: Artistic representation of the experimental setup where a peptide fragment from the resulting peptide fragment mixture is captured and translocated through a FraC nanopore by applying an electric field across the membrane. Figure discloses SEQ ID NO: 90. C: Artistic representation of the resulting ionic current data for detected peptides from a nanopore-based electrophysiology experiment. D: Artistic representation of a resulting residual current versus standard deviation spectrum obtained from analysis of the individual single-molecule event blockades, displaying distinct clusters for the different peptide populations.

FIG. 9. Excluded current—mass calibration using peptides and the spectrum obtained from tryptic lysozyme peptides. Characterization of G13F-FraC-T1 nanopores using synthetic model peptides that are predicted to result from the complete tryptic digestion Gallus-gallus lysozyme. A: Mass of the synthetic model peptides (circles) plotted against the average measured excluded current (%) for each peptide when added to the G13F-FraC-T1 nanopore system (obtained from n>3 multiple separate experiments on separate pores for each model peptide). The dashed line represents a logistic function fit through the data and shows a clear correlation between excluded current and molecular weight, which can be used to characterize captured peptides and for predictive purposes when testing unknown peptides. B. Excluded current spectrum (histogram of the excluded currents from event blockades) recorded from addition of a mixture of all the model peptides to a G13F-FraC-T1 pore. The peaks are labelled according to the predictions determined from the experiments in part A, and match the same position observed in the separate experiments. Figure discloses SEQ ID NOS 91-97, respectively, in order of appearance.

FIG. 10. Nanopore experiments compared to electrospray ionisation mass spectrometry. A. Residual current spectrum as obtained by nanopore electrophysiology using G13F-FraC-T1 and a tryptic digest of Gallus-gallus lysozyme. B. Mass spectrometry results from the same tryptic digest as A, but measured with a mass spectrometer (ESI-MS). The resulting peptide masses are mapped to residual current using the logistic function prediction shown in FIG. 9A with a standard deviation of 0.5 Iex %.

FIG. 11. Reproducibility of nanopore protein spectra. Each row presents three independent repeats of the sensing of proteolytic digestions of BSA (A), DHFR (B) and EFP (C) proteins. Each repeat was acquired from a separate nanopore experiment with a fresh nanopore, using the same digested sample in each repeat. The left-side panels show the excluded current histograms with a normalized area of 100%, which are obtained from the excluded current versus dwell time scatters of all event blockades shown in the respective right-side panels. All measurements were performed using G13F-FraC-T1 nanopores in 1M KCl buffered to pH 3.8 using 50 mM citric acid titrated with bis-tris-propane under an applied potential of −70 mV. Recording was performed at 50 kHz using an analog Bessel-filter at 10 kHz and a digital Gaussian filter of 5 kHz.

FIG. 12. Spectral matching using squared first difference correlation coefficient. A. Example representative baseline corrected residual current spectra of the measurement of peptide fragment mixtures from 9 tryptic digested proteins, shows that unique spectra are observed for each protein type. The right-side panels show the excluded current histograms with a normalized area of 100%, which are obtained from the excluded current versus dwell time scatters of all event blockades shown in the respective left-side panels. B. Leave-one-out spectral matching of the baseline corrected residual current spectra using Euclidean cosine cross-correlation.

FIG. 13. Detection of phosphorylated proteins. 2.5 μM of kemptide (LRRASLG (SEQ ID NO: 38)) and 2.5 μM of phosphorylated kemptide (LRRA{pS}LG (SEQ ID NO: 39)) were added to the cis-chamber of a system comprising FraC_G13F nanopores. Measurement in 1M KCl, 50 mM citric acid buffered with bis-tris propane to pH 3.8 Recordings were done at an applied potential of −70 mV at 50 kHz frequency with a 10 kHz lowpass filter. The graph shows that the peptides can be detected as two distinct clusters, plotting residual current (Ires=blockade current/open-pore current) versus dwell time.

FIG. 14. Detection of glycopeptides. 2.5 μM of unmodified peptide (ANVTLNTAG (SEQ ID NO: 40)), 2.5 μM of peptide with one glycan (ANVT(Glc)LNTAG (SEQ ID NO: 41) and 2.5 μM of peptide with two glycans (ANVT(Glc)LNTT(Glc)G (SEQ ID NO: 42)) were added sequentially to the cis-chamber of a system comprising FraC_G13F-T1 nanopores (3M LiCi, 50 mM citric acid buffered with bis-tris propane to pH 3.8, −50 mV at 50 kHz frequency with a 10 kHz lowpass filter). The figure shows the residual current blockade histogram from all detected capture events when measuring a mixture containing all three glycosylated peptides.

FIG. 15. Detection of rhamnosylated proteins. 25 μg of unmodified Elongation Factor P (EF-P, A) and 75 μg of rhamnosylated EF-P (B) were digested into peptide fragments using Lys-C. After digestion, in separate experiments 8 μg of digested protein was added to the cis-chamber of nanopore sensing systems comprising a FraC_G13F-T1 nanopore for peptide analysis (3M LiCl, 50 mM citric acid at pH 3.8, −50 mV at 50 kHz frequency with a 10 kHz lowpass filter). The rhamnosylation modification is on the SGRNAAVVK (SEQ ID NO: 43) peptide fragment. The rhamnosylation modification is clearly discriminated by the large shift in the residual current (Ires) between the modified peptide [SGR{rham}NAAVVK (SEQ ID NO: 44)] and the unmodified peptide [SGRNAAVVK (SEQ ID NO: 43)]. Figure discloses “NLLTGAGTETVFK” as SEQ ID NO: 98.

FIG. 16. Discrimination between single amino changes. (panel A) Detection of two forms of enkephalin with sequences added to the cis-chamber of G13F-FraC-T1 pores: YGGFL (SEQ ID NO: 36), and YdAGFdL (SEQ ID NO: 100), wherein d represents a D-amino acid; all other amino acids are L-. Measurements were performed in 1 M KCl, 50 mM citric acid titrated with bis-tris-propane (pH 3.8) at −100 mV applied potential sampling at 50 kHz and filtered to 10 kHz using the G13F-FraC-T1 pore. The figure plots the amplitude of the blockade versus the standard deviation of the noise in the blockade for the recorded event blockades, and illustrates that differences of at least 4 Da can be differentiated as two clear clusters. (Panel B and C) Difference in nanopore signal due to the presence of D-amino acids. A mixture 10 μM of [Ala2]-Leu Enkephalin and 10 μM DADLE ([D-Ala2, D-Leu5]-Enkephalin) was added to the cis compartment (FraC-G13F; panel B) or trans compartment (CytK-K128F; panel C). Measurement were performed in 3M LiCl, 50 mM citric acid, buffered to pH 3.8. Data recorded with 50 kHz sampling frequency and 10 kHz filter. FIGS. 16B-16C disclose “YAGFL” as SEQ ID NO: 99, and “YaGFl” as SEQ ID NO: 100.

FIG. 17. Detection of trypsinated lysozyme in Aerolysin nanopores. Representative electrical ionic current traces from (mutant) Aerolysin nanopores with 4 μg of trypsinated lysozyme added to the cis-chamber of a nanopore sensing system (+150 mV). The current traces show representative sections of ionic current data for selected pores, including WT-Aer at pH 7.5 (A), WT-Aer at pH 3.8 (B), Aer-K238F at pH 3.8 (C) and Aer-K238D-S264F at pH 3.0 (D). The open-pore current (I_{O}) and exemplary step-like current blockades (Is) from peptide captures are marked. Traces were acquired with 1M KCl in cis and trans, with 50 mM citric acid buffered with bis-tris propane to about pH 3.8 or pH 3.0, or with 50 mM Tris buffered at pH 7.5, as indicated.

FIG. 18. Detection of trypsinated lysozyme in Aerolysin nanopores. Structure or schematic of the aerolysin nanopore, with indicated locations of, and spacing between, the modifications, and residual current versus dwell time scatter of individual peptide blockades provoked by 4 μg of trypsinated lysozyme added to the cis-chamber of a nanopore sensing system comprising either WT-Aerolysin at pH 7.5 (A), WT-Aerolysin at pH 3.8 (B), K238F aerolysin at pH 3.8 (C), K238D aerolysin at pH 3.0 (D), K238D-A260F aerolysin at pH 3.0 (E), K238D-S264F aerolysin at pH 3.0 (F), K238D-Q268F aerolysin at pH 3.0 (G), K238D-S272F aerolysin at pH 3.0 (H). Measurements were performed in 1M KCl cis and trans, with 50 mM citric acid buffered with bis-tris propane to about pH 3.8 or pH 3.0 for low pH experiments, or with 50 mM Tris buffered at pH 7.5 as indicated. Recordings were done at an applied potential of +150 mV at 50 kHz frequency with a 10 kHz lowpass filter. The figure shows that aromatic mutations, especially in combination with modifications that increase the negative charge of the pore, improve the recognition of peptides especially at pH values lower than 4. (I) Measurement of 4 μg trypsinated lysozyme added to the cis compartment (final concentration 10 ng/μl) of nanopore system comprising Aer-K238W. Measurement in 1M KCl, 50 mM citric acid, buffered to pH 3.8 at +150 mV applied potential. Data recorded with 50 kHz sampling frequency and 10 kHz filter.

FIG. 19. Detection of trypsinated lysozyme in Cytolysin K (CytK) nanopores. Representative electrical ionic current traces from (mutant) Cytolysin K nanopores with 4 μg of trypsinated lysozyme added to the trans-chamber of a nanopore sensing system (+100 mV). The current traces show representative sections of ionic current data for selected pores, comprising either WT-CytK at pH 3.8 (A), CytK-K128F at pH 3.8 (B), or CytK-S126F-K128D at pH 3.8 (C). The open-pore current (I_{O}) and exemplary step-like current blockades (Is) from peptide captures are marked. Traces were acquired with 1M KCl in chambers and 50 mM citric acid buffered to pH 3.8.

FIG. 20. Detection of trypsinated lysozyme in Cytolysin K (CytK) nanopores. A: homology model of CytK (left) mapped onto the structure of the alpha-hemolysin nanopore from Staphylococcus aureus, and predicted beta-strand showing inward water-facing amino acids for the beta-barrel lumen of the nanopore (right). B-G: Residual current versus dwell time scatter of individual peptide blockades provoked by 4 μg of trypsinated lysozyme added to the trans-chamber of a system comprising either (B) wild type (WT-CytK) at pH 3.8, (C)K128F CytK nanopore at pH 3.8, (D) S126F-K128D CytK nanopore at pH 3.8, (E)S120F—K128D CytK nanopore at pH 3.0 (F) Q122F—K128D CytK nanopore at pH 3.0, (G) G124F—K128D CytK nanopore at pH 3.0. (H) Measurement of two peptides (10 μM Lys4 and 10 μM Lys7) added to the trans compartment a system comprising K128W CytK nanopore. Measurement in 1M KCl, 50 mM Tris, buffered to pH 7.5 at +100 mV applied potential. Data recorded with 50 kHz sampling frequency and 10 kHz filter.

To the left of each panel is indicated the schematic position of the substituted amino acid. Recordings were done at an applied potential of +100 mV at 50 kHz frequency with a 10 kHz lowpass filter. The figure shows that aromatic mutations especially in combination with modifications that increase the negative charge of the pore allow the recognition of peptides especially at pH values lower than 4.

FIG. 21. Detection of Lys-C digested lysozyme in Lysenin nanopores. Measurement of 0.5 μg Lys-C digested lysozyme added to the trans compartment (final concentration 1.25 ng/μl) of a system comprising either (A) wild type (WT-Lys) or (B) mutant Lys-E76F nanopores. Measurements were performed in 1M KCl, 50 mM Citric acid, buffered to pH 3.8 at −70 mV applied potential. Data were recorded with 50 kHz sampling frequency and 10 kHz filter.

FIG. 22. Detection of non-proteinaceous small molecules. Analytes were added to the cis-chamber (Thioflavin 2.0 μM) or to the cis and trans chambers (Vitamin B12, 10.0 μM) of a system comprising heptameric (A) wild-type FraC or (B, C) mutant FraC_G13F nanopores (Thioflavin); or octameric (D) wild-type FraC or (E, F) mutant FraC_G13F nanopores (Vitamin B12). Measurement in 1M KCl, 50 mM Tris.HCl pH 7.5 Recordings were done at an applied potential of −70 mV (Vitamin B12) or −50 mV (Thioflavin) at 50 kHz frequency with a 10 kHz lowpass filter. The graph shows that the molecules can be detected as a distinctive cluster, plotting residual current (Ires=blockade current/open-pore current) versus dwell time.

### EXPERIMENTAL SECTION

### Materials and Methods

Chemicals. Sphingomyelin (Porcine brain, >99%, CAS #383907-91-3) and diphytanoyl-sn-glycero-3-phosphocholine (DPhPC, >99%, CAS #207131-40-6) were retrieved from Avanti Polar Lipids. Ni-NTA resin was obtained from Qiagen. Lysozyme (Albumin free for tryptic digest, CAS #12650-88-3), Glucose (>99%, CAS #50-99-7), Sodium chloride (99.5%, CAS #7647-14-5), Potassium chloride (99%, CAS #7447-40-7), Dithiothreitol (DTT, >99.0%, 3483-12-3), Trizma® HCl (>99%, CAS #1185-53-1), Trizma® base (>99.9%, CAS #77-86-1), Imidazole (>99%, CAS #288-32-4), n-Dodecyl β-D-maltoside (DDM, >99%, CAS #69227-93-6), Hydrochloric acid (1 M, CAS #7647-01-0), Urea (>99.5%, CAS #57-13-6), Magnesium chloride (>98.5%, CAS #7786-30-3), LB Broth (Luria/Miller), Agar-agar and 2× YT Broth were obtained from Carl Roth. Ampicillin sodium salt (CAS #69-52-3), Isopropyl β-D-1-thiogalactopyranoside (IPTG, ≥99%, CAS #367-93-1), Ethanol (≥99.8%, CAS #64-17-5) and all enzymes were received from Fisher Scientific. Lysozyme from chicken egg white (for Lysis, CAS #12650-88-3), N,N-Dimethyldodecylamine N-oxide (LDAO, ≥99.0%, CAS #1643-20-5), Pentane (≥99%, CAS #109-66-0), Iodoacetamide (IAA, ≥99%, CAS #144-48-9), Bis-tris propane (≥99.0%, CAS #64431-96-5) were bought from Sigma-Aldrich. n-Hexadecane (99%, CAS #544-76-3) and Citric acid (99.6%, CAS #77-92-9) were purchased from Acros. Trypsin (bovine pancreas, CAS #9002-07-7) was obtained from Alfa Aesar.

Fragaceatoxin C (FraC) monomer expression and purification. pT7-SC1 vector containing His₆-tagged (SEQ ID NO: 101) FraC plasmids were electrochemically inserted into E. coli BL21 (DE3) cells and grown overnight at 37° C. on LB agar plates supplemented with 100 mg/l ampicillin and 1% glucose. Colonies were used to inoculate 200 ml 2×YT medium supplemented with 100 mg/l ampicillin and grown at 37° C. until the optical density at 600 nm (OD₆₀₀) reached 0.6, after which expression was induced using 0.5 mM isopropyl β-D-1-thiogalactopyranoside (IPTG), allowing continued growth overnight at 21° C. Cell pellets were collected by centrifugation (6,000 g, 20 min, 4° C.) and stored at −80° C. for at least one hour. The pellets were resuspended in 10 ml lysis buffer per 50 ml culture, with a lysis buffer consisting of 150 mM NaCl, 15 mM Tris base solution at pH 7.5 supplemented with 1 mM MgCl₂, 2 M Urea, 20 mM imidazole, 0.2 mg/ml lysozyme and 0.2 units/ml DNase. The solution was mixed for 1 hour at room temperature (21° C.) using a rotating mixer at 15 RPM. The cells were fully disrupted by sonification, applying 30 sweeps (duty cycle 30%, output control 3) three times using a Branson Sonifier 450. The lysate was centrifuged at 6000 g for 20 minutes at 4° C. The supernatant was incubated for 1 hour, while under constant rotation (15 RPM), with 100 μL resuspended Ni-NTA resin (resuspended in 150 mM NaCl, 15 mM Tris base at pH 7.5 supplemented with 20 mM imidazole). The solution was loaded onto a prewashed Micro Bio-Spin column (Bio-Rad). The Ni-NTA beads were extensively washed with 20 ml WB (150 mM NaCl, 15 mM Tris base at pH 7.5 supplemented with 20 mM imidazole). The column was inserted into a microtube and spin-dried using a centrifuge (13,300 g, 1 min) in order to remove residual wash buffer. 150 μl of 150 mM NaCl, 15 mM Tris base solution at pH 7.5 supplemented with 300 mM imidazole (EB) was added and left to incubate for 5 minutes before elution. This step was repeated four times to retrieve four fractions containing FraC monomers. The presence and purity of FraC monomers was estimated using SDS-PAGE. Pure fractions were pooled and stored at 4° C. The concentration of FraC monomers was estimated using a Nano Drop 2000 UV-Vis Spectrophotometer (Thermo Scientific) using the elution buffer as blank.

Sphingomyelin-DPhPC liposomes preparation. 25 mg sphingomyelin (Brain, Porcine) was mixed with 25 mg 1,2-diphytanoyl-sn-glycero-3-phosphocholine (DPhPC) and dissolved in 4 ml pentane containing 0.5% v/v ethanol. The lipid mixture was evaporated while turning inside a round bottom flask by application of a hot air stream to create a thin lipid film over the surface of the flask. The film was reconstituted into 10 ml of Sdex buffer (150 mM NaCl, 15 mM tris, pH 7.5) using a sonication bath. The liposome solution (5 mg/ml) was frozen and stored at −20° C.

Fragaceatoxin C oligomerisation. Liposomes were thawed and added to FraC monomers in a lipid to protein mass ratio of 10:1. The mixture was incubated for 30 minutes at 37° C., after which N,N-Dimethyldodecylamine N-oxide (LDAO) was added to a final concentration of 0.6 v/v % to dissolve the liposomes. The solution was diluted 10-fold in 150 mM NaCl supplemented with 15 mM Tris (pH 7.5) and 0.02 v/v % n-Dodecyl 3-D-maltoside (DDM). The diluted solution was combined with 100 μl of Ni-NTA, prewashed using WB2 (150 mM NaCl, 15 mM Tris base, pH 7.5 supplemented with 20 mM imidazole and 0.02 v/v % DDM). The mixture was left to incubate for 30 minutes while mixing under constant rotation (15 RPM). The solution was loaded onto a Micro Bio-Spin column (Bio-Rad), prewashed with 500 μl WB2. The Ni-NTA beads were washed extensively using 10 ml WB2. The column was spin-dried in a microtube using a centrifuge (13,300 g, 1 min) to remove residual wash buffer. 150 μl elution buffer was added onto the column (150 mM NaCl, 15 mM Tris base supplemented with 1M imidazole and 0.02 v/v % DDM) and left to stand for 10 minutes before elution into a clean microtube by centrifugation (13,300 g, 2 min). The oligomers are stable for several months at 4° C. and can be frozen at −80° C. for long-term storage.

Construction of Fragaceatoxin C mutants. Fragaceatoxin C mutant DNA was prepared using the MEGAWHOP method⁶. The megaprimer was constructed using a forward primer synthesized by Integrated DNA Technologies and a T7 reverse primer (5′-GCTAGTTATTGCTCAGCGG-3′ (SEQ ID NO: 46)). Six reactions were performed per mutation in order to receive enough DNA for the second PCR-using 25 μl REDTag® ReadyMix™ PCR Reaction Mix (Sigma-Aldrich) combined with 22 μl PCR grade water (Sigma-Aldrich), 1 μl of each forward and reverse primer and 1 μl His₆-tagged (SEQ ID NO: 101) Fragaceatoxin C template DNA. The PCR protocol consisted of a 90 second denaturation step at 95° C. followed by 30 cycles of denaturation at 95° C. (15 seconds), annealing at 55° C. (15 seconds) and extension at 72° C. (120 seconds). The six PCR reactions were combined and purified using a GeneJET PCR Purification Kit (Thermo Scientific). For the second PCR, 10 μl 5× Phire Buffer (Thermo Scientific) was combined with 1 μl template DNA, 1 μl dNTPs (10 mM), 2 μl megaprimer (first PCR), 35 μl PCR grade water (Sigma-Aldrich) and 1 μl Phire II Hot Start DNA Polymerase (Thermo Scientific). The PCR protocol consisted of an initial pre-denaturing step of 98° C. (30 seconds) followed by 25 cycles of denaturation at 98° C. (5 seconds) and extension at 72° C. (90 seconds). 5.7 μl 5×FD green buffer (Thermo Scientific) and 1 μl Dpn1 enzyme (Thermo Scientific) was added to the PCR mix and let to digest at 37° C. for 1-3 hours. 0.5 μl of the digested product was electrochemically transformed into 50 μl E. cloni 10G® (Lucigen) competent cells and grown on LB agar plates containing 100 mg/l ampicillin and 1% glucose. Single colonies were enriched using a GeneJET Plasmid Miniprep Kit (Thermo Scientific) and the sequence was confirmed using the sequencing service of Macrogen Europe.

### Amino Acid Sequence of His₆-Tagged (SEQ ID NO: 101) Wild Type Fragaceatoxin C.

(SEQ ID NO: 47)
MASADVAGAVIDGAGLGFDVLKTVLEALGNVKRKIAVGIDNESGKT
WTAMNTYFRSGTSDIVLPHKVAHGKALLYNGQKNRGPVATGVVGVI
AYSMSDGNTLAVLFSVPYDYNWYSNWWNVRVYKGQKRADQRMYEEL
YYHRSPFRGDNGWHSRGLGYGLKSRGFMNSSGHAILEIHVTKAGSA
HHHHHH

Unspecific lysozyme digestion. Lysozyme (Carl Roth, From chicken egg white, free from albumin) was dissolved in 8 M urea supplemented with 15 mM Tris (pH 9.5) to a final concentration of 20 mg/ml and left to denature at 95° C. for 5 minutes. 200 μl denatured lysozyme solution was incubated for 30 minutes at 37° C. with 20 mM dithiothreitol (DTT), to reduce the cysteine residues. Iodoacetamide (IAA) was added to the mixture, to react with reduced cysteines, with a final concentration of 45 mM and incubated in the dark for 30 minutes at room temperature. The mixture was diluted 5× with 100 mM Tris (pH 8.5) and trypsin (Alfa Aesar™ Trypsin, bovine pancreas) was added in a ratio of 1:50 (trypsin:protein). The mixture was left to digest overnight (˜18 hours) at 37° C. In order to denature and deactivate any remaining trypsin, the next day, the final mix was denatured at 95° C. for 10 minutes and HCl was added to lower the pH (approximately pH 4). The mixture was then frozen at −20° C. until use.

Planar lipid bilayer electrophysiological recordings. The electrophysiology chamber consisted of two compartments separated by a 25 μm thick Teflon (Goodfellow Cambridge Ltd) membrane. The Teflon membrane contained an aperture with a diameter of approximately 100-200 μm. Lipid membranes were formed by first applying 5 μl of 5% hexadecane (Sigma Aldrich) in pentane (Sigma Aldrich) to the Teflon membrane, near the aperture. The pentane was left to dry and 400 μl of buffer (1 M KCl, 50 mM citric acid, titrated with bis-tris propane to pH 3.8) was added to both sides. 20 μl of a 6.25 mg/ml solution of DPhPC dissolved in pentane was added on top of the buffer on each side of the chamber. The chamber was left to dry for −2 minutes to allow evaporation of pentane. Silver/silver chloride electrodes were attached to each compartment. The cis compartment was connected to the ground electrode and the trans was connected to the working electrode. Planar lipid bilayers were created using the Langmuir-Blodgett method described by Maglia et al.⁷. The orientation of FraC nanopores was determined by the asymmetry of the current-voltage relationship. A baseline of 2 minutes was recorded for each of the pores recorded. Analytes were added to the cis compartment of the chamber.

Data recording. Recordings of ionic currents were obtained using an Axopatch 200B (Axon Instruments) combined with a Digidata 1550B A/D converter (Axon instruments), similar to preceding work^{1,2}. The sampling frequency was set at 50 kHz for analyte recordings, the analogue Bessel filter was set at 10 kHz. Data was recorded using Clampex 10 (Molecular Devices).

### Standard Data Analysis and Event Detection.

A number of well-known means of analysing the stepwise current blockades measured from nanopore electrophysiology are known in the art, and various methods can be employed on the events types we observe to extract useful data, which include but are not limited to blockade magnitude, blockade duration, blockade shape, blockade noise, other sub-features of the blockades (such as ministeps, etc).

For basic data analysis a custom Python script was employed to analyse the raw electrical data. The open pore current (Iₒ) and standard deviation of all traces was determined by calculating the mean current of 3 independent measurements, bootstrapped for 100 iterations of 10 second snippets for each measurement. For event detection, the baseline current and standard error of the recorded traces were determined from a full current histogram of the blank nanopore measurement containing no analytes. The value for the baseline was then used to determine the events when analyte was added. All data points above the baseline current and standard error that are separated by at least two times the sampling periods are detected as events. The excluded current (Iₑₓ%) of each event was calculated as the difference between the open-pore current Jo and the blockade current Ib, over the open-pore current Io (Iex %=[Io−Ib]/Io).

### Impartial Event Detection.

An impartial event detector method was employed to improve analyses. We found that short lived events—with a dwell time near the sampling frequency-tend to form a spike or Gaussian profile due to under sampling and filtering effects, while longer events follow a flat-top shape. Therefore, we introduced a parameter describing the shape of current blockades in order to impartially compare the performance of mutant pores. We assume that the profile of ionic current blockades can be described by a generalized flat-top normal distribution function (gNDF, Equation 3). Each observed block was fit to equation 1 using least-squares fitting, due to the non-polynomial nature of the function.

$$f ( x ) = Δ {I}_{B} * exp ( - {( \frac{{( x - μ )}^{2}}{2 {σ}^{2}} )}^{β} ) + {I}_{O} for β > 0 \qquad ( 3 )$$

Where μ is the events centre in the time domain with variance σ² and ΔI_{B} is the current difference (pA) between the baseline (I_{O}) and the event maximum. The variable β describes the shape of the function and can take any real number larger than zero. If β is less than one but larger than zero, the shape of the function is a spike. If β is equal to one, the function is equal to the normal distribution function. When f is larger than one, the function starts to follow a rectangular-flat-top-profile. Advantageously, the variable β can also be used to assess the quality of individual events in the following way. Events with a β<1 are mostly events that are too short-lived to accurately measure the ionic current blockade. Therefore, only those events with a β≥1 should be regarded as more accurate measurements of peptides. Similarly, we distinguish events with β≥10, since these events-having a flat-top shape-permit an more accurate estimation of the blocked current. The gNDF fit also permits an estimation of the dwell time of an event by taking the full width at half maximum (FWHM) of the gNDF (Equation 4). Estimation of the dwell time using this equation is advantageous, because it allows the treatment of this parameters as continuous rather than discrete, which is the case if the number of data points are counted within the event.

$$F W H M = 2 σ \sqrt{{2}^{β} \sqrt{ln 2}} \qquad ( 4 )$$

Where σ equals the square root of the variance (σ², Equation 3) and β describes the shape parameter.

Spectral matching. Several of the residual current spectra we obtain are expected to contain random events induced by factors other than the analyte (gating), so in order to reduce baseline sloping and to maintain high sensitivity, we utilize the squared first derivative Euclidean cosine correlation (Equation 5)⁹. This comparison is sensitive to the position of the peaks observed in the spectra, but not as sensitive to a shifting baseline.

$$Correlation = \frac{{( {\sum }_{i} Δ {A}_{1 , i} Δ {A}_{2 , i} )}^{2}}{{\sum }_{i} Δ {A}_{1 , i}^{2} {\sum }_{i} Δ {A}_{2 , i}^{2}} \qquad ( 5 )$$

Where A₁ and A₂ equal the vectors of excluded current counts and A_{1,i} and A_{2,i} represent the individual bins of the excluded current spectrum⁹. In a more detailed description, we set A₁ and A₂ as the vector of counts we observe for each residual current bin (e.g. Aₙ=counts (40-41%), counts (41-42%), . . . , counts (94-95%)). ΔAₙ is the derivative of Aₙ (difference between bins). In the numerator, we multiply each element ΔAₙ with the corresponding ΔAₙ of the comparing spectrum and take the squared sum of all items. In the denominator, we take the squared sum of each element in ΔAₙ and multiply that with the squared sum of each element in the spectrum we want to compare. So, if the two vectors A₁ and A₂ are equal, the correlation is 1, else it is less than 1, and because the derivative of A₁ and A₂ is taken, linear baseline sloping is less impactful.

We performed hierarchal clustering using the Ward distance as implemented in SciPy version 1.4.1. on the resulting correlation coefficients to determine which spectra are most similar¹⁰. In essence, this metric orders the data in such a way that the variance between neighbours is minimal, therefore building a map of similar spectra.

### Example 1: Fragaceatoxin C Mutant Screening

Mutations of FraC:

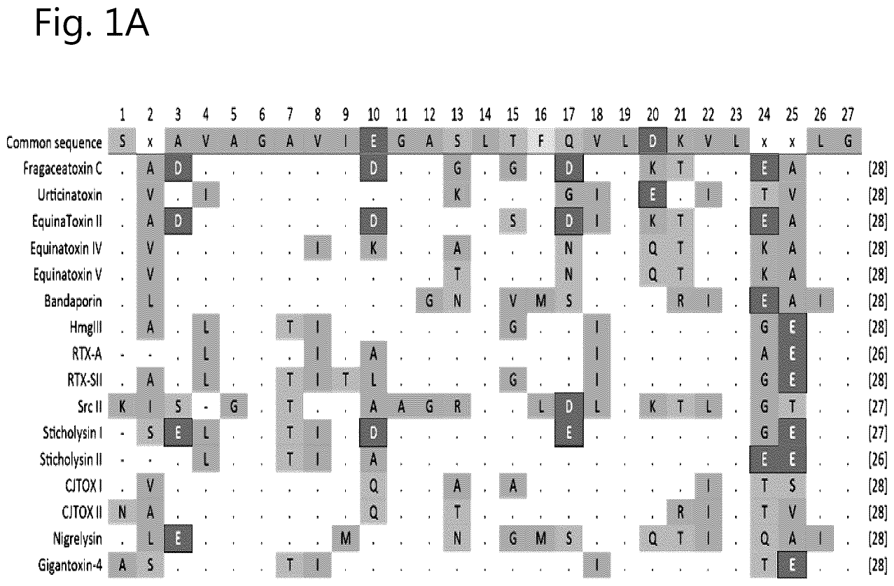

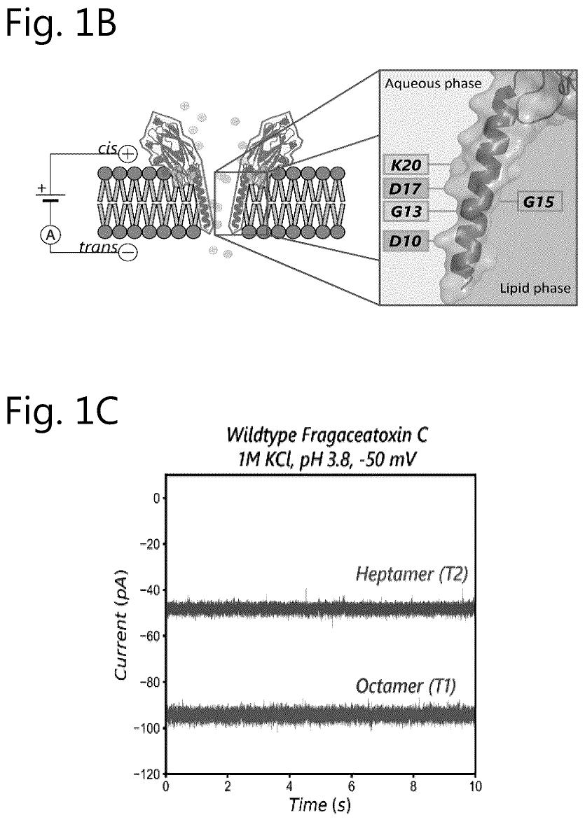

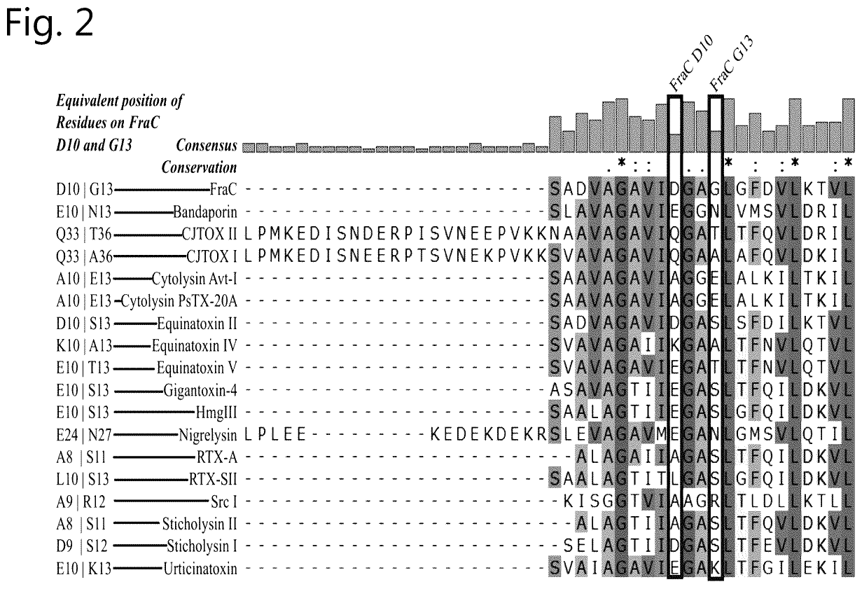

The sequence of WtFraC from the sea anemone Actinia fragacea was aligned with other actinoporins (FIG. 1A) to identify sequence homology. A number of generally non-conserved positions were identified that would be more amenable to mutation, including D10, G13, G15, D17 and K20 (FIG. 1i). These positions were engineered into different mutations to improve the ability of the pores to detect and discriminate different peptides.

At position D10, mutations to arginine (R) and Glycine (G) were introduced to test changes in electro-osmotic capture of analytes. Each of the positions near the recognition site (G13), was modified to a basic residue (K, R or H) or acidic residue (D or E) as well as amino acids with neutral (G or Q) or aromatic (W,Y and F) groups. In FraC a glycine residue is positioned at residue 15—while the most common amino acid in other actinoporins is a threonine. Mutation G15T was introduced to test whether increased hydrophobic mutations facing outwards into the membrane would stabilize and improve the behavior of FraC pores.

Sequence alignment (FIG. 1A) shows a pair of opposite charges commonly at positions 20/21, therefore, two mutants that have the same characteristics, T21D and the double-mutant K20D/T21K, were constructed. A change of charge on position 20 by introducing a glutamic acid (K20D) was also tested.

### Oligomeric Forms:

WtFraC can exist in three oligomeric forms, that are predicted to correspond to octamers, heptamers and hexamers. We tested octameric pores (or type I pores, T1) and heptameric pores (or type II pores, T2), and hexameric pores (or type III pores, T3). Octameric oligomers were identified as the nanopores with the highest conductance. Several mutations significantly reduced the open pore current (I₀) relative to WtFraC-T1 (95±1 pA), some to an extent that the I₀ resembled WtFraC-T2 (47±3 pA). Notably, decreased I₀ were observed when residues with a larger volume were introduced, particularly for the aromatic residues (W/F/Y) introduced on position 13 (I₀=64±8 pA, 77±4 pA and 82±3 pA, respectively), suggesting that smaller recognition region can be achieved, which can be advantageous for detecting smaller analytes such as small peptides. The introduction of a threonine residue on position 15 increased the open-pore current I₀ flowing through the pore (100±3 pA), which is a useful property in nanopore analysis as the increased current is generally more sensitive to changes due to analyte binding.

### Peptide Mixtures:

In order to ensure a fair comparison between pores, a mixture of peptides was generated from the non-specific tryptic digestion of lysozyme (Gallus-Gallus). We used trypsin or other proteases such as chymotrypsin or Lys-C protease. The use of trypsin might be advantageous because it cleaves preferentially after a K/R amino acid and as most peptides will have a positive charge next to the zwitterionic charges on the peptide, yielding a net charge of +1 under the low pH conditions employed. All pores were tested with the same proteolytic mixture.

### Blockade Event Analysis:

Events arising from nanopore current blockades were analysed with a flat-top shape fitted using a least-squared Levenberg-Marquardt method and a generalized flat-top normal distribution function. The fit results in a p value that can classify the events as either a spike with β<1, a normal distribution β=1 or flat-top distribution β>1. All events with β>1 were used in subsequent analyses. For each blockade a number of characteristic metrics are extracted. These include the excluded current (Iₑₓ%), which is the percentage of the current that is blocked during a translocation event relative to the open pore current (Iₑₓ%=[Io−Ib]/Io)), the duration (termed dwell time) of the blockade, the shape of the blockade, the noise in the blockade current etc.

### Experimental Conditions

Peptide capture and discrimination in FraC nanopores was studied under a wide range of conditions. Peptide capture was observed over a wide range of voltages, for example from lower voltages of +−10 mV through to +−200 mV. The majority of sensing was carried out at +−50 mV to +−100 mV as this was generally found to be optimal for peptide capture and characterization. Peptide detection can be observed over a wide range of salt types, concentrations and asymmetries across the membrane, all of which in combination with the pore type can alter the capture and detection properties of the system. Preferred salt conditions are about 1 M KCl (or NaCl or LiCl) at pH≤4.5 (eg. pH 3.8). Results:

Wild Type FraC-T1 and wild type FraC-T2 captured peptides at a frequency of about 10-13 events·s⁻¹ under pH 3.8 conditions. When the charge at position 10 or 17 was removed (D10G-FraC-T1 or D17Q-FraC-T1 mutation), the capture frequency was reduced by about 3.4 times and about 7.2 times relative to WtFraC-T. It has been shown that the electro osmotic flow (EOF) is a critical component for efficient capturing of peptides in the nanopore, and can act with or against to electrophoretic forces acting on analytes. It has also been shown that the strength and direction of the EOF is dependent on charges in the constriction site (Table 4). Under low pH, which partially protonates water facing residues and generally increasing the net positive charge inside the pore (increasing anion selectivity), we found that the native negative residues in wild type FraC result in almost zero net ion selectivity (Table 4) and thus almost zero net electro-osmotic flux across the nanopore (versus very high cation selectivity at pH 7.5). Removing the negative charge at position 10 further increases the anion selectivity at low pH, creating a stronger EOF component acting against the capture of mostly positively charged peptides, hence resulting in lower capture efficiency. Furthermore, pores with a positively charge constriction, such as D10R-FraC-T1, showed a destabilized baseline current under an applied bias of −50 mV, but stable under +50 mV, thereby behaving opposite to WtFraC. However, D10R mutations exhibited good capture of peptide analytes in the cis chamber at positive applied voltage (exhibiting similar capture to that of native D10 in WT under negative voltage). The increased capture under this polarity is the result of a strong net anion-selective electro-osmotic bias (flowing from cis to trans) that is created by the positive mutation, which is dominant versus the weaker electrophoretic force acting against peptide capture at this polarity.

Removing the charge on residue K20 by substitution to glutamine increases the capture frequency by 1.4 times relative to wild type Frac. Replacing the charge of K20 by introducing an aspartic acid reduced the capture frequency by 1.5 times relative to wild type FraC. These relatively small changes illustrate how the EOF can be fine-tuned to control the capture frequency and/or the event residence (dwell) time.

Interestingly, we find that the introduction an aromatic residue (Y, F or W) increases the capture frequency by about 4 times relative to the wild-type FraC-T1 and FraC-T2 pores for all three mutations.

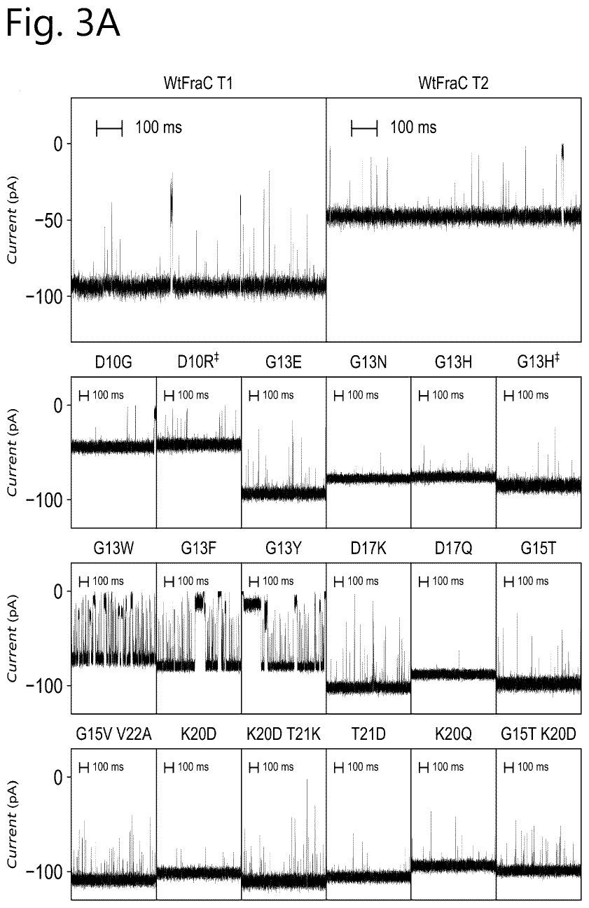

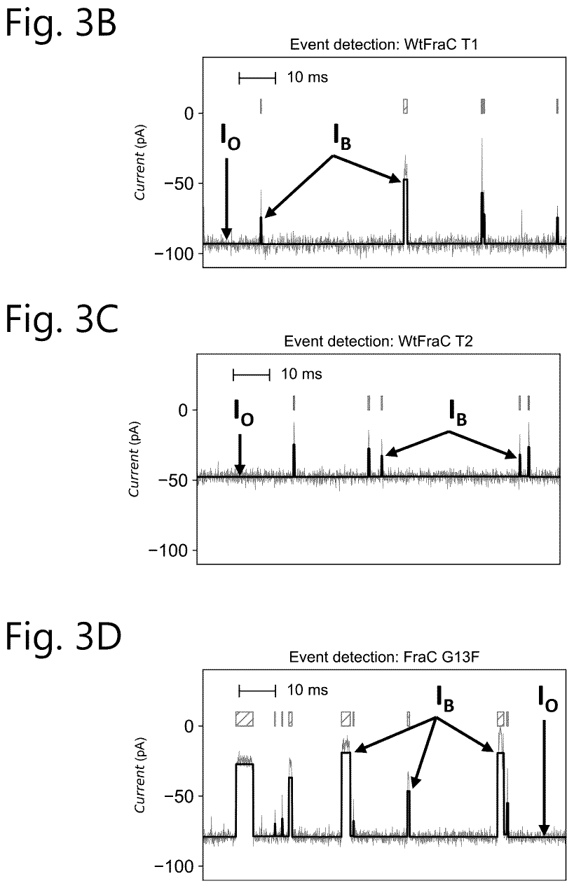

Furthermore, we find that the aromatic mutations also increase the duration of the peptide event blockades in the nanopores. Most of the blockades in pores with an aromatic residue on G13, were flat-top shaped with relatively long dwell times (e.g. FIG. 3D). In fact, the median dwell time of events in these aromatic pores is increased to 0.32±0.06 ms, 0.18±0.03 ms and 0.22±0.06 ms for G13Y-FraC-T1, G13F-FraC-T1 and G13W-FraC-T1 respectively compared to 0.09±0.06 ms for WtFraC-T1 and 0.10±0.01 ms for WtFraC-T2.

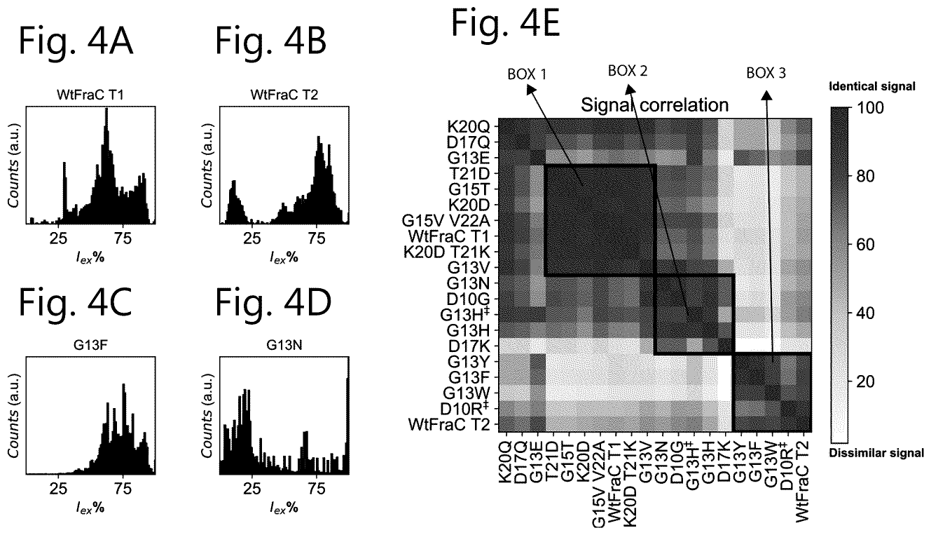

In order to compare the different mutants, we constructed the excluded current spectrum (shown for 4 pores in FIG. 4A-D) by creating a histogram of the excluded currents (Iₑₓ%) using all events with β>1 (5 kHz Gaussian filter, see methods). We normalized the spectra and observe distinct patterns for WtFraC-T1 and T2 (FIG. 4A/B) with sharp gaussian shaped peaks for G13F-FraC-T1 (FIG. 4C). The majority of peaks of G13N-FraC-T1 were at low Iex % (FIG. 4D), reflecting the faster translocation of peptides across the nanopore. We compared the excluded current spectra using a point-to-point spectral matching algorithm, using the excluded current spectrum where 40%<Iₑₓ%<95%.

### Example 2: Fragaceatoxin C Mutant Characterization

We selected five mutants for further characterization, namely: G15T-FraC-T1, as it is comparable to WtFraC-T1 with a slightly increased I₀, K20D-FraC-T1 as it had one of the higher SNRs and good capture frequency and the aromatic mutations of at G13 (G13Y/F/W-FraC-T1) for their increased dwell times compared to WtFraC-T2 and capture frequency. For the characterization of these pores we used a mixture of well-defined peptides (i.e. the mixture was made by adding the individual peptides at equimolar concentrations). The mixture consisted of four peptides: Angiotensinogen (DRVYIIIPFHLVIHN (SEQ ID NO: 48), 1758.9 Da, charge=+3.96), Angiotensin 1 (DRVYIHIPFHL (SEQ ID NO: 49), 1296.5 Da, charge=+2.96), Angiotensin 3 (RVYIHPF (SEQ ID NO: 50), 931.1 Da, charge=+2.16) and Angiotensin 4 (VYIIIPF (SEQ ID NO: 51), 774.9 Da, charge=+1.16) abbreviated as Angiotensinogen, Ang-I, Ang-III and Ang-IV respectively. The resolution of the nanopores was quantified by measuring the separation between peptides using the difference between the peak centers and their mean standard deviation as shown in Equation 1 and 2.

$$\overline{σ} = \frac{( {σ}_{1} + {σ}_{2} )}{2} \qquad ( 1 )$$

$${R}_{s} = \frac{{μ}_{1} - {μ}_{2}}{\overline{σ}} = \frac{2 \cdot ( {μ}_{1} - {μ}_{2} )}{{σ}_{1} + {σ}_{2}} \qquad ( 2 )$$

Where Rₛ is resolution, μ₁ and μ₂ are the peak centers with standard deviation σ₁ and σ₂ respectively. If Rₛ<2, the difference between the peak centers is less than twice the average standard deviation. Therefore, no baseline separation is achieved. To achieve an overlap of less than 5%, a Rs≥4 is required, that is, the difference between the peak centers is equal or bigger than twice the average standard deviation of the peaks, thus we can consider them separated. Larger values of R indicate a better separation (Table 2).

TABLE 2
The differences between peptide peak centers (ΔIex %) and the observed baseline separation (Rs).

| | | | K20D- | G15T- | G13F- | G13Y- | G13W- |
|---|---|---|---|---|---|---|---|
| MW: 1759-931 | FraC-T1 | FraC-T2 | FraC-T1 | WtFraC-T1 | FraC-T1 | FraC-T1 | FraC-T1 |
| ΔIₑₓ % | 8.8 ± 0.7% | 18 ± 3% | 14 ± 6% | 12 ± 5% | 9.2 ± 0.3% | 9.1 ± 0.7% | 5.0 ± 0.3% |
| (Ang-IV − | | | | | | | |
| Ang-III) | | | | | | | |
| ΔIₑₓ % | 17 ± 2% | 12.3 ± 0.5% | 15 ± 1% | 17 ± 2% | 24 ± 1% | 22 ± 1% | 19.9 ± 0.2% |
| (Ang-III − | | | | | | | |
| Ang-I) | | | | | | | |
| ΔIₑₓ % | 19.0 ± 0.2% | 9.3 ± 0.3% | 16.2 ± 0.4% | 19.0 ± 0.3% | 10 ± 1% | 6.1 ± 0.8% | 6.4 ± 0.2% |
| (Ang-I − | | | | | | | |
| Pre-Ang) | | | | | | | |
| Rₛ | 2.1 ± 0.7 | 4.1 ± 1.2 | 2.6 ± 1.4 | 2.0 ± 0.5 | 4.6 ± 0.5 | 4.4 ± 1.1 | 3.6 ± 0.4 |
| (Ang-IV − | | | | | | | |
| Ang-III) | | | | | | | |
| Rₛ | 3.5 ± 0.5 | 4.2 ± 0.5 | 2.3 ± 0.2 | 3.3 ± 0.4 | 12.1 ± 4.3 | 11.8 ± 2.9 | 19.1 ± 1.7 |
| (Ang-III − | | | | | | | |
| Ang-I) | | | | | | | |
| Rₛ | 4.1 ± 0.3 | 4.0 ± 0.3 | 3.2 ± 0.5 | 4.6 ± 0.2 | 6.1 ± 2.3 | 4.0 ± 0.7 | 7.2 ± 1.2 |
| (Ang-I − | | | | | | | |
| Pre-Ang) | | | | | | | |

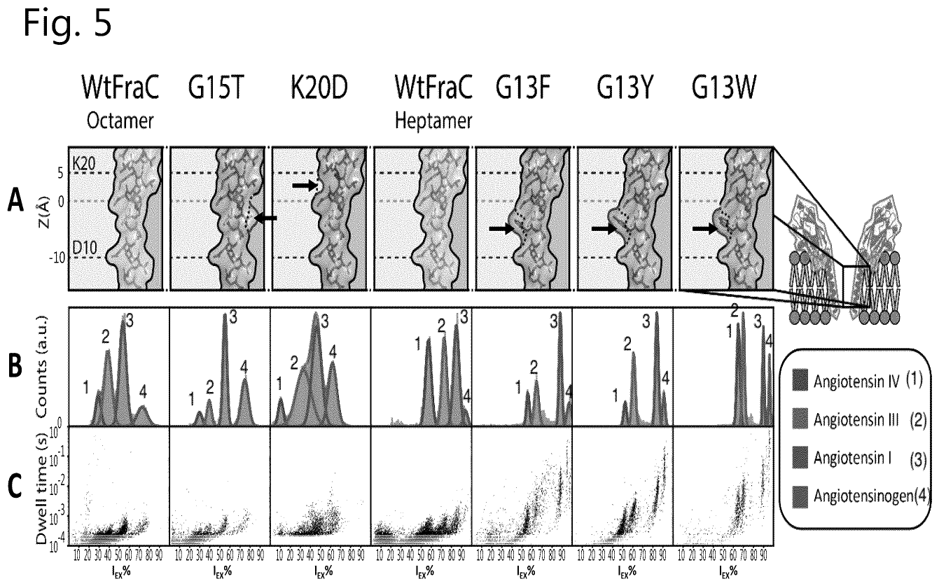

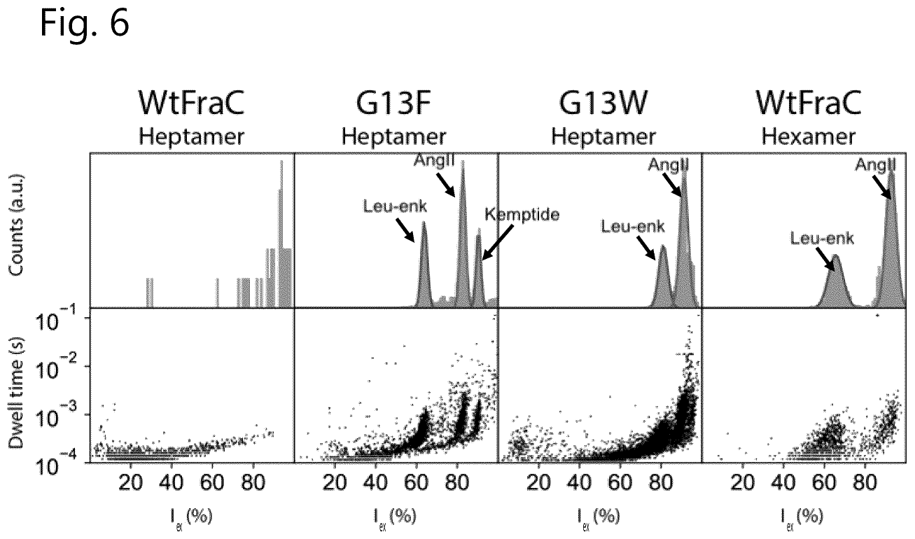

FIG. 5 shows the comparison between WtFraC-T2 and the selected engineered FraC pores. The aromatic pores G13F/Y/W showed marked improvement in the ability to discriminate between the peptides. The aromatic pores exhibit significantly longer blockade event durations versus WtFraC-T2. Longer duration events (with more raw data points at a given acquisition frequency) enable the amplitude of the excluded current for the individual event blockades to be determined to a higher accuracy. This can at least in part account for the reduced spread in the excluded current observed for each peptide cluster for the aromatic pores.

### Example 3: Peptide Analysis with T2 Nanopores

We tested the resolution of aromatic heptameric (T2) nanopores, and compared to hexameric (T3) WtFraC-T3 and WtFraC-T2 nanopores using Leucine-enkephalin (Leu-enk, YGGFL (SEQ ID NO: 36), 555.6 Da), Angiotensin II (4-8) {Ang-II(4-8), YIHIPF (SEQ ID NO: 37), 675.8 Da}, and Kemptide (LRRASLG (SEQ ID NO: 38), 771.9 Da). For WtFraC-T3 we use a FraC version with two altered membrane-interfacing modifications, W112S-W116S, which allowed forming hexameric nanopores. WtFraC-T2 showed no blockades (FIG. 5), suggesting that the majority of peptides translocated through the pore undetected. FraC-T3 and G13W-FraC-T2 showed leucine-enkephalin and angiotensin II (4-8) blockades, while kemptide blockades were not observed. This is surprising, considering kemptide has higher molecular weight than leucine-enkephalin and angiotensin II (4-8). Possibly, the two arginine residues in the kemptide induce a fast electrophoretic translocation across these nanopores. Interestingly, we found that kemptide induced blockades to G13F-FraC-T2, indicating that this aromatic modification is of paramount importance to detect this class of peptides. A likely explanation is that cation-7c interactions between the ring of phenyl alanine residues and the two arginine residues are crucial to reduce the residence time of the peptide inside the nanopore.

TABLE 3
The differences between peptide peak centers (ΔIₑₓ %)
and the observed baseline separation (Rₛ).

| | FraC- | | G13F-FraC- | G13W-FraC- |
|---|---|---|---|---|
| MW: 772-556 | T2 | FraC-T3 | T2 | T2 |
| ΔIₑₓ % | N.O. | 27.6 ± 0.8% | 19.1 ± 0.1% | 10.6 ± 0.8% |
| (Leu-enk - Ang-II (4-8)) | | | | |
| ΔIₑₓ % | N.O. | N.O. | 6 ± 2% | N.O. |
| (Ang-II (4-8) - Kemptide) | | | | |
| ΔIₑₓ % | N.O. | 5 ± 1 | 11 ± 2 | 3 ± 2 |
| (Leu-enk - Ang-II (4-8)) | | | | |
| Rₛ | N.O. | N.O. | 3 ± 2 | N.O. |
| (Ang-II (4-8) - Kemptide) | | | | |

### Example 4: Analytical System Comprising Nanopores

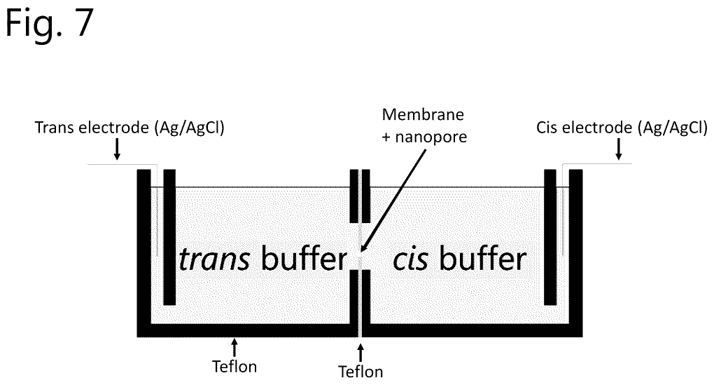

Nanopores are nanometer sized apertures in thin membranes that detect analytes moving through the aperture. Aₙ exemplary analytical system of the invention is schematically depicted in FIG. 7. It consists of two chambers filled with an electrolyte solution, separated by a membrane. The chambers are connected via a nanopore that is formed in the membrane. When a potential is applied across the membrane via the electrodes in either chamber, ions will move through the pore generating a small ionic current that is amplified and measured. When an analyte enters the nanopore, the ionic current flowing through the open-pore is altered due to the displacement of ions by the analyte, typically resulting in a reduction in ionic current (blockade event). The characteristics of the current blockade (eg. the magnitude, duration, shape, noise, etc) are dependent on the nature of the analyte captured and the conditions (eg. applied potential, buffer conditions, temperature, etc), and can be used to inform on the properties of the captured analyte.

### Example 5: FraC Nanopore as a Next Generation Single-Molecule Protein Analyser

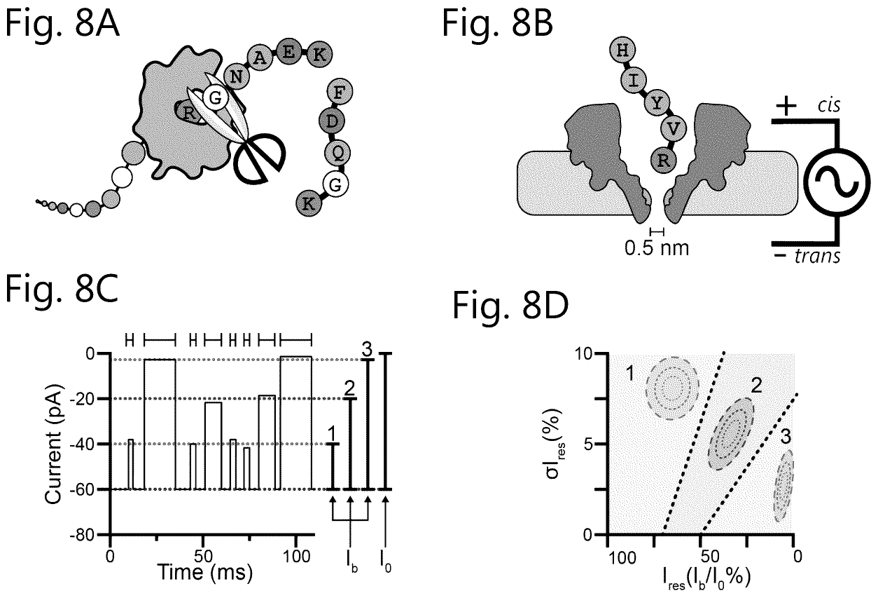

This example demonstrates that an engineered sub-nanometer biological nanopore of a mutant Fragaceatoxin C (FraC) is able to identify multiple trypsin digested proteins. By calibration through several synthetic peptides, a relation between the residual current spectrum and mass-spectrum could be found, thus allowing for protein identification. FIG. 8 illustrates the concept of such “bottom-up” nanopore-based proteomics.

Protein digestion. 100 μg of protein stock was taken and the volume was adjusted to 50 μl using 20 mM Tris buffer (pH 7.5). A final concentration of 20 mM dithiothreitol (DTT) was added to reduce any disulphide bonds. The sample was incubated at 37° C. for 15 minutes followed by a denaturing step at 95° C. for 15 minutes. Afterwards, a 20 mM iodoacetamide (IAA) was added and the sample was left to incubate for 15 minutes at room temperature in the dark in order to alkylate the reduced cysteine residues. Finally, the total volume was adjusted to 100 μl using 100 mM Tris Buffer (pH 8.5).

For the tryptic digestion we used a kit purchased from Sigma-Aldrich, containing proteomics grade trypsin. 50 μl of sample (containing 50 μg of protein) was added to 1 μg of mass-spec grade trypsin (1:50 enzyme:protein ratio) and the sample was subsequently incubated overnight at 37° C. Finally, large (>2000 Da) peptides were removed using centrifugal filters with a molecular weight cut-off of 3000 Da (Amicon). Filtered samples were stored in −20° C. prior to use.

Trypsin is a sequence dependent protease, and cuts mainly at the carboxyl side chain of arginine (R) and lysine (K) residues unless they are followed by proline (P). Trypsination of a given protein therefore results in a peptide mixture containing a specific set of peptide fragments from specific cutting, combined with some level of other peptide fragments resulting from incomplete digestion or off-target cutting.

Expression of proteins for tryptic digestion. Five model proteins: DHFR (dihydrofolate reductase), BSA (Bovine serum albumin, Sigma-Aldrich), PAN (PAN unfoldase), ThpA (Thiamine binding protein) and HMWI_Act (C-terminal fragment of Haemophilus influenzae high-molecular weight adhesin protein, residues 1205-1536) were expressed and/or purified for the purpose of testing the nanopore sensors.

Protein expression of DHFR PAN ThpA/HMWI_Act: All proteins were expressed via similar protocols. Briefly, plasmid containing the gene of interest, was electrochemically transformed into BL21(DE3) competent Escherichia coli cells. The cells were grown overnight at 37° C. on LB agar plates supplemented with 100 mg/L ampicillin and 1% glucose. On the next day, grown LB plates were solubilized into 200 mL 2×YT medium, supplemented with 100 mg/L ampicillin. Cultures were grown under constant shaking at 37° C. until an optical density (OD₆₀₀) of 0.6 was reached. Afterwards, 0.5 mM isopropyl β-D-1-thiogalactopyranoside was added for induction and growth continued overnight at 21° C. Bacterial cells were pelleted using centrifugation and stored for at least one hour at −80° C.

Protein Purification of DHFR/PAN/ThpA/HMWI_Act: Cell pellets were processed by first resuspending in lysis buffer and lysing by sonication (Branson Sonifier 450) in the presence of a protease inhibitor cocktail (Roche). Cell debris was removed by centrifugation and supernatant was processed via Ni-affinity chromatography columns to recover the purified protein fractions. For PAN an additional purification was performed, purifying the protein via anion exchange using HiTrap Q HP anion exchange columns (GE Healthcare Life Sciences). Purity was confirmed by SDS-PAGE and the fractions with highest protein concentration were combined and concentrated using a 10 kDa MWCO spin filter (Amicon). For HMW1Act, fractions containing protein of interest were collected and dialyzed using SnakeSkin dialysis system (MWCO 10 kDa, Thermo Fischer Scientific) against storage buffer (50 mM HEPES, 100 mM NaCl, 10% glycerol, pH 7.5). After dialysis protein was aliquoted and stored at −80° C. until further use.

Protein purification of BSA: BSA was purchased from Acros Organics. The purity of BSA was increased using anion exchange chromatography (Äkta pure) by processing 10 mg BSA (in 1 ml 50 mM Tris, pH 7.5) on a HiTrap Q HP anion exchange column (GE Healthcare Life Sciences). Eluted protein fractions were assessed by SDS-PAGE and the fractions with highest protein concentration were combined and concentrated using a 10 kDa MWCO spin filter (Amicon).

### Results

Detection of a model protein digest. The detection and identification of proteins using, (standard) mass spectrometry based, techniques relies heavily on the fingerprinting of (tryptic) peptides. To mimic a properly digested protein, we employed a model peptide system containing 7 synthetic peptides with a mass between 700 and 1700 Da (Sigma Aldrich and Genscript) that would be predicted to result from complete trypsination of lysozyme, i.e. the protein is cleaved in-silico at all arginine (R) and lysine (K) residues unless they are followed by proline (P).

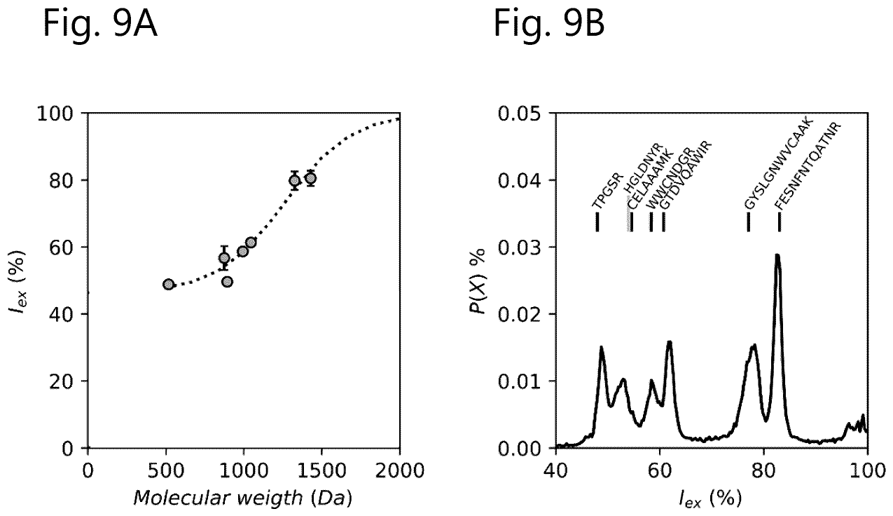

The 7 model peptides were individually added to separate nanopore experiments (G13F-FraC-T1 pores, 1M KCl, pH 3.8, −50 mV), generating a unique cluster of events when plotted by excluded current and dwell time. For each single experiment the average excluded current for the event blockades was calculated by fitting a gaussian to histograms of the clustered events. The average excluded current for each peptide type was calculated by averaging across n>3 experiments performed on each peptide. A strong correlation between the molecular weight of the peptides and their respective average excluded current blockade was observed (FIG. 9A). The data were fitted with a logistic function (Equation 1, FIG. 9A), which enables prediction of peptide mass from excluded current measurements.

$$f ( x ) = a + \frac{1 - a}{1 + exp ( - ( \frac{1}{k} ) * ( x - μ ) )} \qquad ( 1 )$$

Where a is the offset, k represent the width and μ is the inflection point.

FIG. 9B shows a histogram of excluded current blockade events measured from a mixture of all 7 model peptides in the nanopore system (G13F-FraC-T1 pores, 1M KCl, pH 3.8, −50 mV). The peaks are labelled according to the predictions from the logistic function, and match the same excluded current position observed in the individual experiments.

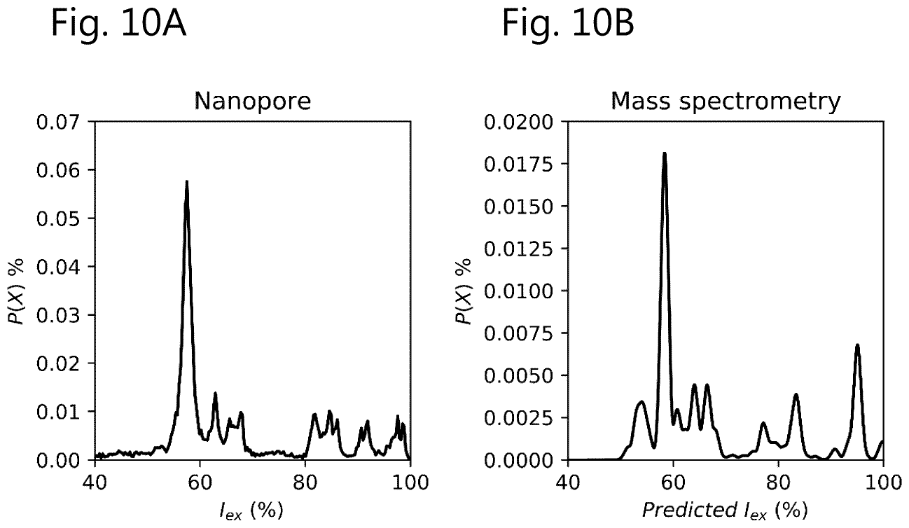

Detection of digested Lysozyme protein and comparison with Mass Spectrometry. Lysozyme protein was digested via trypsination as described above. The resulting peptide fragment mixture was then analyzed both using nanopore sensing (G13F-FraC-T1 pores, 1M KCl, pH 3.8, −50 mV) and with Mass Spectrometry (LC ESI-MS). A histogram of the excluded current blockades measured from the mixture using the nanopores is plotted in FIG. 10A. For the purposes of comparison the mass data obtained from the Mass Spectrometry spectrum was transformed onto an pseudo excluded current axis using the predictions from the logistic fit parameters determined from Equation 1 (FIG. 10B). Notably, although the methods cannot be directly compared due to differences in detection efficiency for example, we observed a significant correlation between the observed electrospray ionization (ESI) mass-spectrum and the nanopore mass spectrum.

### Detection of Trypsin Digested Proteins

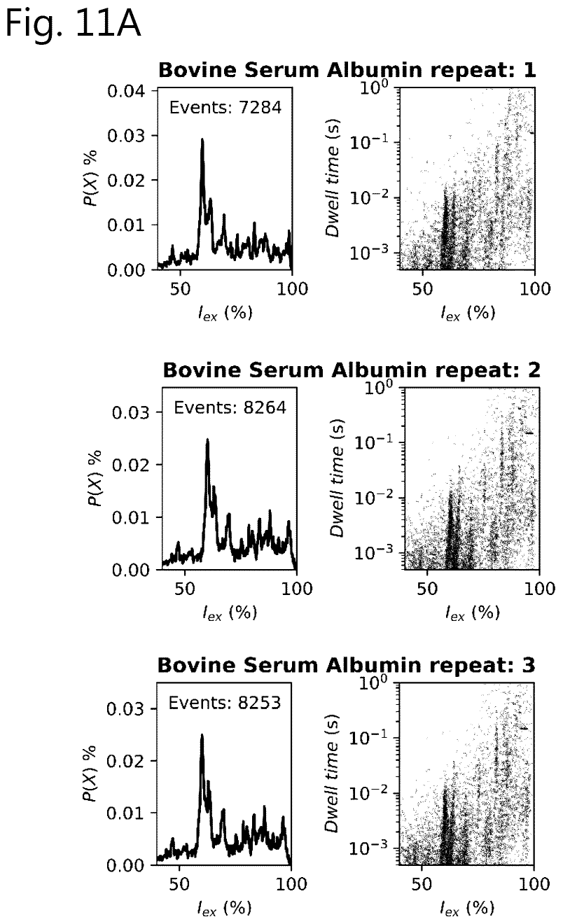

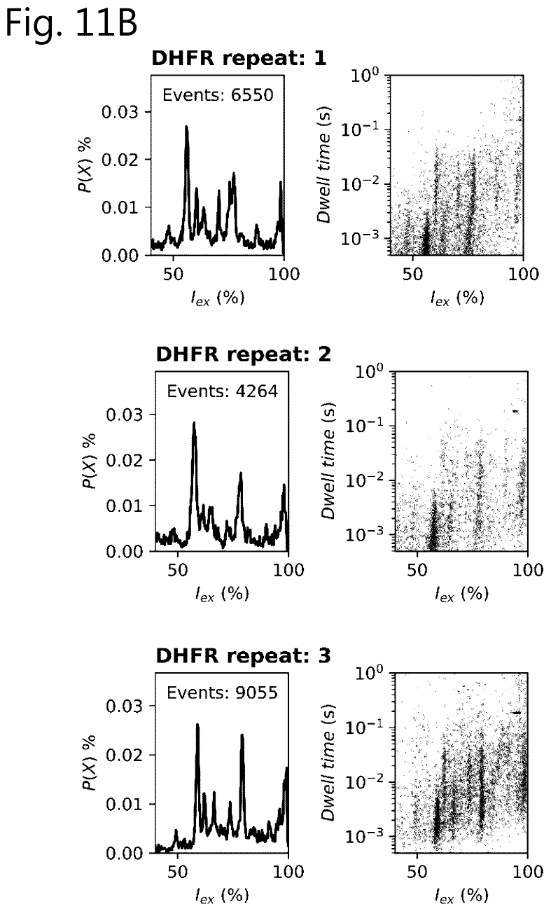

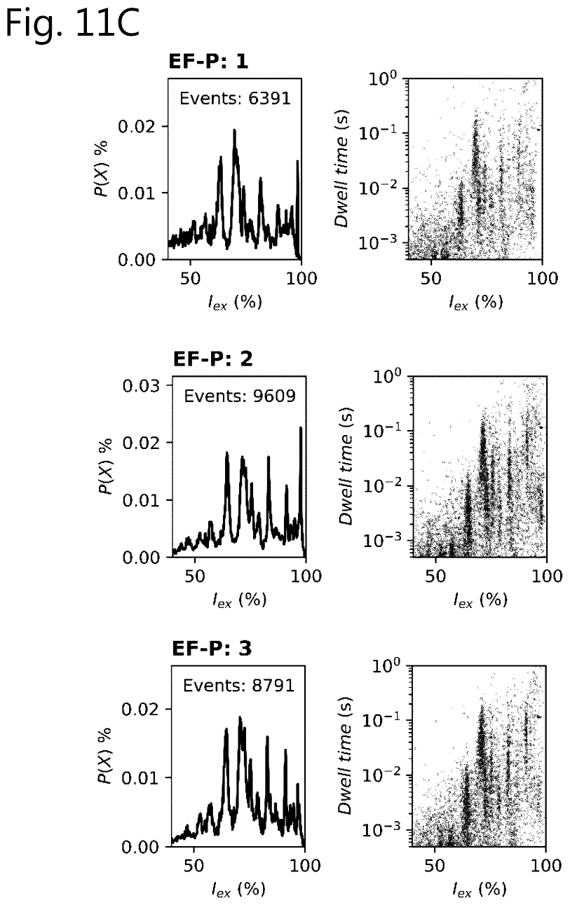

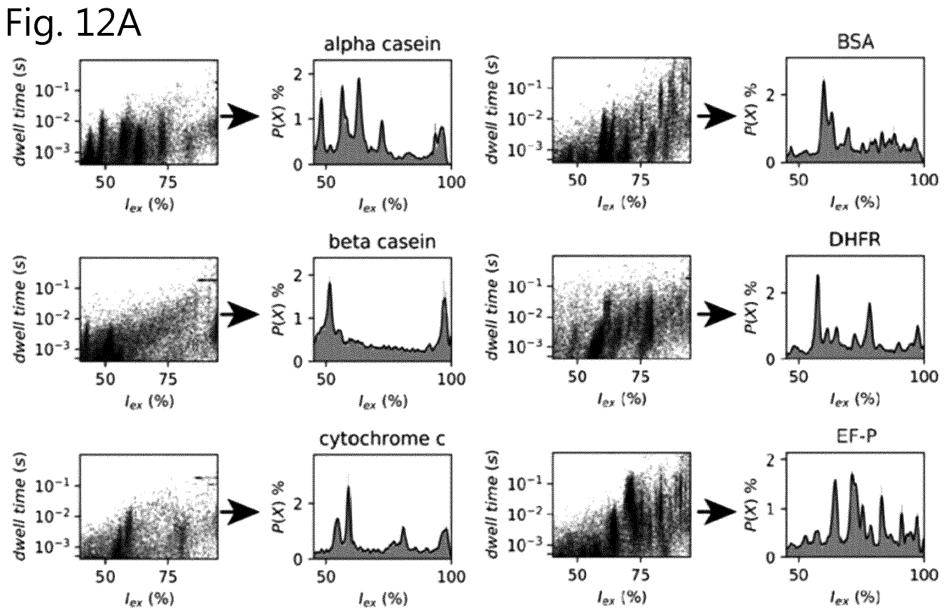

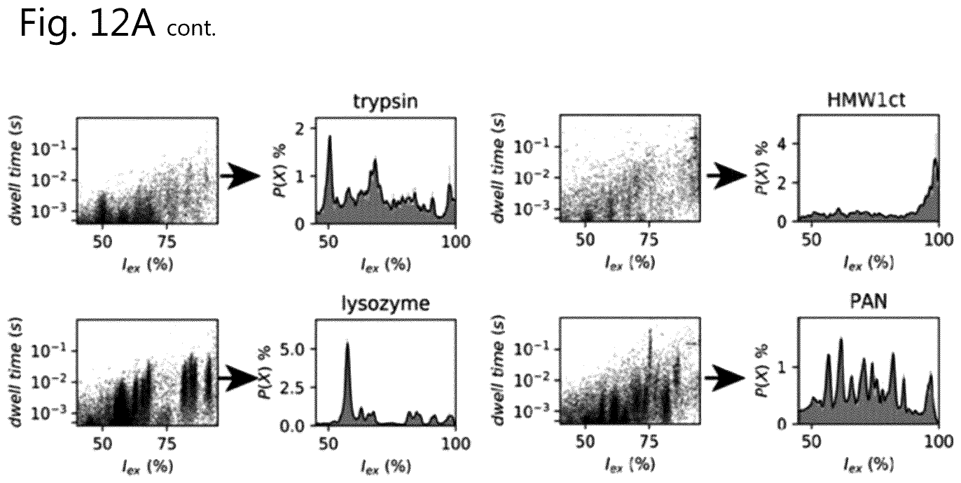

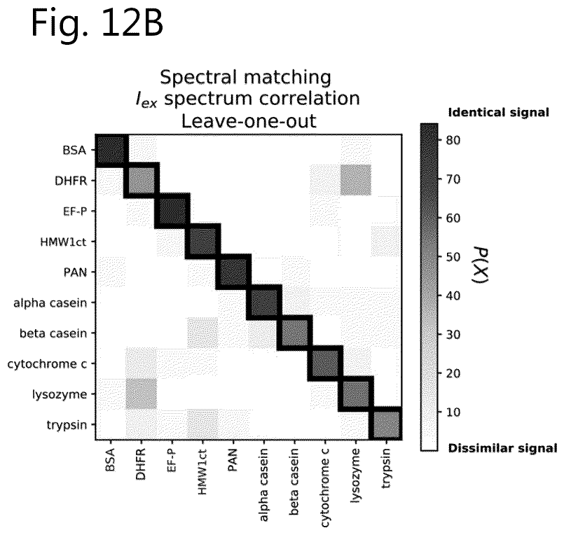

A further 9 proteins with highly divergent compositions were tested by nanopore spectrometry. The 9 proteins were: Bovine serum albumin (BSA), dihydrofolate reductase (DHFR), high molecular weight adhesin 1 (HMIW1Act), PAN unfoldase, Thiamine binding protein (TbpA), beta casein, cytochrome C, lysozyme and trypsin. The proteins were digested via trypsination as described. The resulting peptide fragment mixtures were separately tested in multiple separate nanopore experiments (G13F-FraC-T1 pores, 1M KCl, pH 3.8, −50 mV). Similar to what was observed for the digested lysozyme peptide mixture, distinct clusters of blockade events were observed from the peptide mixtures for each of the digested proteins (FIG. 11 and FIG. 12), with the clusters of event blockades separated by their excluded current Iₑₓ%. FIG. 11 shows that a high level of consistency for each unique spectra is observed between separate nanopore experiments for three representative protein samples.

To account for pore to pore variations in the baseline current, we aligned the residual current spectra to a reference spectrum using a sliding window on Iex %. FIG. 12 plots the aggregated histogram “excluded current spectra” from fits to the individual peptide blockade event scatter plots of excluded current versus dwell time for each protein sample. As would be expected, the excluded current spectra for each protein display unique patterns of peaks that are dependent on the unique composition of digested peptides in each system (with fragments varying by mass, length, and amino acid composition). Interestingly, the spectra of PAN and BSA show distinct peptide clusters, despite the large amount of fragments predicted from the in-silico digestion. This indicates that even large (50 kDa) proteins yield distinct spectra that can enable fingerprinting of the precursor protein.

Protein fingerprinting and spectral matching. The unique excluded current spectra of the tryptic digests (FIG. 12A) can be used to fingerprint proteins. The most straightforward way of fingerprinting is spectral matching, wherein the measured spectra are compared to a previously measured database of known spectra. Different datasets showed a high level of reproducibility (e.g. see FIG. 11) after taking the baseline shift from pore-to-pore variation in separate repeat experiments into account.

The uniqueness and reproducibility of the spectra were determined using spectral correlation, utilizing the squared first derivate Euclidean cosine correlation (DEuc) (Equation 2).

$$DEuc = \frac{{( {\sum }_{i} Δ {A}_{1 , i} Δ {A}_{2 , i} )}^{2}}{{\sum }_{i} Δ {A}_{1 , i}^{2} {\sum }_{i} Δ {A}_{2 , i}^{2}} \qquad ( 2 )$$

With A₁ and A₂ containing the vectors of the excluded current spectra and each element (1) in the vector⁹.

In order to ensure a representative example for spectral matching, we performed a leave-one-out comparison, where the comparison database was built from all spectra, excluding the one that was matched. The probability P(X) % was calculated from the DEuc score relative to the sum of all the DEuc scores (FIG. 12B). It was noticed that 8 of the 9 tryptic digests are correctly assigned to the known protein (diagonal axis), except for DHFR, which is erroneously assigned to lysozyme. Visual inspection of the DHFR and lysozyme spectra (FIG. 12A) readily explained the erroneous assignment, as both digests share some peak similarities for excluded current. This analysis employs only one “metric” of the events, their excluded current. We find that further analysis of spectra using other metrics, for example the standard deviation of the noise in each event, show that clusters/peaks that cannot easily be separated with one metric dimension are often possible to separate by another metric dimension.

### Detecting Amino Acid Changes

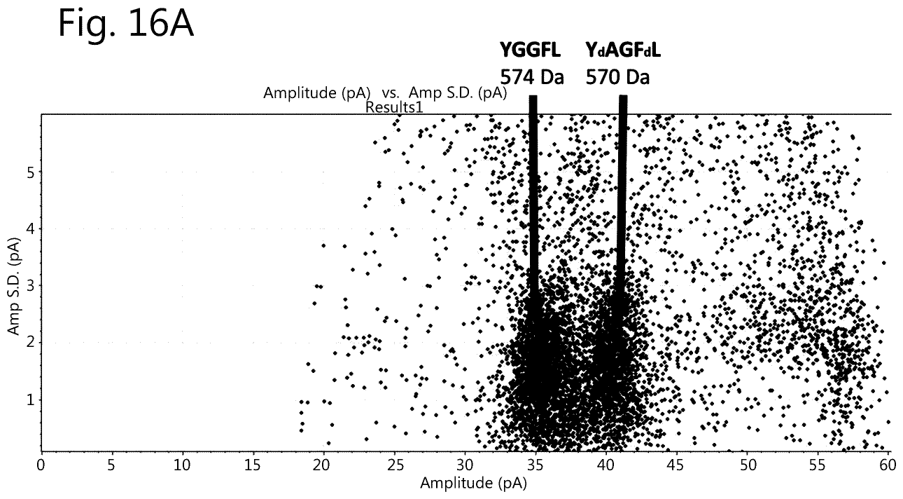

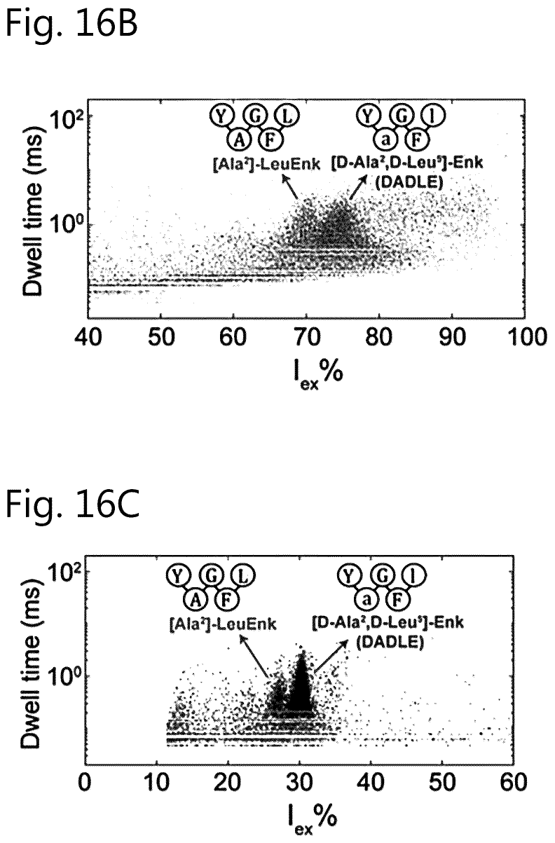

To evaluate the resolution of the analytical detection system to discriminate between peptides that differ by only 4 Dalton, two different forms of the enkephalin peptide were tested: YGGFL (SEQ ID NO: 36), and YdAGFdL (SEQ ID NO: 100), wherein d represents a D-amino acid; all other amino acids being in the L-configuration. FIG. 16A shows that two clear clusters are observed for the different peptides, illustrating that mass differences of at least 4 Da can be differentiated along with differences in chirality using exemplary FraC G13F nanopore. Detection of peptide chirality for peptides of the same mass was confirmed in FIGS. 16B and 16C, showing a difference in nanopore signal due to the presence of D-amino acids. A mixture 10 μM of [Ala2]-Leu Enkephalin and 10 μM DADLE ([D-Ala2, D-Leu5]-Enkephalin) was added to either the cis compartment (FraC-G13F; FIG. 16B) or trans compartment (CytK-K128F; FIG. 16C).

### Example 6: Detection of Post-Translationally Modified Peptides

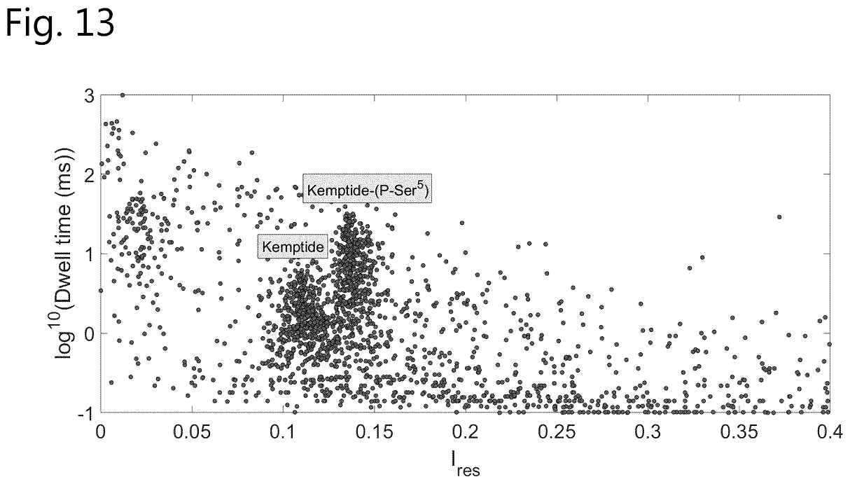

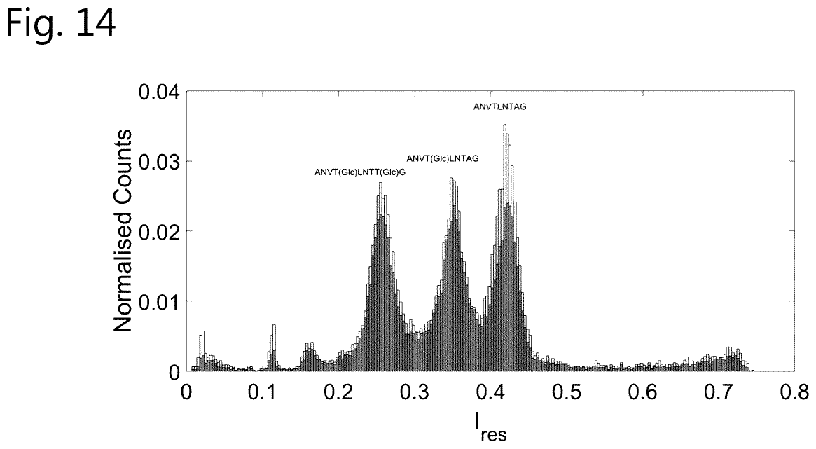

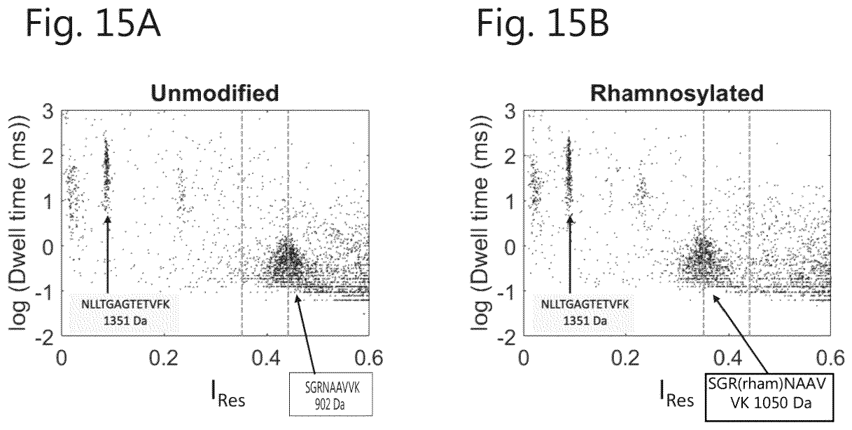

This example demonstrates that a mutant proteinaceous nanopore is capable of detecting post-translationally modified peptides. Aₙ analytical system comprising a FraC-G13F nanopore as described herein above was used to distinguish between a phosphorylated and non-phosphorylated peptide (see FIG. 13), an unmodified peptide, a peptide modified with a single or with two glycans (see FIG. 14), and unmodified protein and rhamnosylated protein (FIG. 15).

### Example 7: Mutant Proteinaceous Nanopore Comprising a Beta-Barrel Pore Forming Toxin

Examples 1 to 6 relate to a mutant proteinaceous nanopore comprising an alpha-helical pore-forming toxin of the actinoporin family, and its application as single molecule sensor. To test whether these discoveries were more broadly applicable to different classes of nanopores, with similar dimensions in the sensing region but quite different structural makeup, we explored similar mutations and conditions on beta-barrel pores.

This example demonstrates that beta-barrel pore-forming proteins wherein the lumen-facing recognition region of the proteins comprises one or more mutations to an aromatic residue can also be used to provide such nanopore-based sensors, particularly in combination with nearby acidic mutations.

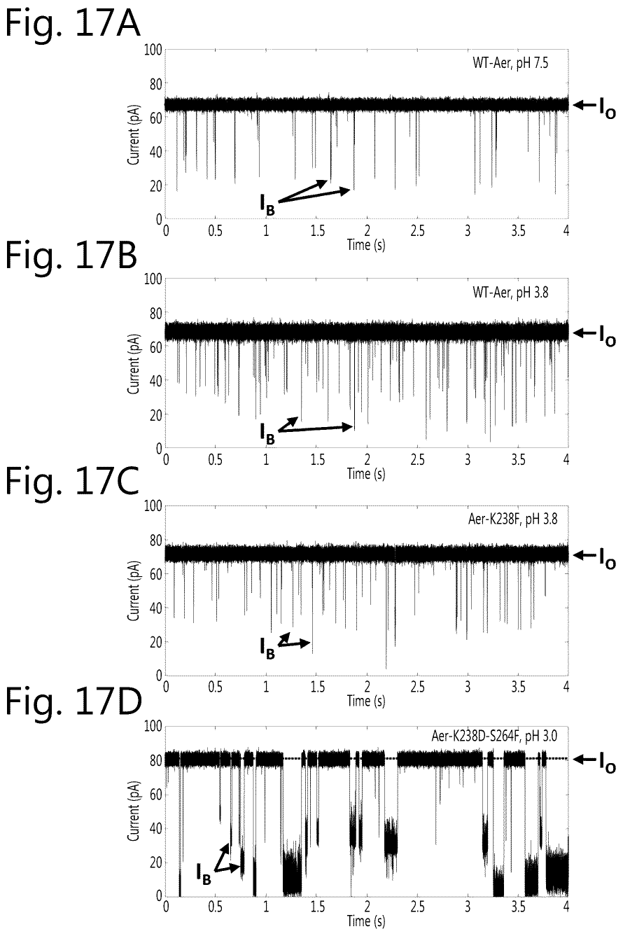

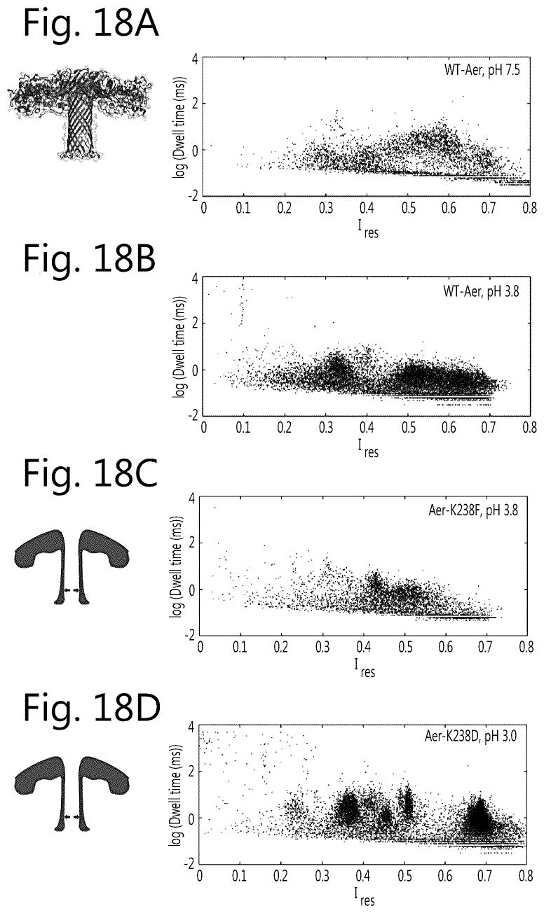

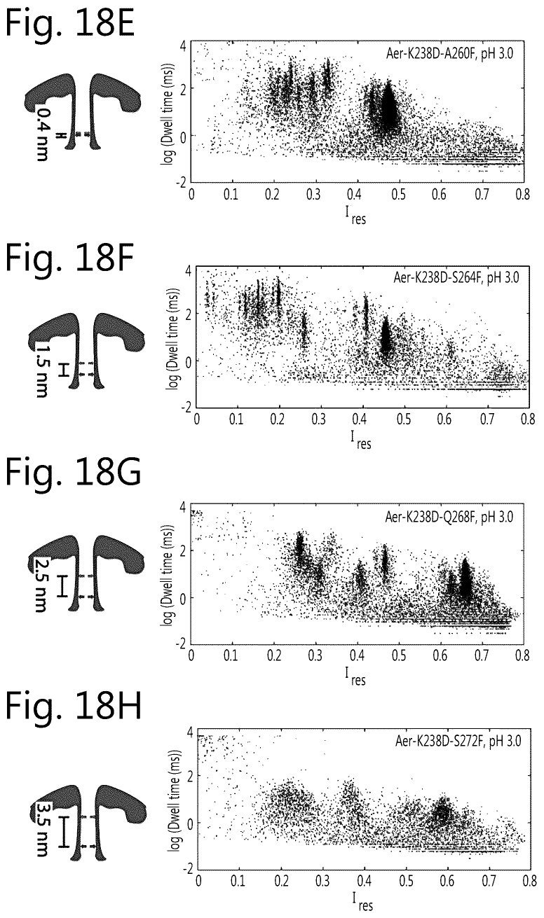

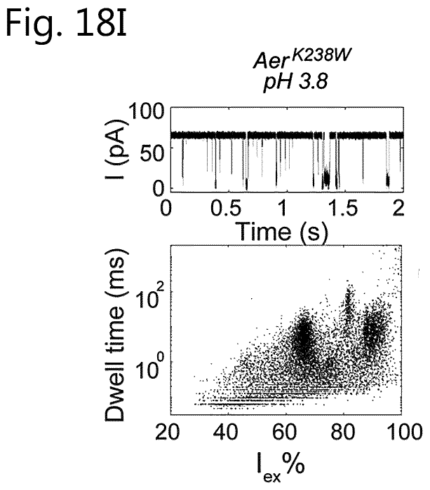

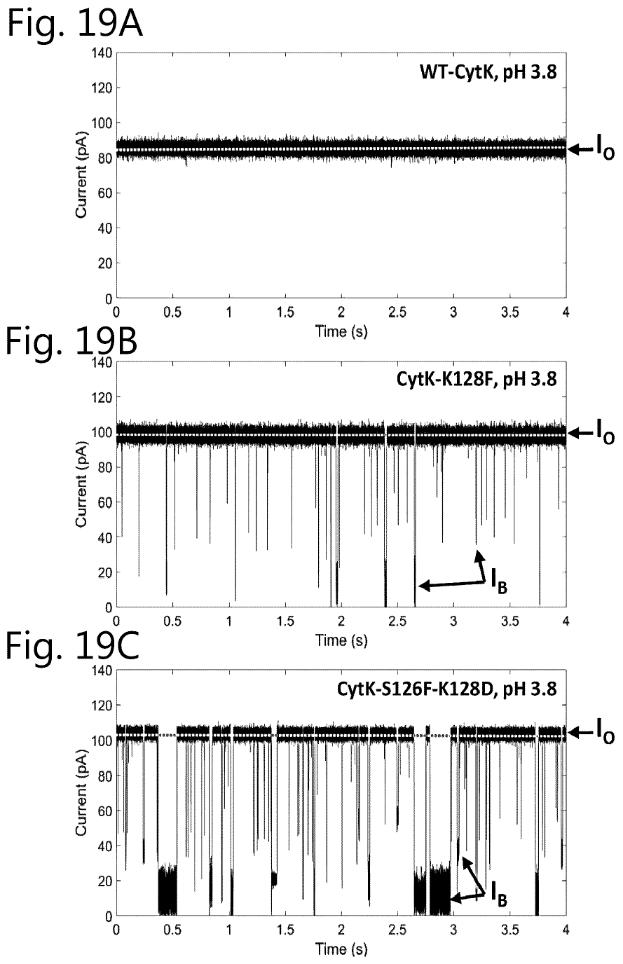

It was found that lowering the pH of the buffer could increase the capture (FIG. 17A versus FIG. 17B) and resolution (FIG. 18A versus FIG. 18B) of a tryptic digested peptide mixture using the wild-type Aerolysin pore. However, for the wild-type pore, even at low pH (e.g. pH 3.8) the events that we observe are extremely short (FIG. 17A/B) and peptide clusters resulting from different peptide populations have a wide distribution and are poorly resolved from each other, making the distinction of individual peptides from the mixture challenging (FIG. 18B).

We found that replacing the Lysine at position 238 with a phenylalanine (Aer-K238F, FIGS. 17C and 18C) did not significantly increase the dwell time of peptides (FIG. 17C) and only marginally improved peptide cluster resolution under pH 3.8 (FIG. 18C), and that replacing the Lysine residue at position 238 by the acidic amino acid aspartate (Aer-K238D) significantly increased the cluster resolution at low pH (FIG. 18D) over the wild-type pore. The improved peptide capture and resolution for the K238D mutation is partly due to reduced electrostatic repulsion between the recognition region of the nanopore and the mostly positively charged peptides at low pH, and partly due to the increased cation ion-selectivity.

We further combined the K238D mutation with the introduction of the phenylalanine at either position Ala260, Ser264, Gln268 or S272 of Aerolysin, and observed a dramatic improvement in peptide resolution (FIG. 18E-H). The improved resolution between different peptide clusters is the result of a combination of improvements, including 1) longer residence (dwell) times that enable more accurate measurement of each single-molecule event (e.g. FIG. 17D), 2) a lower spread of residual currents in each cluster that enables closely separated clusters to be resolved from one another more easily, and 3) clusters spread out more widely over the full current range. The resolution of the analyte peptides was especially sharp when the distance between the aspartic acidic at position 238 and the introduced aromatic amino acid was less than 4 nm. Therefore, the combination of an increased negative pore and an aromatic substitution on the water-facing transmembrane is important for increasing the capture and resolution of unlabeled peptides. This appears especially important when sampling at acidic pH values (<pH 4.5). Importantly, this combination of mutations in the lumen of the beta-barrel pore creates similar rings of sensing residues to those in the constriction of the FraC nanopore when engineered for improved peptide discrimination, showing that this combination of mutations is a general feature that can be engineered into the sensing constriction of a wide range of both alpha-helical and beta-barrel based nanopores with similar sensing constriction geometries (for example, engineering mutations into non-conserved inward facing residues through the use of a combination of well-known structural and homology modelling tools known in the art).

FIG. 181 shows the capture and resolution of a tryptic digested peptide mixture using the mutant Aer-K238W pore, and demonstrates that the aromatic mutation significantly improves peptide detection versus the wild-type aerolysin.

### Expression and Purification of Pro-Aerolysin

Plasmid containing a gene encoding for pro-aerolysin elongated by a hexa-histidine tag (SEQ ID NO: 101) at the C-terminus was transformed into BL21(DE3) cells using electroporation. The transformed cells were grown overnight at 37° C. on LB agar plates supplemented with 1% glucose and 100 μg/ml ampicillin. On the next day, the colonies are resuspended and grown in 200 mL 2YT medium at 37° C. until the OD₆₀₀ reached 0.6-0.8. At this point, the expression was induced by addition of 0.5 mM IPTG and the culture was incubated overnight at 25° C. Afterwards, the cells were pelleted by centrifugation at 4000 rpm for 15 minutes and the cell pellets were stored at −80° C. for at least 30 minutes. For protein purification, cell pellets of 100 ml culture were resuspended in 20 ml lysis buffer, containing 150 mM NaCl, 20 mM imidazole and 15 mM Tris buffered to pH 7.5, supplemented with 1 mM MgCl₂, 0.2 units/ml DNase1 and approximately 1 mg of lysozyme. The mixture is incubated for 30 minutes at RT and afterwards sonicated using a Branson Sonifier 450 (2 minutes, duty cycle 30%, output control 3) to ensure full disruption of the cells. Cell debris is pelleted by centrifugation at 6000 rpm for 20 minutes and the supernatant is carefully transferred to a fresh falcon tube. Meanwhile, 200 μl of Ni-NTA bead solution is washed with wash buffer, containing 150 mM NaCl, 20 mM imidazole and 15 mM Tris buffered to pH 7.5. The beads are added to the supernatant and incubated at RT for 5 minutes. Afterwards, the solution is loaded on a Micro Bio-Spin column (Bio-Rad) and subsequently washed with 5 ml of wash buffer. The bound protein is eluted in fractions of 200 μl of elution buffer (150 mM NaCl, 300 mM imidazole, 15 mM Tris buffered at pH 7.5. The pro-aerolysin fractions can be stored in at 4° C. for several weeks.

Oligomerisation from Pro-Aerolysin Using Trypsin

Pro-aerolysin is incubated with trypsin in a 1:1000 mass ratio for 15 minutes at room temperature. The trypsin cleaves off the C-terminal peptide, resulting in aerolysin monomers that can assemble into heptameric pores, which pores can be characterised in electrophysiology experiments.

### Planar Lipid Bilayer Electrophysiological Recordings.

The electrophysiology chamber consisted of two compartments separated by a 25 μm thick Teflon (Goodfellow Cambridge Ltd) membrane. The Teflon membrane contained an aperture with a diameter of approximately 100-200 μm. Lipid membranes were formed by first applying 5 μl of 5% hexadecane (Sigma Aldrich) in pentane (Sigma Aldrich) to the Teflon membrane, near the aperture. The pentane was left to dry and 400 μl of run buffer (1 M KCl, 50 mM citric acid, titrated with bis-tris propane to pH 3.8 or pH 3.0; or 1 M KCl, 50 mM Tris buffered at pH 7.5) was added to both sides. 20 μl of a 10 mg/ml solution of DPhPC dissolved in pentane was added on top of the buffer on each side of the chamber. The chamber was left to dry for −2 minutes to allow evaporation of pentane. Silver/silver chloride electrodes were attached to each compartment. The cis compartment was connected to the ground electrode and the trans was connected to the working electrode. Planar lipid bilayers were created using the Langmuir-Blodgett method described by Maglia et al (Huang et al. Nat. Commun. 2017).

### Tryptic Digestion of Lysozyme

100 μg of lysozyme (Carl Roth, from chicken egg white, albumin free) was dissolved in 150 mM NaCl, 15 mM Tris buffered at pH 7.5. Before digestion, free cysteines were alkylated to prevent formation of disulfide bridges after digestion. To that end, 3 μL 200 mM DTT was added and the sample was incubated at 37° C. for 15 min, followed by 15 minutes of denaturation at 95° C. Subsequently, 7 μL of 200 mM IAA was added and the sample was incubated for 15 min at RT in the dark. After alkylation, the lysozyme was digested overnight at 37° C. in a 50:1 lysozyme:trypsin mass ratio using the Trypsin Singles, Proteomics Grade-kit (Sigma Aldrich, Catalog #T7575-1KT).

### Detection of Lysozyme Digest Using Aerolysin Pores

Aerolysin was added to the cis-chamber and the bilayer was broken and reformed until a single channel inserted into the bilayer. The orientation of the pore can be detected by a small asymmetry in the IV curve of the pore. First, a 2 minute blank was recorded at +150 mV applied potential and afterwards 4 μl of trypsin-digested lysozyme was added to the cis compartment of the chamber. The analyte was measured for at least 10 minutes at an applied potential of +150 mV.

Data recording. Recordings of ionic currents were obtained using an Axopatch 200B (Axon Instruments) combined with a Digidata 1550B A/D converter (Axon instruments), similar to preceding work (Huang et al. Nat. Commun. 2019). The sampling frequency was set at 50 kHz for analyte recordings, the analogue Bessel filter was set at 10 kHz. Data was recorded using Clampex 10 (Molecular Devices).

### Example 8. Mutant Proteinaceous Nanopore Comprising a Cytolysin k Beta-Barrel Pore Forming Protein

Example 7 relates to single molecule analysis using a modified beta-barrel pore-forming protein Aerolysin. In this example, functionally similar mutations were introduced into the cytolysin k (cytK) nanopore to demonstrate that aromatic mutations, preferably in combination with nearby acidic mutations, preferably when used under low pH conditions (<pH 4), improve the ability to capture and resolve unlabelled peptides for other beta-barrel pores.

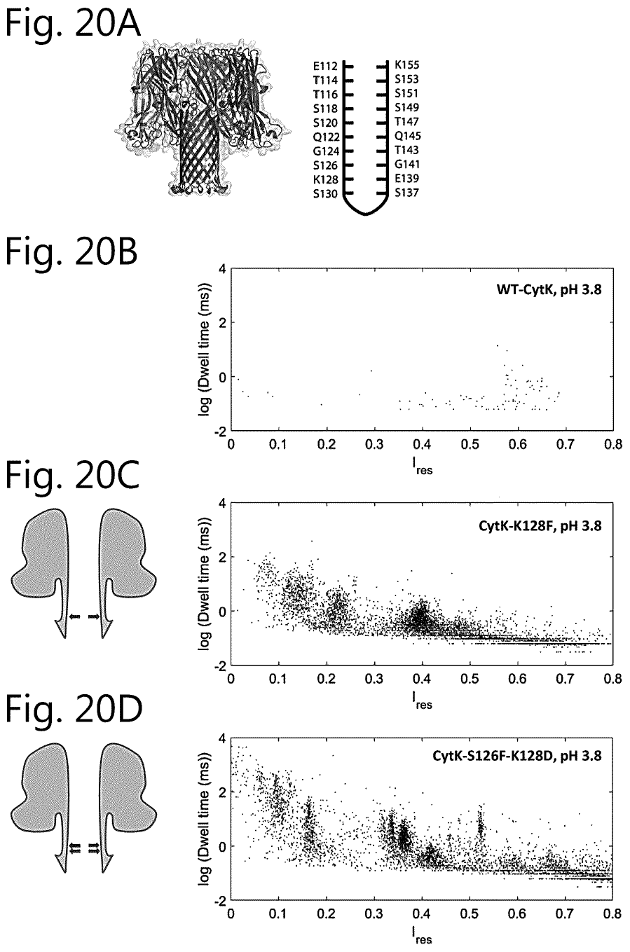

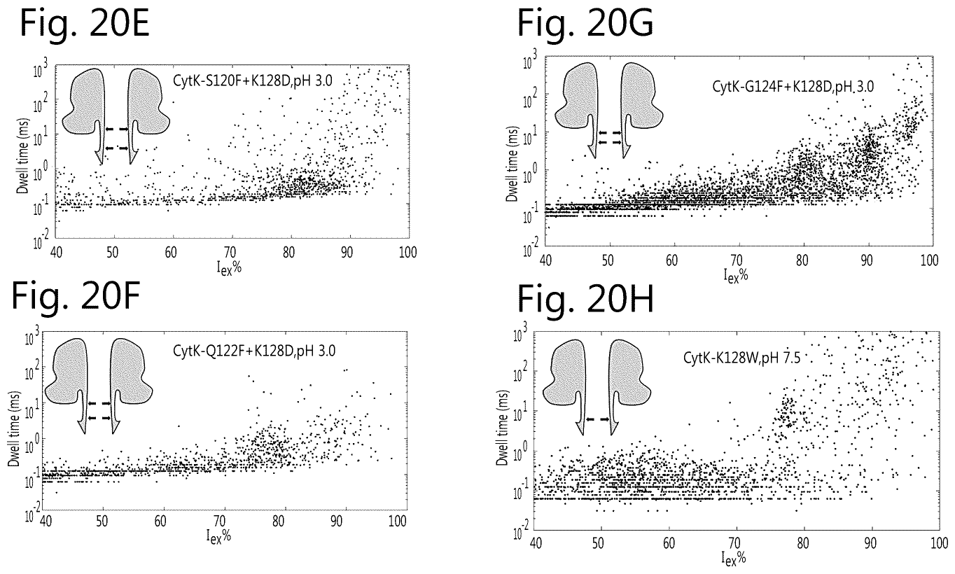

Although CytK is known to be a nanopore capable of passing current when inserted into a membrane (Hardy et al, FEMS Microbiol Lett. 2001), the structure of CytK is not known. Therefore, to identify the beta-barrel region, and the putative analyte recognition region, a homology model was built by mapping the CytK sequence to the sequence and structure of the alpha-hemolysin nanopore from Staphylococcus aureus (FIG. 20A). We identified the beta-barrel region as comprising the stretch running from amino acid E112 to amino acid 5134, and from amino acid 5137 to amino acid K155, with the even residues in the range E112-S130 and odd residues in the range 5137-K155 being the inward lumen water-facing residues (FIG. 20A).

### Expression and Purification of (Mutant) CytK

Plasmid containing a gene encoding for CytK elongated by six histidine residues (SEQ ID NO: 101) at the C-terminus was transformed into BL21(DE3) cells by electroporation. Transformed cells were grown overnight at 37° C. on LB agar plates (1% glucose, 100 μg/ml ampicillin). Colonies were resuspended and grown in 200 mL 2YT medium at 37° C. until OD₆₀₀ 0.6-0.8, then expression was induced by addition of 0.5 mM IPTG and the culture was incubated overnight at 25° C. Cells were pelleted by centrifugation and stored at −80° C. for at least 30 minutes. Cell pellets were lysed by resuspension in lysis buffer (150 mM NaCl, 20 mM imidazole, 15 mM Tris pH 7.5, 1 mM MgCl₂, 0.2 units/ml DNase1, ˜1 mg of lysozyme), incubated for 30 minutes at RT, then sonicated (Branson Sonifier 450, 2 minutes). Cellular debris was pelleted by centrifugation and the supernatant containing CytK was recovered. CytK was extracted from the supernatant and purified using Ni-NTA beads, with final elution in 200 μl aliquots (150 mM NaCl, 300 mM imidazole, 15 mM Tris buffered at pH 7.5) before storage at 4° C.

### Planar Lipid Bilayer Electrophysiological Recordings.

Electrophysiology measurements were performed as described in Example 7. CytK was added to the cis-chamber and the DPhPC bilayer in the nanopore system was broken and reformed until a single nanopore inserted into the bilayer. The orientation of the pore can be detected by the asymmetry in the IV curve of the pore. All recordings were performed with 1 M KCl in both the cis and trans compartments at either pH 3.8 (50 mM citric acid, titrated with bis-tris propane to pH 3.8) or pH 7.5 (50 mM Tris buffered at pH 7.5). First, 2 minutes of blank open-pore current was recorded at +100 mV applied potential, and afterwards 4 μl of trypsin-digested lysozyme was added to either this cis or trans compartment of the chamber. The analyte was measured for at least 10 minutes at an applied potentials of −100 mV to +100 mV as indicated. The ionic current was recorded using a Digidata 1440A (Molecular Devices) connected to an Axopatch 200B amplifier (Molecular Devices). The sampling frequency was set at 50 kHz for analyte recordings, the analogue Bessel filter was set at 10 kHz. Data was recorded using Clampex 10 (Molecular Devices). Event blockade data was analysed as described herein, measuring the event blockades resulting from peptide capture and extracting metrics including average open-pore current, average blockade current, blockade duration (dwell time), standard deviation of blockade current, etc.

### Results

Similar to Example 7, nanopore sensing systems containing CytK nanopores were tested using a digested peptide mixture resulting from trypsinated lysozyme. Wild Type CytK exhibits little to no capture of the peptides from a trypsinated lysozyme sample, including when the sample is added to either the cis or trans compartments, under either positive or negative applied potentials over a wide range of voltages, at either pH 7.5 or pH 3.8. For example, FIG. 19A and FIG. 20B shows the low number of detected events using wildtype CytK nanopores when trypsinated lysozyme sample was added to the trans compartment, with a positive applied potential at the trans electrode to drive electrophoretic capture of the mostly positively charged peptides (+100 mV, 1M KCl, pH 3.8).

According to our predicted structure, a Lysine residue at position 128 and a Glutamate residue at position 139 are predicted to be inward facing residues in the recognition region. In accordance with previous findings described herein, a phenylalanine was substituted into the K128 position of the CytK monomers adjacent to the acidic E139, thus serving both to reduce the net positive charge in the nanopore and introduce an aromatic for improved peptide detection. The K128F mutation produced a dramatic improvement in the ability to both capture (FIG. 19B) and resolve (FIG. 20C) different peptides at low pH versus the wild-type nanopore. Very good results were also obtained with the K128W mutation (FIG. 20H).

In another implementation, similar to the strategy employed in Example 7, an aromatic amino acid was introduced adjacent to an additional negative mutation by substituting the lysine at 238 with an aspartic acid and substituting the serine at 126 with a phenylalanine (CytK-S126F-K128D). Similar to what was observed for the Aerolysin nanopore system, this combination of an aromatic amino acid substitution adjacent to an acidic amino acid substitution further improved the resolution of different peptides through a combination of improved metrics, including: better capture (FIG. 19C), longer residence (dwell time) of peptide blockades (FIG. 19C), tighter clusters with less residual current spread (FIG. 20D), and clusters spread widely over the full min-max current range (FIG. 20D).

Aromatic mutations placed higher up in the barrel of aerolysin (position S120, Q122 or G124) combined with K128D also yielded a good resolution of peptides of a trypsinated lysozyme sample. See FIGS. 20E, F and G.

Accordingly, the data demonstrates that aromatic mutations, preferably adjacent to acidic amino-acid substitutions, creates a sensing region that improves the ability to capture and discriminate unlabeled peptides, in particular at low pH conditions.

Notably, in example 7 and 8 we have demonstrated two different dominant mechanisms for controlling peptide capture in CytK and aerolysin nanopores. For example, we demonstrated that Aerolysin nanopores can capture and discriminate peptides effectively at positive applied potential when analytes are in the cis compartment. Therefore, the analytes, being mostly positively charged at pH 3.8 or pH 3.0, are captured against the electrophoretic direction due to dominant electro-osmotic capture conditions. In contrast, we demonstrated that CytK can capture and discriminate peptides effectively at positive applied potential when analytes are in the trans compartment. Therefore, the analytes are captured primarily by electrophoretic forces under the pH 3.8 conditions, and the electro-osmotic component was tuned to be close to zero by substitution of additional acidic residues (see Table 4). Our results indicate that the introduction of aromatic residues in beta-barrel pore-forming toxins works regardless of the capture mechanism of the analyte, and that the introduction of acidic residues under low pH conditions is an important tool for tuning and controlling cation selectivity and electro-osmotic capture.

TABLE 4
Ion selectivity of FraC, Aerolysin and CytK nanopores. The reversal
potential was measured from IV curves between −100 mV
and +100 mV under asymmetric salt conditions (2M KCl in trans
and 0.5 M KCl in cis), buffered to indicated pH using 50 mM
Tris for pH 7.5 or 50 mM citric acid titrated to pH 3.8 using
bis-tris propane. The reversal potential (the applied voltage
at which there is zero net current flow) was determined by
linear regression of the IV curve between −20 mV and +20 mV.

| | | Reversal potential | |
|---|---|---|---|
| | | (mV) | P(K⁺)/P(Cl⁻) |
| Wt Aerolysin | pH 7.5 | −3.5 ± 0.4 | 0.78 |
| | pH 3.8 | −13.2 ± 0.4 | 0.37 |
| Aerolysin K238D | pH 3.8 | −8.8 ± 0.9 | 0.52 |
| Aerolysin_K238W | pH 3.8 | −10.0 ± 0.8 | 0.48 |
| Wt CytK | pH 7.5 | −0.3 ± 0.3 | 0.98 |
| | pH 3.8 | −7.8 ± 0.8 | 0.57 |
| CytK_K128F | pH 7.5 | 12.8 ± 0.2 | 2.61 |
| | pH 3.8 | 0.5 ± 0.3 | 1.03 |
| CytK_128D | pH 7.5 | 10.5 ± 0.2 | 2.17 |
| | pH 3.8 | 1.7 ± 0.7 | 1.12 |
| WtFraC* | pH 7.5 | 17.2 ± 1.2 | 3.6 ± 0.4 |
| | pH 3.8 | 1.0 ± 1.7 | 1.03 ± 0.04 |
| FraC_G13F | pH 7.5 | 17.0 ± 0.7 | 3.7 ± 0.2 |
| | pH 3.8 | 0.0 ± 0.4 | 1.00 ± 0.03 |

*replicated from Huang et al., Nat. Commun. 2019, 10 (1), 835.

### Example 9. Mutant Proteinaceous Nanopore Comprising a Lysenin Beta-Barrel Pore Forming Protein

This example shows that Lysenin, a further exemplary beta-barrel pore forming protein, is successfully mutated to demonstrate that an aromatic mutation of a non-aromatic lumen facing residue improves the ability to capture and resolve unlabelled peptides.

Plasmids containing the Lysenin gene from Eiseniafetida were transformed into BL21(DE3) E. coli competent cell by electroporation. Next, the cells were grown on lysogeny broth (LB) agar plate containing 100 μL/mL ampicillin overnight at 37° C. The LB plate was harvested and inoculated into 400 mL 2×YT media. Then, the culture was grown at 37° C. while shaking at 200 rpm until the optical density at 600 nm of the cell culture reached 0.8. This was followed by addition of 0.5 mM isopropyl-D-thiogalactoside (IPTG) to the media and the culture was grown overnight at 25° C. while shaking at 200 rpm. The next day, cells were harvested by centrifugation (4000 rpm, 15 min) and the resulting pellets were frozen at −80° C. for 30 min.

The cells were resuspended and mixed for 30 min in 40 mL of lysis buffer (50 mM Tris-HCl (pH 7.5), 150 mM NaCl and 0.02% DDM supplemented with, 10 mM imidazole, 1 mM MgCl₂) together with 0.2 mg/mL lysozyme, and 10 μL DNaseI. The lysate was sonicated for 2 min (40% output power) and centrifuged down at 4° C. for 15 min (4000 rpm). Next, the supernatant was incubated with 150 μL washed Ni-NTA beads for 15 min at 20 rpm. The Ni-NTA beads were loaded on a gravity-flow column and washed with wash* buffer: ([50 mM Tris-HCl (pH 7.5), 150 mM NaCl, 10 mM imidazole, and 0.02% DDM)]. The proteins were eluted in 3 elution steps with 150 μL elution buffer:* ([50 mM Tris-HCl (pH 7.5), 150 mM NaCl, 300 mM imidazole and 0.02% DDM)]. Lysenin monomers were stored at 4° C.

Lysenin can be oligomerized by incubation with liposomes (with a 1:1 sphingomyelin:DPHPC lipid composition) in a 1:10 protein:liposome ratio at 37° C. for 1 hour. The liposomes are then disrupted by addition of 0.6% LDAO. The solution is diluted 20× using wash buffer and mixed with 150 μl washed Ni-NTA beads. The solution is subsequently loaded on a gravity-flow column and washed with wash buffer. Oligomers are eluted by an elution buffer containing 1M Imidazole, 150 mM NaCl and 15 mM Tris buffered to pH 7.5 in fractions of 150 μl. Oligomers were stored at 4° C.

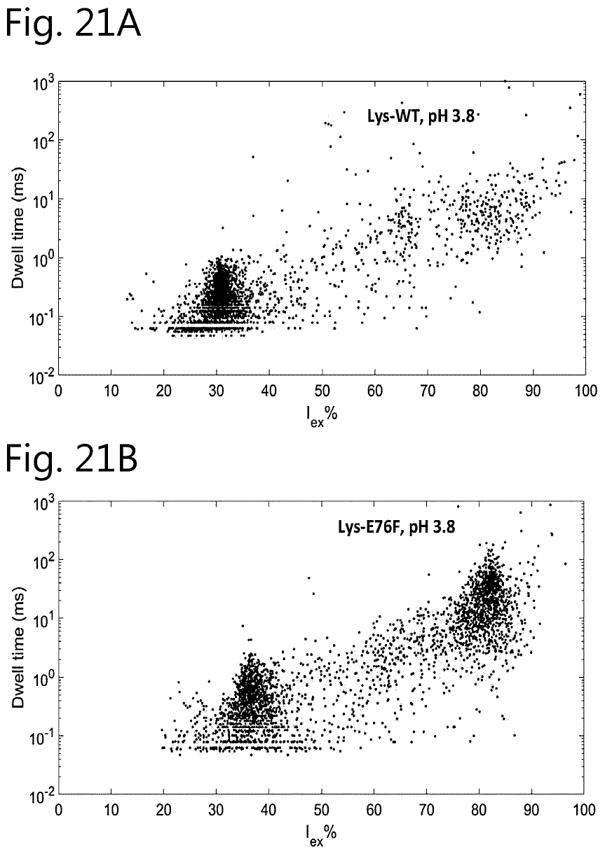

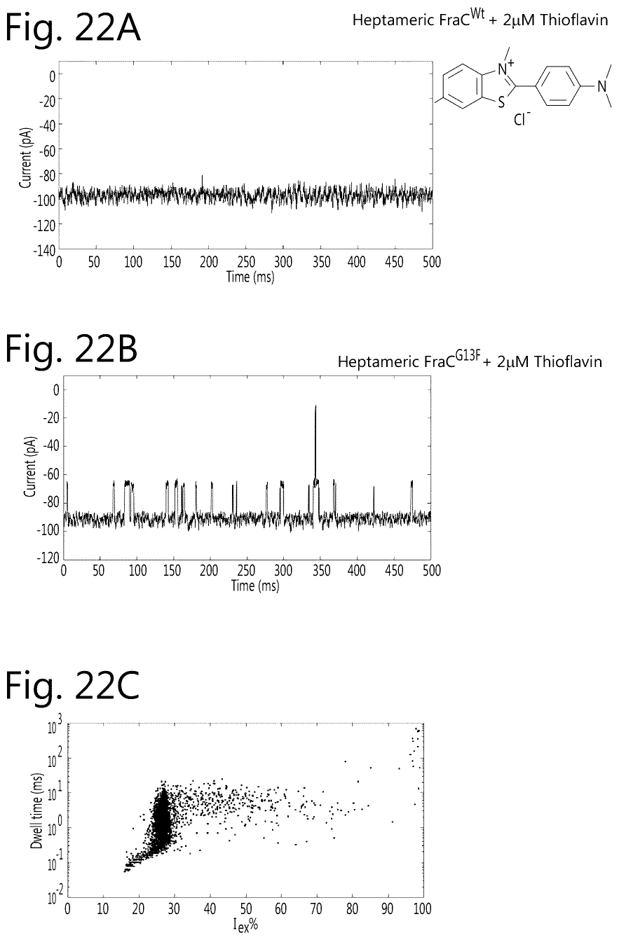

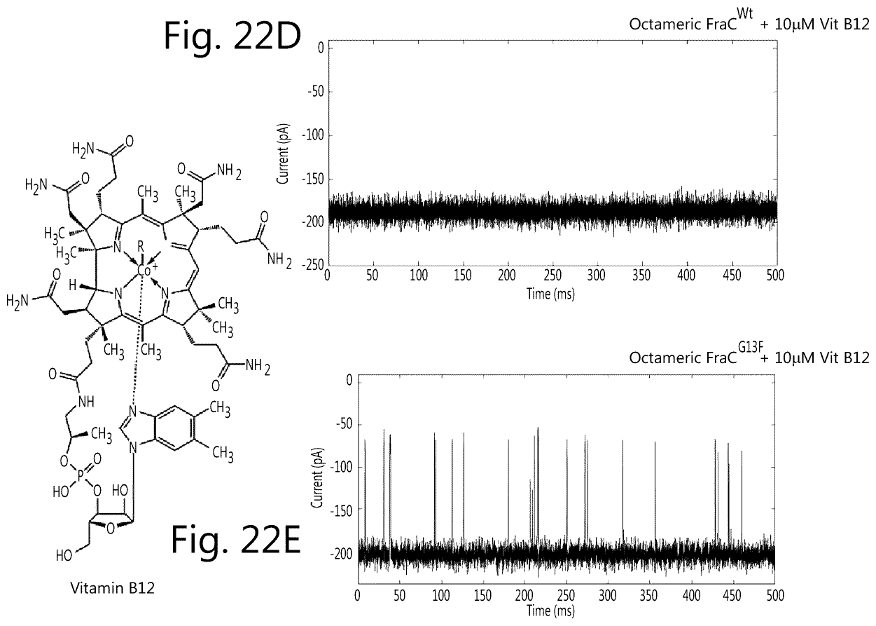

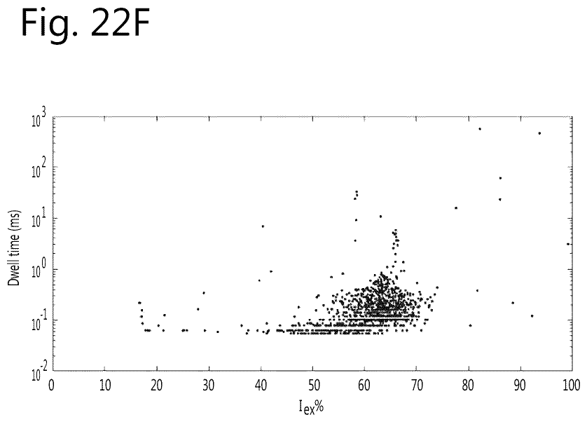

FIG. 21 shows the results obtained with 0.5 μg Lys-C digested lysozyme added to the trans compartment (final concentration 1.25 ng/μl) of an analytical system comprising either wildtype Lys (panel A) or Lys-E76F (panel B). Introduction of the aromatic residue in the lumen results in a clear peptide cluster for larger peptides.

### REFERENCES

(1) Robertson, J. W. F.; Rodrigues, C. G.; Stanford, V. M.; Rubinson, K. A.; Krasilnikov, O. V.; Kasianowicz, J. J. Single-Molecule Mass Spectrometry in Solution Using a Solitary Nanopore. Proc. Natl. Acad. Sci. 2007, 104 (20), 8207-8211.
(2) Huang, G.; Voet, A.; Maglia, G. FraC Nanopores with Adjustable Diameter Identify the Mass of Opposite-Charge Peptides with 44 Dalton Resolution. Nat. Commun. 2019, 10 (1), 835.
(3) Chavis, A. E.; Brady, K. T.; Hatmaker, G. A.; Angevine, C. E.; Kothalawala, N.; Dass, A.; Robertson, J. W. F.; Reiner, J. E. Single Molecule Nanopore Spectrometry for Peptide Detection. ACS Sensors 2017, 2 (9), 1319-1328.
(4) Anderluh, G.; Macek, P. Cytolytic Peptide and Protein Toxins from Sea Anemones (Anthozoa: Actiniaria). Toxicon 2002, 40 (2), 111-124.
(5) Garcia-Ortega, L.; Alegre-Cebollada, J.; Garcia-Linares, S.; Bruix, M.; Martinez-del-Pozo, Á.; Gavilanes, J. G. The Behavior of Sea Anemone Actinoporins at the Water-Membrane Interface. Biochim. Biophys. Acta—Biomembr. 2011, 1808 (9), 2275-2288.
(6) Ros, U.; Rodriguez-Vera, W.; Pedrera, L.; Valiente, P. A.; Cabezas, S.; Lanio, M. E.; Garcia-Sáez, A. J.; Alvarez, C. Differences in Activity of Actinoporins Are Related with the Hydrophobicity of Their N-Terminus. Biochimie 2015, 116, 70-78.
(7) Huang, G.; Willems, K.; Soskine, M.; Wloka, C.; Maglia, G. Electro-Osmotic Capture and Ionic Discrimination of Peptide and Protein Biomarkers with FraC Nanopores. Nat. Commun. 2017, 8 (1), 935.
(8) Tanaka, K.; Caaveiro, J. M. M.; Morante, K.; González-Manas, J. M.; Tsumoto, K. Structural Basis for Self-Assembly of a Cytolytic Pore Lined by Protein and Lipid. Nat Commun 2015, 6.
(9) Li, J.; Hibbert, D. B.; Fuller, S.; Vaughn, G. A Comparative Study of Point-to-Point Algorithms for Matching Spectra. Chemom. Intell. Lab. Syst. 2006, 82 (1), 50-58.
(10) Müllner, D. Fastcluster: Fast Hierarchical, Agglomerative Clustering Routines for R and Python. J. Stat. Software; Vol 1, Issue 9 2013.
(11) Thapa, P et al.; Native chemical ligation: a boon to peptide chemistry. Molecules. 2014 Sep. 12; 19(9):14461-83.
(12) Hardy, S. P, Granum, E., CytK toxin of Bacillus cereus forms pores in planar lipid bilayers and is cytotoxic to intestinal epithelia. FEMS Microbiol Lett. 2001; 197(1):47-51
(13) Dal Peraro, van der Goot, Pore-forming toxins: ancient, but never really out of fashion. Nat Rev Microbiol. 2016 February; 14(2):77-92.
(14) Shengli Zhang, Gang Huang, Roderick Versloot, Bart Marlon Herwig, Paulo Cesar Telles de Souza, Siewert-Jan Marrink, Giovanni Maglia, Bottom-up fabrication of a multi-component nanopore sensor that unfolds, processes and recognizes single proteins. BioRxiv, 2020.
(15) Scott et al. Constructing ion channels from water-soluble α-helical barrels. Nat Chem. 2021 May 10.
(16) Vorobieva et al. De novo design of transmembrane β barrels. Science. 2021 Feb. 19; 371(6531)
(17) Spruijt, Tusk, Bayley. DNA scaffolds support stable and uniform peptide nanopores. Nat Nanotechnol. 2018 August; 13(8):739-745.
(18) Kristan, K. C., Viero, G., Dalla Serra, M., Macek, P. & Anderluh, G. Molecular mechanism of pore formation by actinoporins. Toxicon, 2009 1125-1134.
(19) Heron et al, Simultaneous measurement of ionic current and fluorescence from single protein pores. J Am Chem Soc. 2009; 131(5):1652-3.
(20) Spaan, van Strijp, Torres, Leukocidins: staphylococcal bi-component pore-forming toxins find their receptors. Nat Rev Microbiol. 2017 July; 15(7):435-447.
(21) Hammerstein, Jayasinghe, Bayley. Subunit dimers of alpha-hemolysin expand the engineering toolbox for protein nanopores. J Biol Chem. 2011 Apr. 22; 286(16):14324-34.
(22) Gouaux et al. Subunit stoichiometry of staphylococcal alpha-hemolysin in crystals and on membranes: a heptameric transmembrane pore. Proc Natl Acad Sci USA. 1994 Dec. 20; 91(26):12828-31.
(23) Crnković, Srnko, Anderluh. Biological Nanopores: Engineering on Demand. Life (Basel). 2021 Jan. 5; 11(1):27.

1-23. (canceled)

24. A nanopore, comprising:
(i) at least a portion of an alpha helical pore forming protein or peptide, or (ii) at least a portion of a beta barrel pore forming protein or peptide, wherein the at least the portion of the beta barrel pore forming protein or peptide is not an alpha-hemolysin or is not an aerolysin, wherein the at least the portion of the alpha helical pore forming protein or peptide or the at least the portion of the beta barrel pore forming protein or peptide comprises a modification of one or more lumen facing amino acids into one or more natural or non-natural aromatic amino acids.

25. The nanopore of claim 24, wherein the nanopore comprises the at least the portion of the alpha helical pore forming protein or peptide.

26. The nanopore of claim 24, wherein the at least the portion of the alpha helical pore forming protein or peptide comprises an actinoporin.

27. The nanopore of claim 24, wherein the nanopore comprises the at least the portion of the beta barrel pore forming protein or peptide.

28. The nanopore of claim 24, wherein the at least the portion of the beta barrel pore forming protein or peptide comprises a cytolysin, leukocidin, bacterial outer membrane porin, or a de novo designed pore peptide.

29. The nanopore of claim 24, wherein the modification of the one or more lumen facing amino acids is within or adjacent to a constriction region of the nanopore.

30. The nanopore of claim 29, wherein the constriction region of the nanopore comprises an internal diameter of at most about 2 nanometers.

31. The nanopore of claim 24, wherein the one or more lumen facing amino acids comprises an amino acid that is in contact with a central channel of the nanopore.

32. The nanopore of claim 24, wherein the modification comprises an amino acid sequence insertion, an amino acid sequence substitution, a chemical modification, a chemical ligation, a chemical functionalization, or a combination thereof.

33. The nanopore of claim 24, wherein the nanopore comprises an increase in aromatic rings in the lumen of the nanopore, as compared to another nanopore without the modification.

34. The nanopore of claim 24, wherein the alpha helical pore forming protein comprises an amino acid sequence that is at least 80% identical to an amino acid sequence of Fragaceatoxin C.

35. The nanopore of claim 24, wherein the at least the portion of the beta barrel pore forming protein or peptide comprises a Cytolysin K, lysenin, Anthrax toxin, Mycobacterium smegmatis porin A (MspA), Mycobacterium smegmatis porin B (MspB), Mycobacterium smegmatis porin C (MspC), Mycobacterium smegmatis porin D (MspD), outer membrane porin F (OmpF), outer membrane porin G (OmpG), outer membrane phospholipase A (OMPLA), ferric hydroxamate uptake component A (FhuA), Curli production transport component CsgG, or a Neisseria autotransporter lipoprotein (NalP).

36. A method, comprising:
(a) providing a system comprising (i) a fluidic chamber; (ii) a membrane that separates the fluidic chamber into a first side and a second side; and (iii) a nanopore disposed in the membrane,
wherein the nanopore comprises (1) at least a portion of an alpha helical pore forming protein or peptide, or (2) at least a portion of a beta barrel pore forming protein or peptide, wherein the at least the portion of the beta barrel pore forming protein or peptide is not an alpha-hemolysin or is not an aerolysin, wherein the at least the portion of the alpha helical pore forming protein or peptide or the at least the portion of the beta barrel pore forming protein or peptide comprises a modification of one or more lumen facing amino acids into one or more natural or non-natural aromatic amino acids; and
(b) bringing the nanopore in contact with an analyte.

37. The method of claim 36, further comprising translocating the analyte through the nanopore.

38. The method of claim 37, further comprising measuring a signal generated by translocating the analyte through the nanopore.

39. The method of claim 36, wherein the nanopore is brought in contact with the analyte using an electro-osmotic force.

40. The method of claim 36, wherein the nanopore comprises the at least the portion of the alpha helical pore forming protein or peptide.

41. The method of claim 36, wherein the at least the portion of the alpha helical pore forming protein or peptide comprises an actinoporin.

42. The method of claim 36, wherein the nanopore comprises the at least the portion of the beta barrel pore forming protein or peptide.

43. The method of claim 36, wherein the at least the portion of the beta barrel pore forming protein or peptide comprises a cytolysin, leukocidin, bacterial outer membrane porin, or de novo designed pore peptide.

44. The method of claim 36, wherein the analyte comprises a non-nucleic acid analyte.

45. The method of claim 36, wherein the analyte comprises a peptide, an oligopeptide, or a protein.

46. The method of claim 36, wherein the at least the portion of the beta barrel pore forming protein or peptide comprises a Cytolysin K, lysenin, Anthrax toxin, Mycobacterium smegmatis porin A (MspA), Mycobacterium smegmatis porin B (MspB), Mycobacterium smegmatis porin C (MspC), Mycobacterium smegmatis porin D (MspD), outer membrane porin F (OmpF), outer membrane porin G (OmpG), outer membrane phospholipase A (OMPLA), ferric hydroxamate uptake component A (FhuA), Curli production transport component CsgG, or a Neisseria autotransporter lipoprotein (NalP).


SEQUENCE LISTING (101 sequences)

SEQ ID NO: 1 - protein, 179 aa, Actinia fragacea
SADVAGAVIDGAGLGFDVLKTVLEALGNVKRKIAVGIDNESGKTWTAMNTYFRSGTSDIVLPHKVAHGKALLYNGQKNRGPVATGVVGVIAYSMSDGNTLAVLFSVPYDYNWYSNWWNVRVYKGQKRADQRMYEELYYHRSPFRGDNGWHSRGLGYGLKSRGFMNSSGHAILEIHVTKA

SEQ ID NO: 2 - protein, 20 aa, Actinia fragacea
SAEVAGAVIEGAKLTFNVLQ

SEQ ID NO: 3 - protein, 167 aa, Actinia fragacea
SLTFDVLQTVLKALGDVSRKIAVGIDNEPGMTWTAMNTYFRSGTSDVILPHTVPHSKALLYDGQKNRGPVTTGVVGVIAYAMSDGNTLAVLFSIPFDYNLYSNWWNVKVYKGHRRADQAMYEELYYDFSPFRGDNGWHTKSIGYGLKGRGFMNSSGKAILQIHVNKV

SEQ ID NO: 4 - protein, 20 aa, Actinia fragacea
SVAVAGAVIKGAALTFNILQ

SEQ ID NO: 5 - protein, 168 aa, Actinia fragacea
AGLGFDVLKTVLEALGNVKRKIAVGIDNESGRTWTAMNTYFRSGTSDIVLPHKVAHGKALLYNGQKNRGPVATGVVGVIAYSMSDGNTLAVLFSVPYDYNWYSNWWNVRVYKGQKRANQRMYEELYYHRSPFRGDNGWHSRSLGYGLKSRGFMNSSGHAILEIHVTKA

SEQ ID NO: 6 - protein, 179 aa, Actinia equina
SADVAGAVIDGASLSFDILKTVLEALGNVKRKIAVGVDNESGKTWTALNTYFRSGTSDIVLPHKVPHGKALLYNGQKDRGPVATGAVGVLAYLMSDGNTLAVLFSVPYDYNWYSNWWNVRIYKGKRRADQRMYEELYYNLSPFRGDNGWHTRNLGYGLKSRGFMNSSGHAILEIHVSKA

SEQ ID NO: 7 - protein, 179 aa, Actinia equina
SVAVAGAIIKGAALTFNVLQTVLKALGDISRKIAVGVDNESGKTWTALNTYFRSGTSDIVLPHKVPHGKALLYNGQKDRGPVATGAVGVLAYAMSDGNTLAVLFSVPYDYNWYSNWWNVRIFKGRRRADQRMYEQLYYYLSPFRGDNGWHERHLGYGLKSRGFMNSGGQAILEIHVTKA

SEQ ID NO: 8 - protein, 179 aa, Actinia equina
SVAVAGAVIEGATLTFNVLQTVLKALGDISRKIAVGIDNESGMTWTAMNTYFRSGTSDVILPHTVPHGKALLYNGQKDRGPVATGVVGVLAYAMSDGNTLAVLFSIPFDYNLYSNWWNVKVYKGHRRADQRMYEELYYNLSPFRGDNGWHNRDLGYGLKGRGFMNSSGQSILEIHVTKA

SEQ ID NO: 9 - protein, 179 aa, Urticina felina
  source 1..179: note = Urticina crassicornis
SVAIAGAVIEGAKLTFGILEKILTVLGDINRKIAIGVDNESGREWTAQNAYFFSGTSDVVLPASVPNTKAFLYNAQKDRGPVATGVVGVLAYSLSNGNTLGILFSVPYDYNLYSNWWNIKLYKGIKRADRDMYNDLYYYAHPHKGDNGWHENSLGFGLKSKGFMTSSGQTILQIRVSRA

SEQ ID NO: 10 - protein, 173 aa, Oulactis orientalis
GAIIAGAALGFNVHQTVLKALGQVSRKIAIGVDNESGGTWTALNAYFRSGTTDVILPEFVPNQKALLYSGQKDTGPVATGAVGVLAYYMSDGNTLGVMFSVPFDYNLYSNWWDVKVYRGRRRADQAMYEGLLYGIPYGGDNGWHARKLGYGLKGRGFMKSSAQSILEIHVTKA

SEQ ID NO: 11 - protein, 165 aa, Oulactis orientalis
ATFRVLAKVLAELGKVSRKIAVGVDNESGGSWTALNAYFRSGTTDVILPDLVPNQKALLYRGGKDTGPVATGVVGVLAYAMSDGNTLAILFSVPYDYNLYSNWWNVKVYSGKRRADQGMSEDLSYGNPYGGDNGWHARKLAYGLKERGFMKSSAQSILEIHATKA

SEQ ID NO: 12 - protein, 177 aa, Stichodactyla gigantea
ASAVAGTIIEGASLTFQILDKVLTELGNVSRKIAIGIDNESGGSWTAMNAYFRSGTTDVILPEFVPNNKALLYSGRKDTGPVTTGAVGALAYYMSDGNTLAVMFSVPFDYNLYSNWWDVRVYSGKRRADQKMYEDLYNGSPFKGDNGWHQKNLGYGLRMKGIMTSAGEAKLQIKISR

SEQ ID NO: 13 - protein, 177 aa, Heteractis magnifica
SAALAGTIIEGASLGFQILDKVLGELGKVSRKIAVGVDNESGGSWTALNAYFRSGTTDVILPEFVPNQKALLYSGRKDTGPVATGAVAAFAYYMSNGHTLGVMFSVPFDYNFYSNWWDVKVYSGKRRADQGMYEDMYYGNPYRGDNGWHQKNLGYGLRMKGIMTSAGEAILQIRISR

SEQ ID NO: 14 - protein, 179 aa, Anthopleura asiatica
SLAVAGAVIEGGNLVMSVLDRILEAIGDVNRKIAIGVENQSGKSWTAMNTYFRSGTSDVVLPHSVPSGKALLYDGQKTRGPVATGVVGVFAYAMSDGNTLAVMFSIPYDYNLYSNWWNVKTYSGMKRADQSMYEDLYYHASPFKGDNGWHSRNLGYGLKCRGFMNSSGAAKLEIHVSRA

SEQ ID NO: 15 - protein, 201 aa, Cribrinopsis japonica
LPMKEDISNEERPTSVNEKPVKKSVAVAGAVIQGAALAFQVLDKILTSLGGIGRKIAIGVDNESGMKWAARNVYFYSGTSDTVLPYSVPHSKAFLYGARKTRGSVRGAVGVLAYSMSDGNTLGILFSVPYDYNWYSNWWNIKVYRGYKRANKWMYHDLYYYARPHKGNNEWHEKSLGYGLKSKGFMTSSGQTKLEIRVSRA

SEQ ID NO: 16 - protein, 202 aa, Cribrinopsis japonica
LPMKEDISNDERPISVNEEPVKKNAAVAGAVIQGATLTFQVLDRILTVLGDISRKIAIGVDNESGRKWTAKNAYFFSGTSDVVLPYSVPNGKAFLYDGKKTRGPVATGAVGVLAYSMSDGNTLGILFSVPYDYNWYENWWNIKVYSGSKRANKWMYENLYYNASPHKGDNGWHEKSLGYGLKSRGYMASSGQTKLEIRVTRA

SEQ ID NO: 17 - protein, 176 aa, Stichodactyla helianthus
SELAGTIIDGASLTFEVLDKVLGELGKVSRKIAVGIDNESGGTWTALNAYFRSGTTDVILPEVVPNTKALLYSGRKSSGPVATGAVAAFAYYMSNGNTLGVMFSVPFDYNWYSNWWDVKIYPGKRRADQGMYEDMYYGNPYRGDNGWYQKNLGYGLRMKGIMTSAGEAKMQIKISR

SEQ ID NO: 18 - protein, 175 aa, Stichodactyla helianthus [sequence omitted]

SEQ ID NO: 19 - protein, 175 aa, Heteractis crispa
ALAGAIIAGASLTFQILDKVLAELGQVSRKIAIGIDNESGGSWTAMNAYFRSGTTDVILPEFVPNQKALLYSGRKNRGPDTTGAVGALAYYMSNGNTLGVMFSVPFDYNLYSNWWDVKVYSGKRRADQAMYEDLYYSNPYRGDNGWHQKNLGYGLKMKGIMTSAGEAIMEIRISR

SEQ ID NO: 20 - protein, 177 aa, Heteractis crispa
SAALAGTITLGASLGFQILDKVLGELGKVSRKIAVGVDNESGGSWTALNAYFRSGTTDVILPEFVPNQKALLYSGRKDTGPVATGAVAAFAYYMSNGHTLGVMFSVPFDYNLYSNWWDVKIYSGKRRADQAMYEDMYYGNPYRGDNGWHQKNLGYGLKMKGIMTSAVEAILEIRISR

SEQ ID NO: 21 - protein, 178 aa, unidentified
  source 1..178: note = Sagartia rosea
KISGGTVIAAGRLTLDLLKTLLGTLGSISRKIAIGVDNETGGLITGNNVYFRSGTSDDILPHRVETGEALLYTARKTKGPVATGAVGVFTYYLSDGNTLAVLFSVPFDYNFYSNWWNVKIYSGKRNADYDMYHELYYDANPFEGDDTWEYRYLGYGMRMEGYMNSPGEAILKITVMPD

SEQ ID NO: 22 - protein, 179 aa, Actineria villosa
SAAVAGAVIAGGELALKILTKILDEIGKIDRKIAIGVDNESGLKWTALNTYYKSGASDVTLPYEVENSKALLYTARKSKGPVARGAVGVLAYKMSSGNTLAVMFSVPFDYNLYSNWWNVKIYDGEKKADEKMYNELYNNNNPIKPSTWEKRDLGKDGLKLRGFMTSNGDAKLVIHIEKS

SEQ ID NO: 23 - protein, 179 aa, Phyllodiscus semoni
SAAVAGAVIAGGELALKILTKILDEIGKIDRKIAIGVDNESGLKWTALNTYYKSGASDVTLPYEVENSKALLYTARKSKGPVARGAVGVLAYKMSSGNTLAVMFSVPFDYNLYTNWWNVKIYDGEKKADEKMYNELYNNNNPIKPSIWEKRDLGQDGLKLRGFMTSNGDAKLVIHIEKS

SEQ ID NO: 24 - protein, 193 aa, Anthopleura nigrescens
LPLEEKEDEKDEKRSLEVAGAVMEGANLGMSVLQTILQAIGDVSRKIAVGVDNESGRSWTAQNAYFRSGTSDVILPHTVPSGKALLYDGQKNRGPVATGVVGVITYTMGDGNTLAVMFSVPYDYNWYSNWWNVKIYHGKVRASQKMYEDLYYYRSPFKGDNGWHERNLGYGLKSKGFMNSSGAALLQIKVMKA

SEQ ID NO: 25 - protein, 493 aa, Aeromonas hydrophila
MQKIKLTGLSLIISGLLMAQAQAAEPVYPDQLRLFSLGQGVCGDKYRPVNREEAQSVKSNIVGMMGQWQISGLANGWVIMGPGYNGEIKPGTASNTWCYPTNPVTGEIPTLSALDIPDGDEVDVQWRLVHDSANFIKPTSYLAHYLGYAWVGGNHSQYVGEDMDVTRDGDGWVIRGNNDGGCDGYRCGDKTAIKVSNFAYNLDPDSFKHGDVTQSDRQLVKTVVGWAVNDSDTPQSGYDVTLRYDTATNWSKTNTYGLSEKVTTKNKFKWPLVGETELSIEIAANQSWASQNGGSTTTSLSQSVRPTVPARSKIPVKIELYKADISYPYEFKADVSYDLTLSGFLRWGGNAWYTHPDNRPNWNHTFVIGPYKDKASSIRYQWDKRYIPGEVKWWDWNWTIQQNGLSTMQNNLARVLRPVRAGITGDFSAESQFAGNIEIGAPVPLAADSKVRRARSVDGAGQGLRLEIPLDAQELSGLGFNNVSLSVTPAANQ

SEQ ID NO: 26 - protein, 337 aa, Bacillus cereus
MKRSKTYLKCLALSAVFASSALALSTPAAYAQTTSQVVTDIGQNAKTHTSYNTFNNDQTDNMTMSLKVTFIDDPSADKQIAVINTTGSFLKANPTISSAPIDNYPIPGASATLRYPSQYDIAFNLQDNSARFFNVAPTNAVEETTVTSSVSYQLGGSVKASATPNGLSAEAGATGQVTWSDSVSYKQTSYKTNLIDQTNKNVKWNVFFNGYNNQNWGIYTRDSYHSLYGNQLFMYSRTYLYESDAKGNLIPMDQLPALTNSGFSPGMIAVVISEKNTDQSNLQVAYTKHADDYQLRPGFTFGTANWVGNNVKDVDQKTFNKSFTLDWKNKKLVEKNR

SEQ ID NO: 27 - protein, 317 aa, Staphylococcus aureus
MKTRIVSSVTTTLLLGSILMNPVAGAADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEM

SEQ ID NO: 28 - protein, 311 aa, Staphylococcus aureus
MNKLVKSSVATSMALLLLSGTANAEGKITPVSVKKVDDKVTLYKTTATADSDKFKISQILTFNFIKDKSYDKDTLVLKATGNINSGFVKPNPNDYDFSKLYWGAKYNVSISSQSNDSVNVVDYAPKNQNEEFQVQNTLGYTFGGDISISNGLSGGLNGNTAFSETINYKQESYRTTLSRNTNYKNVGWGVEAHKIMNNGWGPYGRDSFHPTYGNELFLAGRQSSAYAGQNFIAQHQMPLLSRSNFNPEFLSVLSHRQDGAKKSKITVTYQREMDLYQIRWNGFYWAGANYKNFKTRTFKSTYEIDWENHKV

SEQ ID NO: 29 - protein, 309 aa, Staphylococcus aureus
MNKLVKSSVATSMALLLLSGTANAEGKITPVSVKKVDDKVTLYKTTATADSDKFKISQILTFNFIKDKSYDKDTLVLKATGNINSGFVKPNPNDYDFSKLYWGAKYNVSISSQSNDSVNAVDYAPKNQNEEFQVQNTLGYTFGGDISISNGLSGGLNGNTAFSETINYKQESYRTLSRNTNYKNVGWGVEAHKIMNGWGPYGRDSFHPTYGNELFLAGRQSSAYAGQNFIAQHQMPLLSRSNFNPEFLSVLSHRQDRAKKSKITVTYQREMDLYQIRWNGFYWAGANYKNFKTRTFKSTYEIDWENHKV

SEQ ID NO: 30 - protein, 308 aa, Staphylococcus aureus
IEKLGKSSVASSIALLLLSNTVDAAQNITPKREKKVDDKITLYKTTATSDNDKLNIFQILTFNFIKDKSYDKDTLVLKAAGNINSGYKNSNPKDYNYSQFYWGGKYNVSVSSESNDAVNVVDYAPKNQNEEFQVQQTLGYSYGGDINISNGLSGGLNGSKSFSETINYKQESYRTTIDRKTNHKSIGWGVEAHKIMNNGWGPYGRDSYDPTYGNELFLGGDKSSSNAGQNFLPTHQIPLLARGNFNPEFISVLSHKLFDTKKSKIKVTYQREMDRYTNQWNRSHWVGNNYKNQNTVTFTSTYEVDWQN

SEQ ID NO: 31 - protein, 262 aa, Staphylococcus aureus
IKNKILTATLAVGLIAPLANPFIEISKAENKIEDIGQGAEIIKRTQDITSKRLAITQNIQFDFVKDKKYNKDALVVKMQGFISSRTTYSDLKKYPYIKRMIWPFQYNISLKTKDSNVDLINYLPKNKIDSADVSQKLGYNIGGNFQSAPSIGGSGSFNYSKTISYNQKNYVTEVESQNSKGVKWGVKANSFVTPNGQVSAYDQYLFAQDPTGPAARDYFVPDNQLPPLIQSGFNPSFITTLSHERGKGDKSEFEITYGRNMD

SEQ ID NO: 32 - protein, 275 aa, Staphylococcus aureus
DIGKGSDIEIIKRTEDKTSNKWGVTQNIQFDFVKDTKYNKDALILKMQGFISSRTTYYNYKKTNHVKAMRWPFQYNIGLKTNDKYVSLINYLPKNKIESTNVSQTLGYNIGGNFQSAPSLGGNGSFNYSKSISYTQQNYVSEVEQQNSKSVLWGVKANSFATESGQKSAFDSDLFVGYKPHSKDPRDYFVPDSELPPLVQSGFNPSFIATVSHEKGSSDTSEFEITYGRNMDVTHAIKRSTHYGNSYLDGHRVHNAFVNRNYTVKYEVNWKTHEI

SEQ ID NO: 33 - protein, 275 aa, Staphylococcus aureus
DIGKGSDIEIIKRTEDKTSNKWGVTQNIQFDFVKDKKYNKDALILKMQGFISSRTTYYNYKKTNHVKAMRWPFQYNIGLKTNDKYVSLINYLPKNKIESTNVSQILGYNIGGNFQSAPSLGGNGSFNYSKSISYTQQNYVSEVEQQNSKSVLWGVKANSFATESGQKSAFDSDLFVGYKPHSKDPRDYFVPDSELPPLVQSGFNPSFIATVSHEKGSSDTSEFEITYGRNMDVTHAIKRSTHYGNSYLDGHRVHNAFVNRNYTVKYEVNWKTHEI

SEQ ID NO: 34 - protein, 212 aa, Staphylococcus aureus
VTQNVQFDFVKDKKYNKDALIVKMQGFINSRTSFSDVKGSGYELTKRMIWPFQYNIGLTTKDPNVSLINYLPKNKIETTDVGQTLGYNIGGNFQSAPSIGGNGSFNYSKTISYTQKSYVSEVDKQNSKSVKWGVKANEFVTPDGKKSAHDRYLFVQSPNGPTGSAREYFAPDNQLPPLVQSGFNPSFITTLSHEKGSSDTSEFEISYGRNLD

SEQ ID NO: 35 - protein, 296 aa, Eisenia fetida
MSAKAAEGYEQIEVDVVAVWKEGYVYENRGSTSVDQKITITKGMKNVNSETRTVTATHSIGSTISTGDAFEIGSVEVSYSHSHEESQVSMTETEVYESKVIEHTITIPPTSKFTRWQLNADVGGADIEYMYLIDEVTPIGGTQSIPQVITSRAKIIVGRQIILGKTEIRIKHAERKEYMTVVSRKSWPAATLGHSKLFKFVLYEDWGGFRIKTLNTMYSGYEYAYSSDQGGIYFDQGTDNPKQRWAINKSLPLRHGDVVTFMNKYFTRSGLCYDDGPATNVYCLDKREDKWILEVV

SEQ ID NO: 36 - protein, 5 aa, synthetic construct
  REGION 1..5: note = Leucine-enkephalin.
YGGFL

SEQ ID NO: 37 - protein, 5 aa, synthetic construct
  REGION 1..5: note = Angiotensin II (4-8)
YIHPF

SEQ ID NO: 38 - protein, 7 aa, synthetic construct
  REGION 1..7: note = Kemptide
LRRASLG

SEQ ID NO: 39 - protein, 7 aa, synthetic construct
  REGION 1..7: note = phosphorylated kemptide
  SITE 5: note = MOD_RES - PHOSPHORYLATION
LRRASLG

SEQ ID NO: 40 - protein, 9 aa, synthetic construct
  REGION 1..9: note = unmodified peptide
ANVTLNTAG

SEQ ID NO: 41 - protein, 9 aa, synthetic construct
  REGION 1..9: note = peptide with one glycan
  SITE 4: note = MOD_RES - T is modified with glycan
ANVTLNTAG

SEQ ID NO: 42 - protein, 9 aa, synthetic construct
  REGION 1..9: note = peptide with two glycans
  SITE 4: note = MOD_RES - T is modified with glycan
  SITE 8: note = MOD_RES - T is modified with glycan
ANVTLNTTG

SEQ ID NO: 43 - protein, 9 aa, synthetic construct
  REGION 1..9: note = peptide fragment
SGRNAAVVK

SEQ ID NO: 44 - protein, 9 aa, synthetic construct
  REGION 1..9: note = rhamnosylated peptide
  SITE 3: note = MOD_RES - R is rhamnosylated
SGRNAAVVK

SEQ ID NO: 45 - protein, 7 aa, synthetic construct
  REGION 1..7: note = modified enkephalin
  SITE 2: note = MISC_FEATURE - is D-Asp
  SITE 6: note = MISC_FEATURE - is D-Asp
YDAGFDL

SEQ ID NO: 46 - DNA, 19 bp, synthetic construct
  misc_feature 1..19: note = T7 reverse primer
  source 1..19: mol_type = other DNA
gctagttattgctcagcgg

SEQ ID NO: 47 - protein, 190 aa, synthetic construct
  REGION 1..190: note = His6-tagged wild type Fragaceatoxin C.
MASADVAGAVIDGAGLGFDVLKTVLEALGNVKRKIAVGIDNESGKTWTAMNTYFRSGTSDIVLPHKVAHGKALLYNGQKNRGPVATGVVGVIAYSMSDGNTLAVLFSVPYDYNWYSNWWNVRVYKGQKRADQRMYEELYYHRSPFRGDNGWHSRGLGYGLKSRGFMNSSGHAILEIHVTKAGSAHHHHHH

SEQ ID NO: 48 - protein, 14 aa, synthetic construct
  REGION 1..14: note = Angiotensinogen peptide
DRVYIHPFHLVIHN

SEQ ID NO: 49 - protein, 10 aa, synthetic construct
  REGION 1..10: note = Angiotensin 1 peptide
DRVYIHPFHL

SEQ ID NO: 50 - protein, 7 aa, synthetic construct
  REGION 1..7: note = Angiotensin 3 peptide
RVYIHPF

SEQ ID NO: 51 - protein, 6 aa, synthetic construct
  REGION 1..6: note = Angiotensin 4 peptide
VYIHPF

SEQ ID NO: 52 - protein, 27 aa, synthetic construct
  REGION 1..27: note = Common sequence alignment of some known actinoporins
  VARIANT 2: note = MISC_FEATURE - X can be A, V, L, I, or S
  VARIANT 24: note = MISC_FEATURE - X can be E, T, K, G, A or Q
  VARIANT 25: note = MISC_FEATURE - X can be A, V, E, T or S
SXAVAGAVIEGASLTFQVLDKVLXXLG

SEQ ID NO: 53 - protein, 27 aa, Actinia fragacea
SADVAGAVIDGAGLGFDVLKTVLEALG

SEQ ID NO: 54 - protein, 27 aa, Urticina felina
  source 1..27: note = Urticina crassicornis
SVAIAGAVIEGAKLTFGILEKILTVLG

SEQ ID NO: 55 - protein, 27 aa, Actinia equina
SADVAGAVIDGASLSFDILKTVLEALG

SEQ ID NO: 56 - protein, 27 aa, Actinia equina
SVAVAGAIIKGAALTFNVLQTVLKALG

SEQ ID NO: 57 - protein, 27 aa, Actinia equina
SVAVAGAVIEGATLTFNVLQTVLKALG

SEQ ID NO: 58 - protein, 27 aa, Anthopleura asiatica
SLAVAGAVIEGGNLVMSVLDRILEAIG

SEQ ID NO: 59 - protein, 27 aa, Heteractis magnifica
SAALAGTIIEGASLGFQILDKVLGELG

SEQ ID NO: 60 - protein, 25 aa, Heteractis crispa
ALAGAIIAGASLTFQILDKVLAELG

SEQ ID NO: 61 - protein, 27 aa, Heteractis crispa
SAALAGTITLGASLGFQILDKVLGELG

SEQ ID NO: 62 - protein, 26 aa, unidentified
  source 1..26: note = Sagartia rosea
KISGGTVIAAGRLTLDLLKTLLGTLG

SEQ ID NO: 63 - protein, 26 aa, Stichodactyla helianthus
SELAGTIIDGASLTFEVLDKVLGELG

SEQ ID NO: 64 - protein, 25 aa, Stichodactyla helianthus
ALAGTIIAGASLTFQVLDKVLEELG

SEQ ID NO: 65 - protein, 27 aa, Cribrinopsis japonica
SVAVAGAVIQGAALAFQVLDKILTSLG

SEQ ID NO: 66 - protein, 27 aa, Cribrinopsis japonica
NAAVAGAVIQGATLTFQVLDRILTVLG

SEQ ID NO: 67 - protein, 27 aa, Anthopleura nigrescens
SLEVAGAVMEGANLGMSVLQTILQAIG

SEQ ID NO: 68 - protein, 27 aa, unidentified
  source 1..27: note = Stychodactyla gigantea
ASAVAGTIIEGASLTFQILDKVLTELG

SEQ ID NO: 69 - protein, 23 aa, Actinia fragacea
SADVAGAVIDGAGLGFDVLKTVL

SEQ ID NO: 70 - protein, 23 aa, Anthopleura asiatica
SLAVAGAVIEGGNLVMSVLDRIL

SEQ ID NO: 71 - protein, 46 aa, Cribrinopsis japonica
LPMKEDISNDERPISVNEEPVKKNAAVAGAVIQGATLTFQVLDRIL

SEQ ID NO: 72 - protein, 46 aa, Cribrinopsis japonica
LPMKEDISNEERPTSVNEKPVKKSVAVAGAVIQGAALAFQVLDKIL

SEQ ID NO: 73 - protein, 23 aa, Actineria villosa
SAAVAGAVIAGGELALKILTKIL

SEQ ID NO: 74 - protein, 23 aa, Phyllodiscus semoni
SAAVAGAVIAGGELALKILTKIL

SEQ ID NO: 75 - protein, 23 aa, Actinia equina
SADVAGAVIDGASLSFDILKTVL

SEQ ID NO: 76 - protein, 23 aa, Actinia equina
SVAVAGAIIKGAALTFNVLQTVL

SEQ ID NO: 77 - protein, 23 aa, Actinia equina
SVAVAGAVIEGATLTFNVLQTVL

SEQ ID NO: 78 - protein, 23 aa, unidentified
  source 1..23: note = Stychodactyla gigantea
ASAVAGTIIEGASLTFQILDKVL

SEQ ID NO: 79 - protein, 23 aa, Heteractis magnifica
SAALAGTIIEGASLGFQILDKVL

SEQ ID NO: 80 - protein, 5 aa, Anthopleura nigrescens
LPLEE

SEQ ID NO: 81 - protein, 32 aa, Anthopleura nigrescens
KEDEKDEKRSLEVAGAVMEGANLGMSVLQTIL

SEQ ID NO: 82 - protein, 21 aa, Heteractis crispa
ALAGAIIAGASLTFQILDKVL

SEQ ID NO: 83 - protein, 23 aa, Heteractis crispa
SAALAGTITLGASLGFQILDKVL

SEQ ID NO: 84 - protein, 22 aa, unidentified
  source 1..22: note = Sagartia rosea
KISGGTVIAAGRLTLDLLKTLL

SEQ ID NO: 85 - protein, 21 aa, Stichodactyla helianthus
ALAGTIIAGASLTFQVLDKVL

SEQ ID NO: 86 - protein, 22 aa, Stichodactyla helianthus
SELAGTIIDGASLTFEVLDKVL

SEQ ID NO: 87 - protein, 23 aa, Urticina felina
  source 1..23: note = Urticina crassicornis
SVAIAGAVIEGAKLTFGILEKIL

SEQ ID NO: 88 - protein, 6 aa, synthetic construct
  REGION 1..6: note = peptide fragment
RGNAEK

SEQ ID NO: 89 - protein, 5 aa, synthetic construct
  REGION 1..5: note = peptide fragment
FDQGK

SEQ ID NO: 90 - protein, 5 aa, synthetic construct
  REGION 1..5: note = peptide fragment
HIYVR

SEQ ID NO: 91 - protein, 5 aa, synthetic construct
  REGION 1..5: note = peptide
TPGSR

SEQ ID NO: 92 - protein, 7 aa, synthetic construct
  REGION 1..7: note = peptide
HGLDNYR

SEQ ID NO: 93 - protein, 8 aa, synthetic construct
  REGION 1..8: note = peptide
CELAAAMK

SEQ ID NO: 94 - protein, 7 aa, synthetic construct
  REGION 1..7: note = peptide
WWCNDGR

SEQ ID NO: 95 - protein, 9 aa, synthetic construct
  REGION 1..9: note = peptide
GTDVQAWIR

SEQ ID NO: 96 - protein, 12 aa, synthetic construct
  REGION 1..12: note = peptide
GYSLGNWVCAAK

SEQ ID NO: 97 - protein, 12 aa, synthetic construct
  REGION 1..12: note = peptide
FESNFNTQATNR

SEQ ID NO: 98 - protein, 13 aa, synthetic construct
  REGION 1..13: note = peptide fragment
NLLTGAGTETVFK

SEQ ID NO: 99 - protein, 5 aa, synthetic construct
  REGION 1..5: note = [Ala2]-Leu Enkephalin
YAGFL

SEQ ID NO: 100 - protein, 5 aa, synthetic construct
  REGION 1..5: note = DADLE ([D-Ala2, D-Leu5]-Enkephalin)
  SITE 2: note = MISC_FEATURE - is D-Ala
  SITE 5: note = MISC_FEATURE - is D-Leu
YAGFL

SEQ ID NO: 101 - protein, 6 aa, synthetic construct
HHHHHH